(19) United States
(12) Patent Application Publication
(10) Pub. No.: US 2025/0144383 A1
(43) Pub. Date: May 8, 2025

(54) INNER EAR DEVICE WITH ACCESS AND PASSIVE COMPONENTS

(71) Applicant: COCHLEAR LIMITED, Macquarie University, NSW (AU)

(72) Inventors: Wolfram Frederik DUECK, Macquarie University (AU); Daniel SMYTH, Macquarie University (AU)

(21) Appl. No.: 18/684,331

(22) PCT Filed: Aug. 20, 2022
(86) PCT No.: PCT/IB2022/057825

§ 371 Date: Feb. 16, 2024

Related U.S. Application Data
(60) Provisional application No. 63/235,322, filed on Aug. 20, 2021.

(51) Int. Cl. A61M 31/00

(52) U.S. Cl. CPC A61M 31/002; A61M 2202/0007; A61M 2205/04; A61M 2205/3331; A61M 2210/0668

(57) ABSTRACT

A device including a tissue interface portion configured for securement to tissue of and/or proximate an inner ear of a human and provide a passage from outside the inner ear to inside the inner ear and a component releasably attached to the tissue interface portion and/or a portion of the device supported by the tissue interface portion, wherein the device is configured to enable the component to be removed from the tissue interface portion when the tissue interface portion is removably permanently fixed to the barrier establishing the inner ear of a human, and the component at least partially seals the passage and provides one or more passive features.

### CROSS-REFERENCE TO RELATED APPLICATIONS

This application claims priority to U.S. Provisional Application No. 63/235,322, entitled INNER EAR DEVICE WITH ACCESS AND PASSIVE COMPONENTS, filed on Aug. 20, 2021, naming Wolfram Frederik DUECK of Hannover, Germany as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a device, comprising: a tissue interface portion configured for securement to tissue of and/or proximate an inner ear of a human and provide a passage from outside the inner ear to inside the inner ear; and a component releasably attached to the tissue interface portion and/or a portion of the device supported by the tissue interface portion, wherein the device is configured to enable the component to be removed from the tissue interface portion when the tissue interface portion is removably permanently fixed to the barrier establishing the inner ear of a human, and the component at least partially seals the passage and provides one or more passive features.

In an exemplary embodiment, there is a system, comprising: a tissue interface apparatus through which a passage extends, wherein the tissue interface apparatus is configured to permanently fix to an opening in a barrier between a middle ear and an inner ear of a human such that the passage extends from the middle ear to the inner ear; and a first functional component at least partially located in the passage, wherein at least one of:
the first functional component is a passive first functional component detachably attached directly or indirectly to the tissue interface apparatus and removal of the first component provides physical access from the middle ear into the inner ear through the passage, wherein the first functional component at least partially seals the passage in addition to providing a passive feature;
the tissue interface apparatus includes a second passage that is unsealably sealed, the second passage providing physical access from the middle ear into the inner ear through the second passage bypassing the first functional component; or
the first functional component has a passage that is unsealably sealed, the passage of the first functional component providing physical access from a middle ear facing side to an inner ear facing side when unsealed with the first functional component in the passage of the tissue interface apparatus.

In an exemplary embodiment, there is an inner ear port apparatus, comprising: an elongate tapered metallic body through which a passage extends from a proximal end of the body to a distal end of the body, wherein an outer surface of the body along a longitudinal direction of the body includes threads or ribs configured to grip bone establishing a barrier between a middle ear and an inner ear of a human to permanently fix the body to an opening in the barrier between the middle ear and the inner ear, the inner ear port apparatus that is configured to enable resealable physical access from the middle ear into the inner ear through the passage, and the inner ear port apparatus includes at least one of a component with passive functionality that at least partially seals the passageway or a component with passive functionally and a separate seal apparatus.

In an exemplary embodiment, there is a device, comprising: a tissue interface portion configured for securement to tissue of and/or proximate an inner ear of a human and provide a passage from outside the inner ear to inside the inner ear; a removable seal apparatus configured to unsealably seal the passage; and a component releasably attached to the tissue interface portion and/or a portion of the device supported by the tissue interface portion, the component providing one or more passive features, wherein the device is configured to enable the component to be removed from the tissue interface portion when the tissue interface portion is removably permanently fixed to the barrier establishing the inner ear of a human.

In an exemplary embodiment, there is a system, comprising: a tissue interface apparatus through which a passage extends, wherein the tissue interface apparatus is configured to permanently fix to an opening in a barrier between a middle ear and an inner ear of a human such that the passage extends from the middle ear to the inner ear; and a first functional component at least partially located in the passage, wherein at least one of:
the first functional component is a passive first functional component detachably attached directly or indirectly to the tissue interface apparatus and removal of the first component provides physical access from the middle ear into the inner ear through the passage, wherein the system further includes a removable seal apparatus configured to unsealably seal the passage, the removable seal apparatus being separate from the first functional component;
the tissue interface apparatus includes a second passage that is unsealably sealed, the second passage providing physical access from the middle ear into the inner ear through the second passage bypassing the first functional component; or
the first functional component has a passage that is unsealably sealed, the passage of the first functional component providing physical access from a middle ear facing side to an inner ear facing side when unsealed with the first functional component in the passage of the tissue interface apparatus.

In exemplary embodiment, there is method, comprising: obtaining access, at a location within a middle ear of a human, to an implanted port that provides access to an inner ear from the middle ear of a human, wherein the port openably closes a passageway between the inner ear and the middle ear, wherein the port has been implanted in the human for at least one month; and at least one of:
after at least 10 days of the port being implanted in the human, removably attaching to the port a component, and then detaching that component after at least 5 days after attaching the component to the port;
removing a first component that is attached to the port and replacing the first component with a second component that executes a function in a passive manner or that is a plug, wherein the first component executes at least one of an active function or a passive function; or
piercing a septum that openably closes the passageway with a termination of a syringe, and transferring a substance, foreign to the human, from outside the inner ear into the inner ear through the port, via the termination.

### BRIEF DESCRIPTION OF THE DRAWINGS

Embodiments are described below with reference to the attached drawings, in which:

FIG. 1 is perspective view of a human ear;

FIG. 2 is a perspective view of an exemplary cochlear stimulator implanted in accordance with an exemplary embodiment;

FIGS. 3 and 4 and 4A are schematics depicting exemplary implantable components for background purposes;

FIG. 5 is a schematic depicting an exemplary therapeutic substance delivery system for background purposes;

FIG. 6 is a schematic depicting exemplary background working ends of an embodiment that combines the embodiments of FIGS. 3 to 5.

FIGS. 7-14A and 16-33 are schematics depicting exemplary embodiments according to the invention;

FIG. 15 is a schematic depicting insertion of an electrode array through a port in an exemplary embodiment;

FIG. 34 presents exemplary flowcharts for exemplary methods; and

FIGS. 35-37 are schematics depicting exemplary embodiments according to the invention.

### DETAILED DESCRIPTION

Merely for ease of description, the techniques presented herein are sometimes described herein with reference to an illustrative medical device, namely a cochlear stimulator, and in other instances, a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. Some embodiments include the utilization of the teachings herein to treat an inner ear of a recipient that has and/or utilizes one or more of these devices. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein can be implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, cochlear stimulator, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

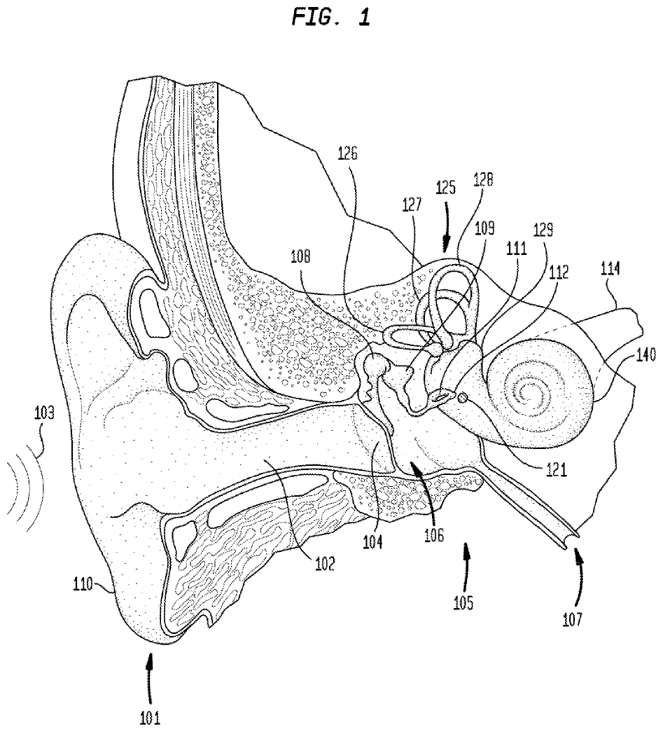

FIG. 1 is a perspective view of a human skull showing the anatomy of the human ear. As shown in FIG. 1, the human ear comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

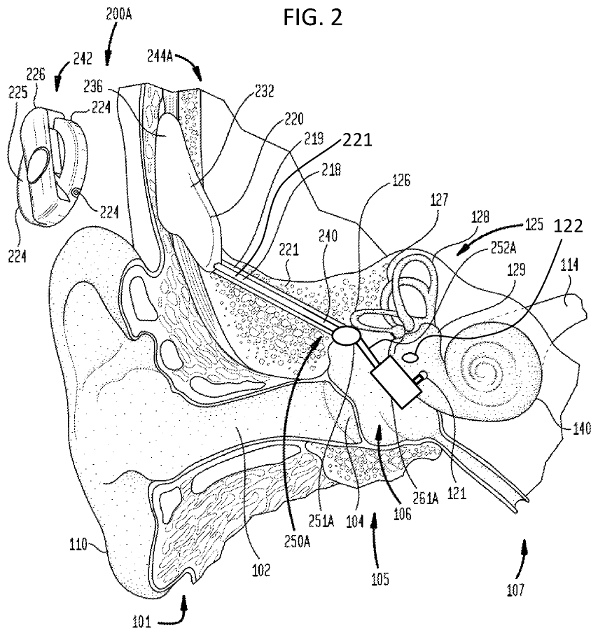

FIG. 2 is a perspective view of an exemplary cochlear stimulator 200A in accordance with some exemplary embodiments. Cochlear stimulator 200A comprises an external component 242 that is directly or indirectly attached to the body of the recipient, and an internal component 244A that is temporarily or permanently implanted in the recipient. External component 242 typically comprises two or more sound input elements, such as microphones 224 for detecting sound, a sound processing unit 226, a power source (not shown), and an external transmitter unit 225. External transmitter unit 225 comprises an external coil (not shown). Sound processing unit 226 processes the output of microphones 224 and generates encoded data signals which are provided to external transmitter unit 225. For ease of illustration, sound processing unit 226 is shown detached from the recipient.

Internal component 244A comprises an internal receiver unit 232, a stimulator unit 220, and a stimulation arrangement 250A in electrical communication with stimulator unit 220 via cable 218 extending thorough artificial passageway 219 in mastoid bone 221. Internal receiver unit 232 and stimulator unit 220 are hermetically sealed within a biocompatible housing, and are sometimes collectively referred to as a stimulator/receiver unit.

Internal receiver unit 232 comprises an internal coil (not shown), and optionally, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated platinum or gold wire. The electrical insulation of the internal coil is provided by a flexible silicone molding (not shown). In use, implantable receiver unit 232 is positioned in a recess of the temporal bone adjacent auricle 110.

In the illustrative embodiment of FIG. 2, ossicles 106 have been explanted, thus revealing oval window 122.

Stimulation arrangement 250A comprises both the distal and proximal portions of cable 218 (221 and 240), an actuator assembly 261A, an actuator mount member 251A, an actuator position arm 252A that extends from actuator mount member 251A and supports or at least holds actuator assembly 261A in place relative to the outside of the cochlea 140. In an exemplary embodiment, actuator mount member 251A is osseointegrated to mastoid bone 221, or more particularly, to the exit of artificial passageway 219 formed in mastoid bone 221.

In this embodiment, stimulation arrangement 250A is implanted and/or configured such that a portion of the actuator assembly interfaces with the round window 121, as can be seen, while it is noted that in an alternate embodiment, a portion of the actuator assembly interfaces with the oval window 122 (and both windows in some alternate embodiments).

As noted above, a sound signal is received by microphone(s) 224, processed by sound processing unit 226, and transmitted as encoded data signals to internal receiver 232. Based on these received signals, stimulator unit 220 generates drive signals which cause actuation of actuator assembly 261A.

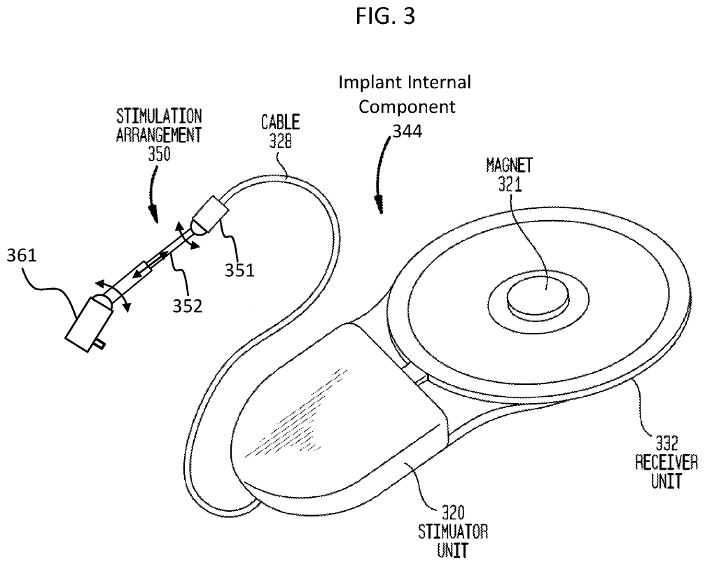

FIG. 3 is a perspective view of an exemplary internal component 344 of an implant which generally represents internal component 244A described above. Internal component 344 comprises an internal receiver unit 332, a stimulator unit 320, and a stimulation arrangement 350. As shown, receiver unit 332 comprises an internal coil (not shown), and a magnet 321 fixed relative to the internal coil. In some embodiments, internal receiver unit 332 and stimulator unit 320 are hermetically sealed within a biocompatible housing. This housing has been omitted from FIG. 3 for ease of illustration.

Stimulator unit 320 is connected to stimulation arrangement 350 via a cable 328, corresponding to cable 218 of FIG. 2. Stimulation arrangement 350 comprises an actuator assembly 361, corresponding to actuator 261A of FIG. 2, an actuator assembly mount member 351, corresponding to actuator assembly mount member 251A of FIG. 2, and an actuator assembly positioning arm 352, corresponding to the actuator assembly positioning arm 352 of FIG. 2. In an exemplary embodiment, actuator assembly mount member 351 is configured to be located in the artificial passageway 219 or adjacent thereto and fixed to the mastoid bone of the recipient. As indicated by the curved arrows of FIG. 3, the actuator assembly mount member 351 and the actuator assembly 361 are configured to enable articulation of the actuator assembly positioning arm 352 relative to those components. Further, as indicated by the straight arrow of FIG. 3, the actuation assembly positioning arm 352 is configured to telescope to provide longitudinal adjustment between the actuator assembly 361 and the actuator assembly mount member 251.

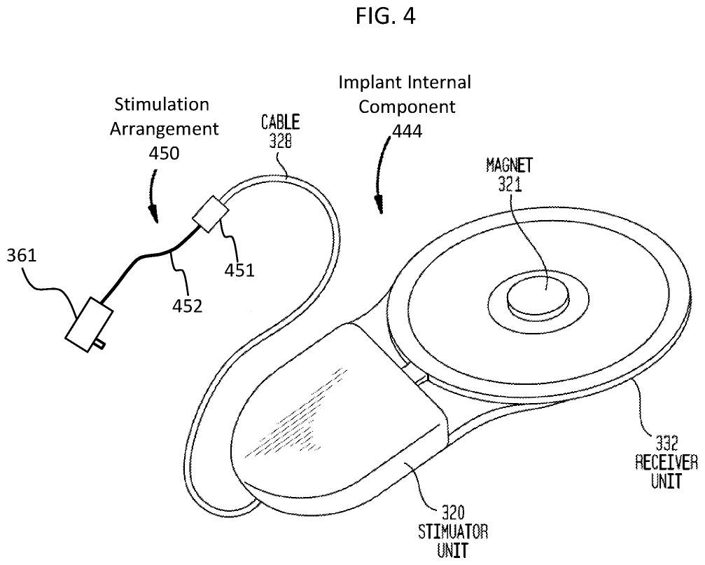

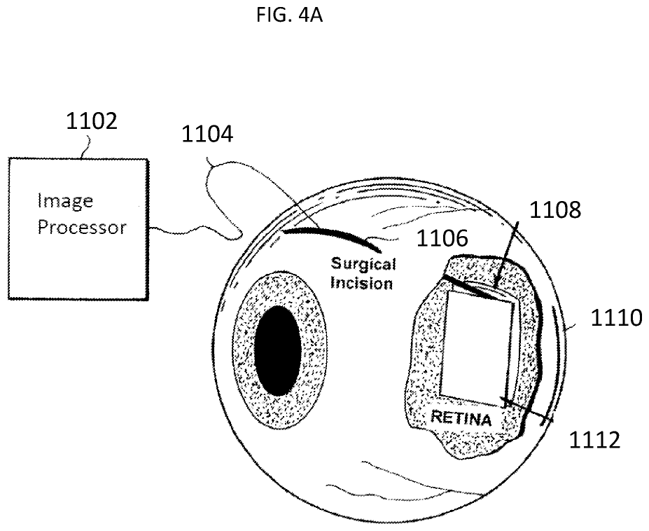

FIG. 4 is a perspective view of an exemplary internal component 444 of an implant which generally represents internal component 244A described above. Internal component 444 comprises like components corresponding to those of internal component 344.

As with internal component 344, internal component 444 is such that stimulator unit 320 is connected to stimulation arrangement 450 via a cable 328, corresponding to cable 218 of FIG. 2. However, element 451 is a coupling that instead of coupling to the articulation device detailed above in the embodiment of FIG. 3, couplies to cable 452 which is coupled to actuator assembly 361. This embodiment provides a less complicated arrangement which can have utilitarian value where the surgeon or the like is going to hand connect actuator assembly 361 directly to the exterior of the cochlea and where actuator assembly 361 will remain in place relative to the cochlea for a given period of time. The cable 452 is flexible so as to permit relative ease of movement of the actuator assembly 361 during the implantation process. The coupling 451 enables the stimulation arrangement 350 to be replaced without removing the stimulator unit 320 and/or enables the stimulator unit 320 to be removed and replaced without removing the stimulation arrangement 450.

FIG. 4A presents an exemplary embodiment of a neural prosthesis in general, and a retinal prosthesis and an environment of use thereof, in particular. In some embodiments of a retinal prosthesis, a retinal prosthesis sensor-stimulator 1108 is positioned proximate the retina 1110. In an exemplary embodiment, photons entering the eye are absorbed by a microelectronic array of the sensor-stimulator 1108 that is hybridized to a glass piece 1112 containing, for example, an embedded array of microwires. The glass can have a curved surface that conforms to the inner radius of the retina. The sensor-stimulator 108 can include a microelectronic imaging device that can be made of thin silicone containing integrated circuitry that convert the incident photons to an electronic charge.

An image processor 1102 is in signal communication with the sensor-stimulator 1108 via cable 1104 which extends through surgical incision 1106 through the eye wall (although in other embodiments, the image processor 1102 is in wireless communication with the sensor-stimulator 1108). In an exemplary embodiment, the image processor 1102 is analogous to the sound processor/signal processors of the auditory prostheses detailed herein, and in this regard, any disclosure of the latter herein corresponds to a disclosure of the former in an alternate embodiment. The image processor 1102 processes the input into the sensor-stimulator 108, and provides control signals back to the sensor-stimulator 1108 so the device can provide processed and output to the optic nerve. That said, in an alternate embodiment, the processing is executed by a component proximate to or integrated with the sensor-stimulator 1108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 1108 captures light/images, which sensor-stimulator is implanted in the recipient. In an exemplary embodiment, there is a transcutaneous communication coil that is held against a skin of a recipient via magnetic attraction to communication with an implanted component, which implanted component provides the stimulation to evoke a sight precept. In an embodiment, the teachings herein regarding magnetic attraction are utilized in such.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

Some exemplary embodiments of the teachings detailed herein enable drug delivery to the cochlea or otherwise the delivery of a utilitarian substance to the cochlea.

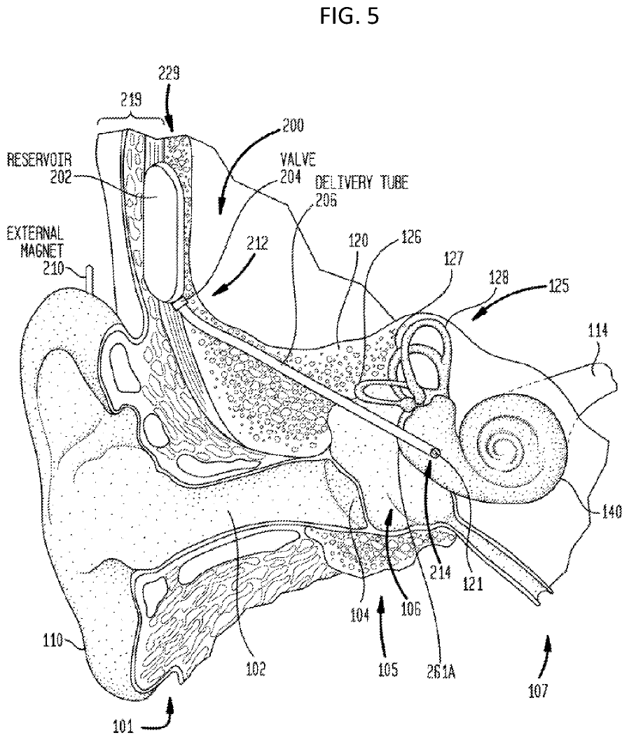

FIG. 5 depicts an exemplary drug delivery device, the details of which will be provided below. It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

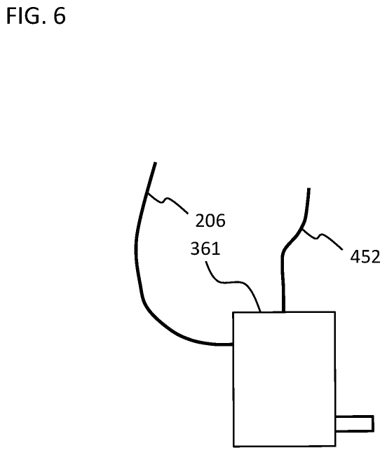

FIG. 5 illustrates an implantable delivery system 200 having an actuation mechanism, which can be modified as will be detailed below in some embodiments. However, it is noted that the delivery system 200 can also or instead have an active actuation system, again which can be modified as will be detailed below. The delivery system 200 is sometimes referred to herein as an inner ear delivery system because it is configured to deliver treatment substances to the recipient's inner ear (e.g., the target location is the interior of the recipient's cochlea 140). It is also noted that in some implementations of a modified arrangement of FIG. 5, as will be described below, the actuation mechanism enables movement of therapeutic substance to another device that in turn has an active actuation mechanism (e.g., element 361 of FIG. 6A, additional details of which are described below), where the latter is used to actually transport the therapeutic substance into a cochlea (the former is used to get the substances to the latter).

Delivery system 200 of FIG. 5 comprises a reservoir 202, a valve 204, and a delivery tube 206, in addition to some additional components, as will be described below. For ease of illustration, the delivery system 200 is shown separate from any implantable auditory prostheses. Additionally, the delivery system 200 can include, or operate with, an external magnet 210, which is separate from or part of the implantable auditory prostheses, for purposes of, e.g., controlling operation of valve 204.

The delivery tube 206 includes a proximal end 212 and a distal end 214. The proximal end 212 of the delivery tube 206 is fluidically coupled to the reservoir 202 via the valve 204.

FIG. 5, as shown, utilizes an actuation mechanism to produce a pumping action to transfer a treatment substance from the reservoir 202 to the delivery device 208 at the distal end 214 of the delivery tube 206, but again, some embodiments are modified versions of FIG. 5 that utilize active actuation.

In some implementations of FIG. 5, external force is applied on the tissue 219 adjacent to the reservoir 202 to create the external force. As will be described below, in some embodiments, an external vibratory device of a passive transcutaneous bone conduction device that vibrates to evoke a hearing percept is pressed onto the soft tissue 219 under which the reservoir 202 is located. The movement (e.g., oscillation/vibration) of the actuator causes deformations the reservoir 202 to create the pumping action that propels the treatment substance out of the reservoir.

As noted, the treatment substance (sometimes herein referred to as therapeutic substance) is released from the reservoir 202 through the valve 204. The valve 204 may be a check valve (one-way valve) that allows the treatment substance to pass therethrough in one direction only.

Once the treatment substance is released through valve 204, the treatment substance flows through the delivery tube 206 to the cochlea, either directly, or indirectly via the actuator assembly 361/461. In embodiments utilizing the actuator assembly, the actuator assembly corresponds to a transfer mechanism to transfer the treatment substance from the delivery tube 206 into the cochlea 140 via the round window 121 (or oval window, or another orifice such as that established by a cochleostomy into the cochlea).

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

As noted herein, the therapeutic delivery system of FIG. 5 can be combined with a partially or fully implanted device configured to evoke a hearing percept. By way of example only and not by way of limitation, the therapeutic delivery system of FIG. 5 can be combined with the hearing prosthesis of FIG. 3 and FIG. 4. Briefly, in an exemplary embodiment, the actuator assembly 361 can be configured so as to receive or otherwise connect to the distal end of tube 206 of the therapeutic delivery system. In an exemplary embodiment of such as depicted in FIG. 6A, where the embodiment of FIG. 4 is presented by way of example, it is to be understood that the embodiment of FIG. 6 is also applicable to the embodiment of FIG. 3.

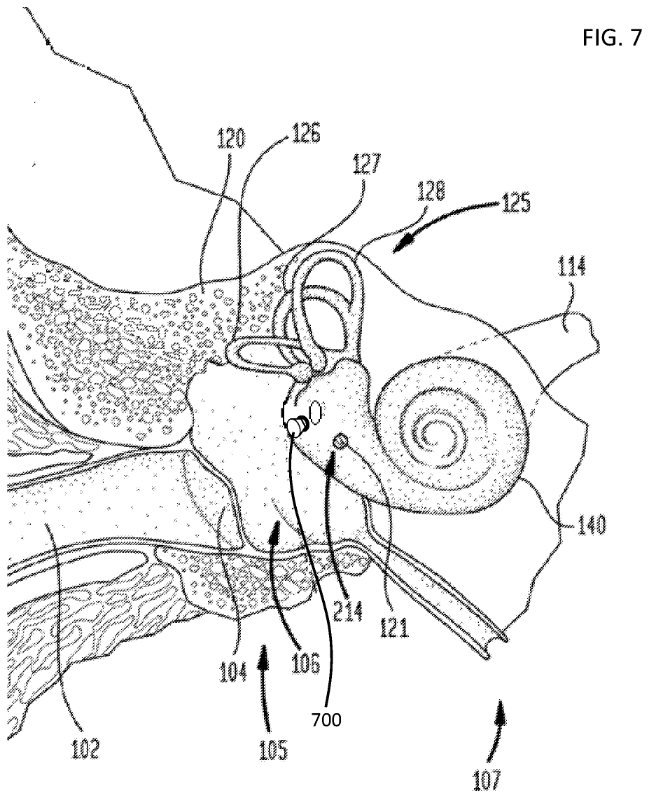

FIG. 7 presents an exemplary embodiment that is different than that disclosed in FIGS. 5-6. In this regard, the invention of this patent application corresponds to the embodiments of FIG. 7 and the figures thereafter. Any means-plus-function claims relating to the implant as a whole correspond to the structure of FIG. 7 and/or the figures thereafter. It is noted that some exemplary embodiments of the invention utilize the structure and/or function of the teachings detailed above. And embodiments of the implants according to the invention can include one or more of the above noted structures and/or functions and/or can include methods that include one or more of the above noted method actions. However, with respect to the implant, the invention does not include the implants detailed above. This is thus related art that some aspects of the invention can utilize.

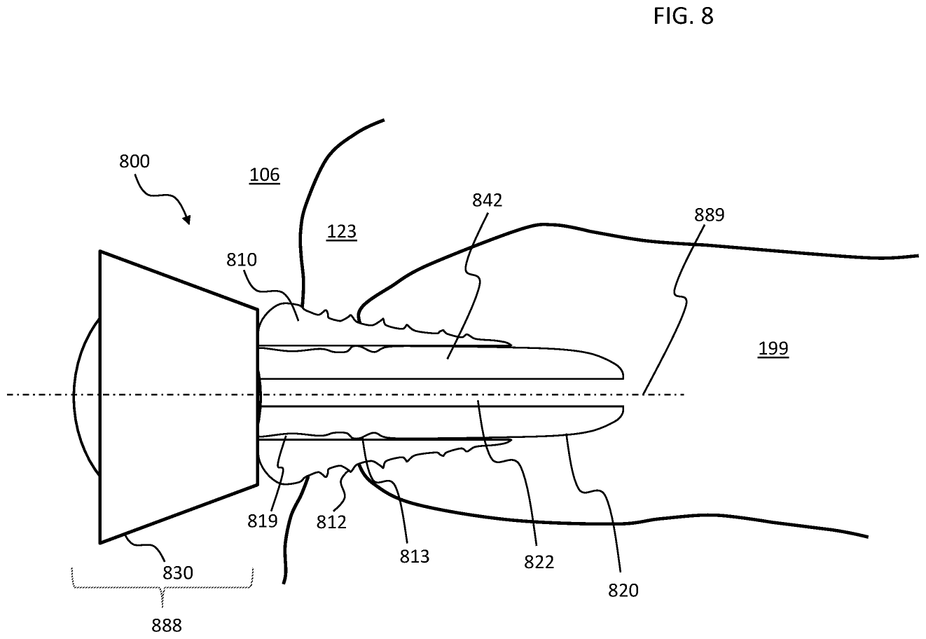

It is also briefly noted that in FIG. 7, the ossicles have been removed (from the figure) in the interest of clarity. Some embodiments can be utilized with an intact ossicles, while other embodiments are utilized in a human where the ossicles of the respective middle ear cavity has been removed. To be clear, embodiments according to the teachings detailed herein are directed towards preserving hearing or otherwise treating hearing loss, and thus in some embodiments, the ossicles are present and functioning. But it is noted that the absence of the ossicles does not rule out embodiments associated with preserving hearing and/or treating hearing loss-hearing could be established via a middle ear implant and/or a bone conduction implant and/or a cochlear implant electrode array, etc. For example, embodiments of the teachings detailed herein can be utilized to preserve or otherwise prevent cilia degradation, where the ossicles have completely deteriorated to the point of not being useful from a medical standpoint-they might be there, but they do not function in a medically meaningful way for example. It is briefly noted by way of background that, in general, absent the teachings associated with FIG. 7 and FIG. 8 (more on this below) and variations thereof, access to the inner ear has the potential to cause damage to hearing and/or balance. Moreover, again absent the teachings associated with FIGS. 7 and 8, repeated access to try different therapies, or for repeated application of drugs, can often create added risk. For example, drug delivered to the middle ear is poorly transferred to the inner ear (thus meaning that the efficacy can be relatively low, for example). In general, providing a drug treatment to the inner ear by placing drug in the middle ear is challenging. Some access the cochlea using cochlear implant techniques and sheath introducers. Hearing drug companies often attempt to deliver to the middle ear using gels delivered to the inner ear with single shot approaches. Indeed, the standard of care for drug delivery to the ear today is middle ear injection in solution. Often, hearing drug companies try to improve delivery to the inner ear by using gels in the middle ear or by using single shot direct cochlear injection (direct in that the termination contacts directly the tissue establishing a barrier between the middle ear and the inner ear).

The teachings herein can address the above issues, at least in some embodiments. In this regard, in an exemplary embodiment, as seen in FIGS. 7 and 8, there is an inner ear port device 700. FIG. 7 depicts the visible portions of an exemplary inner ear port device 700 visible from the middle ear 106 cavity. The port device is configured to enable resealable physical access from the middle ear cavity 106 into the inner ear 199 (see FIG. 8) through a passage through the port device 700. In an exemplary embodiment, the port device 700 is configured to enable the resealable physical access at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 times or more, or any value or range of therebetween in 1 increment (e.g., 47, 66, 33 to 176, etc.). In an exemplary embodiment, the port device is configured to meet one or more of the aforementioned quantities within 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 weeks and/or months from the date of implantation of the inner ear port device into the human. Corollary to this is that there are methods of accessing the port device any one or more of the aforementioned quantities within any one or more of the aforementioned temporal periods.

At least some embodiments of the teachings herein enable inner ear access while maintaining cochlear function. At least some embodiments enable smart therapeutic substance delivery to the inner ear. At least some embodiments enable smart therapeutic substance delivery to the eye system, such as the eye shown in FIG. 4A (in an exemplary embodiment, the port devices can be implanted in an eye bone/skull bone proximate the eye and/or the nerves extending from the eye(s), and thus some embodiments can be an eye prosthesis. Some embodiments can enable implantation of such devices in the eyeball by pushing through to the vitreous humor. Accordingly, any disclosure of interfacing with an inner ear and/or providing therapeutic substance thereto and/or sensing phenomena thereof, etc., corresponds to an alternate disclosure of interfacing with body tissue associated with and/or proximate the eye(s) and/or optical nervous system, for purposes of textual economy.

Some exemplary structure of an exemplary embodiment of the port device 700 will now be described.

FIG. 8 depicts a side view partial cross-sectional view of an exemplary embodiment of an inner ear port device 800 which can correspond to the inner ear port device 700 noted above, which extends from the middle ear cavity 106, through the bone structure 123, that divides the middle ear cavity 106 from the interior of the cochlea 199, and thus extends therethrough. The port device can extend through the promontory. The port device can extend through the barrier between the middle ear and the inner. The port device can extend through the wall of the first turn of the cochlea. The port device can extend through the bone between the round and oval window. In this embodiment, the port device 800 includes a portion that is located in or otherwise is accessible from the middle ear cavity 106. Also as seen, the port device 800 includes a portion that is located in or otherwise is in fluid communication with the cavity 199 of the cochlea, which can be one or more of the three ducts of the cochlea. In an exemplary embodiment, therapeutic substances can be transferred from a location within the cavity 106 into the cavity 199 through the port 800.

It is briefly noted that by “transferred from a location,” this includes the scenario where the therapeutic substance travels through that area from a location that originates outside of the middle ear cavity 106. For example, a syringe including a substance can be located in the outer ear, and the termination can extend through the tympanic membrane, across cavity 106, and into the port device 800. Upon operating the syringe to transfer the therapeutic substance therein from the outer ear to the inner ear, the therapeutic substance passes through the middle ear 106, and thus is transferred from a location in the middle ear. This is as distinguished from a therapeutic substance that has as its origin location within the middle ear cavity 106, which could be the case with respect to a reservoir that is part of the port device, which reservoir is entirely located in the middle ear 106 (this would also include the species of the substance being transferred from the location within the middle ear 106—this would not include the species of the substance having an origination at the time of being attached or otherwise introduced to the body at a location outside the middle ear).

In at least some exemplary embodiments, the port device 800 is attached to the wall of the cochlea 123 at a location away from the round window and/or from the oval window. In this regard, the passage through the wall the cochlea 123 can be established via a cochleostomy through the bony structure of the cochlea 123. That said, in at least some exemplary embodiments, the port device 800 can extend through the wall of the cochlea at the location of the round window or oval window (two can be used at both locations in some embodiments), more accurately, or potentially, the former location of the round window or oval window. Thus, in some embodiments, the device is located in a cochleostomy away from a natural round window location of a human.

FIG. 8 depicts a body 810. A passage 819 extends through that body. While in at least some exemplary embodiments, the passage 819 can include only a seal apparatus, such as by way of example, a septum, such as a self-healing septum, some additional details of which will be described below, in this exemplary embodiment, the passage 819 has a second component, here, module 820, located therein, which module in turn has a passage 822. Body 830 is screwably attached to module 820, which body forms a head of an assembly that includes module 820 (the assembly can be considered itself a module-thus, there is a first module, body 810, and a second module that is the assembly of head 888 and element 820 (or, just element 820 can be considered the second module)). In an exemplary embodiment, pulling on the head 888 pulls out the element 820 from the passage through the body 810. That said, in some embodiments, element 830/head 888 is separate from module 820. In the embodiments depicted in FIG. 8, both passages extend from inside the cavity 199 to outside the cavity 106. This as compared to, for example, other embodiments where one or both of the passages extend only to the cavity 199 and/or to the cavity 106 (the passage would stop at the interface/extrapolated interface, of the cavitie(s)). This also as compared to, for example, other embodiments, where one or both of the passages do not even extend to the respective cavities, where, for example, the “end”/“beginning” of the respective passages stop short/begin after the interface/extrapolated interface, of the cavitie(s). It is also noted that the aforementioned features associated with the passages can also be applicable to the overall body configuration.

In the embodiment of FIG. 8, the body 810 is configured to fix to an opening in the barrier between the middle ear in the inner ear of the human (e.g., the cochleostomy). In an exemplary embodiment, the body 810 is configured to permanently fix to an opening in the barrier.

Briefly, as seen in FIG. 8, the body 810 includes one or more protrusions 812 that can extend circumferentially about the body and/or can be located at discrete portions on the outer surface of the body 810 (e.g., they could be barbs, or spikes), and thus a combination can be utilized in some embodiments. In this embodiment, the protrusions 812 can be ribs that can have sharp edges, which will grip the bone 123 or other tissue with which the body 810 interfaces. In an exemplary embodiment, the protrusions 812 can instead be a single screw thread (and thus there would be one protrusion) and/or a plurality of screw threads, thus enabling the body 810 to be screwed into the passage. In an exemplary embodiment, the body is rotationally symmetric about the longitudinal axis 889 in its entirety (in some embodiments) and/or aside from the protrusion(s), and can be made of titanium or a titanium alloy or some other biocompatible material having sufficient longevity with respect to the intended environment (e.g., implanted as shown for 5 or 10 or 20 years, etc.). The body can be a turned and/or a casted metal body and/or 3D printed, for example, or extrusion molded, or cast molded, all by way of example. The body 810 can be turned from a thick-walled and/or a thin-walled tube. For example, a tube of titanium can be obtained, and then cut to the desired length, and then the tapered feature can be cut from the tube by turning the tube on a lathe. In an exemplary embodiment, the body 810 is a monolithic component (e.g., cut from a single tube). In an exemplary embodiment, the body can be a unified structure made from two or more components that are joined together (e.g., a portion of the body establishing a left component can be screwed into a component that establishes the right portion of the body, a “bottom” half (e.g., the portion below the axis 889) of the body can be snapped coupled to the top half of the body.

In an exemplary embodiment, the screw thread(s) of the inner ear port device can be self-tapping screw threads, more accurately, a tissue interface portion, such as body 810, of the inner ear port device can be configured as a self-tapping screw arrangement. Thus, embodiments include establishing a passageway through the bone between the middle ear cavity and the inner ear cavity without drilling. That is, by way of example only and not by way of limitation, at the location where an inner ear device as detailed herein and/or variations thereof are positioned, the first time that the barrier between the inner ear and the middle ear at that location is breached is by the inner ear port device.

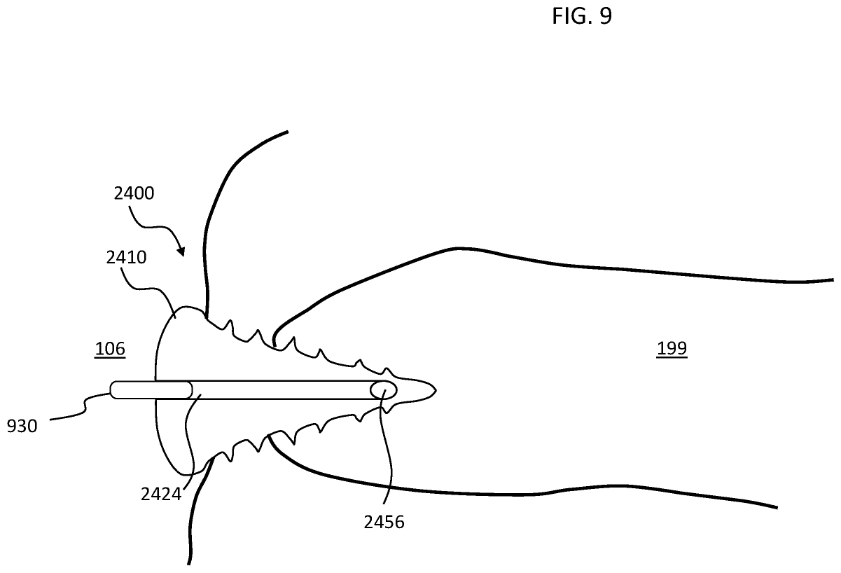

FIG. 9 presents an exemplary inner ear port device 2410 that has the self-tapping and/or self-threading and/or-self screwing features according to some embodiments (any reference to self-tapping corresponds to a disclosure of an alternate embodiment of self-threading and/or self-screwing and vis-a-versa, unless otherwise specified). Here, there is a passageway 2424 that extends from the proximal end of the body 2410 (the body 2410 can have any one or more of the features of the body 1810 detailed above) to the outlet orifice 2456, which is located on a side of the body 2410 (the conduit established by passage 2424 and the orifice 2456 can be created by, for example, drilling only partially into the embryonic body 2410 along the longitudinal axis thereof, and then separately drilling from the side of the embryonic body 2410 at an angle of 90° to the longitudinal axes down to the end of the passage 2424) so as to provide for a “solid” the tip of the body for the purposes of self-tapping. Also shown is a plug 930, such as a temporary plug, that operates according to some of the exemplary plugs detailed herein. The plug 930 can be placed partially in the passageway 2424 as shown so as to seal the passageway, temporarily. A portion of the plug 930 can stick out of the passageway 2424 as shown, enabling the plug to be removed and/or installed by gripping the end of the plug with a tweezers and/or a forceps or tools designed for use in middle ear surgery, for example, and moving the plug accordingly.

In an alternate exemplary embodiment of a self-tapping port device (which can be a self-threading port device in an alternate embodiment), the passageway 2424 can be offset from the longitudinal axis of the body 2410. In this regard, in an exemplary embodiment, if passageway 2424 is centered at one third of the distance from the longitudinal axis and the outermost portion of the body 2410, then the opening of the passage facing the distal end would be away from the end/tip, thus enabling the self-tapping feature to be present (the passage 2424 can be completely drilled from the proximal face of the embryonic body 2410 all the way through to the other side, where, once the drill bit fully passes through the embryonic body, the tip and sufficient portions there about still remain completely intact. Thus, in some exemplary embodiments, the outlet hole can be eccentric, and, in some embodiments, the passage can be eccentric.

In an exemplary embodiment, the self-tapping feature and/or the self-drilling feature can provide utilitarian value. Depending on the embodiment, there can be no need to drill first or otherwise first establish a passageway through the bone separating the middle ear cavity with the inner ear cavity, because the body 2410 or any other body that has these features establishes the passageway itself, and thus the cochlea remains fluidically sealed at all times during insertion and after the port is installed, at least until the passageway is unsealed for example. Accordingly, in an exemplary embodiment, there is the action of establishing a passageway from the middle ear to the inner ear while the cochlea remains fluidically sealed during the action of establishing the passageway and for at least 0.25, 0.5, 1, 2, 3, 4, 5, 6, 7, 8, 9, 10, 15, 20, 25, 30, 40, 50 minutes, or 1, 2, 3, 4, 5, 6, 7, 8, 9, 10, 20, 40, or 100 hours, or any value or range of is therebetween in 0.05 minute increments. In an exemplary embodiment, for at least 1, 2, 3, 4, 5, 6, 7, 8, 9, 10, 11, 12, 13, 14, or 15 months after the action of establishing the passageway utilizing the inner ear port device, the cochlea remain sealed.

Alternatively, the body could be a polymer or some form of biocompatible synthetic material. In an exemplary embodiment, the body could be made of PEEK. In an exemplary embodiment, the bodies can be casted or otherwise formed of these materials, or alternatively, cut from a larger body of these materials. In some embodiments, a coating such as Hydroxyapatite and/or titanium can be placed over the base material of the body. Titanium coatings or some other biocompatible metallic coating can be used.

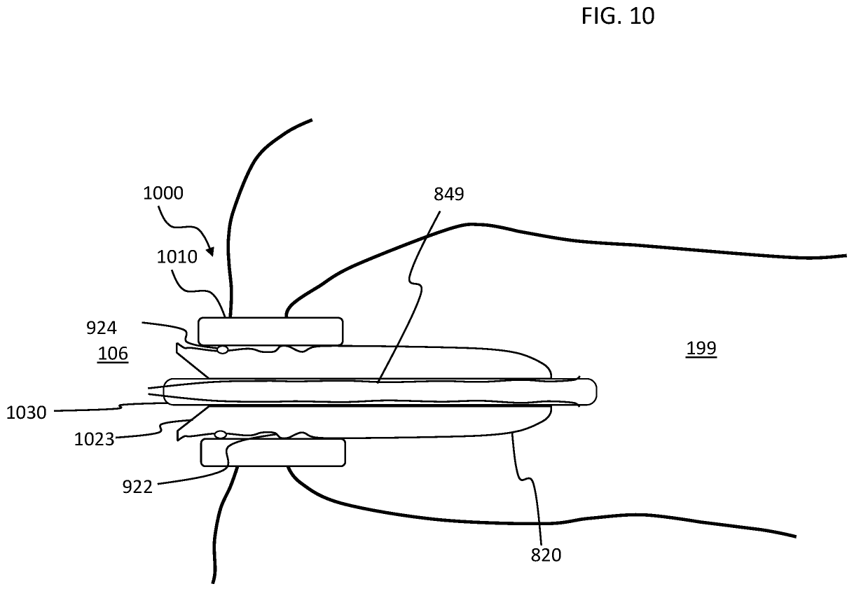

While the embodiment depicted in FIG. 8 shows a tapered body, other embodiments could have an outer surface that maintains the same distance from the longitudinal axis for at least a portion of its length, such as the portion that extends through the passage. This is seen in FIG. 10, which shows a cross-section of a body 1010 of a port 1000, which cross-section lies on it is parallel to the longitudinal axis of the body. This embodiment does not show the protrusions 812, but in other embodiments, the protrusions can be located on the outer surface. Note also and that this exemplary embodiment, an interference fit or a press fit or a strain/yield (plastic and/or elastic) fit can be utilized. In an exemplary embodiment, the outer diameter of the body 1010 can be slightly larger than the interior diameter of the passage. Utilizing a sufficiently deformable material, the body 1010 can establish the aforementioned fits and thus be secured in the passage. That said, in an alternate embodiment, the underlying deform ability of the bone can be relied upon to establish the interference fit and/or yield fit. It is noted that such fits can also be applicable to the tapered body of FIG. 8. It is also noted that such fits can be utilized in combination with the protrusions.

In an exemplary embodiment, the body could be shrunken first, such as by way of example only and not by way of limitation by chilling, and then inserted into the passage. Upon warming to body temperature, the body would then expand, and establish one of the aforementioned fits. And while the embodiments shown have the outer surface of the body that is located inside the cavity being no larger than the portions that are in the passage, in other embodiments, the portion of the body that is located inside the cavity 199 can be larger, and, in some embodiments, by utilizing the aforementioned chilling method, that larger portion could also be fit through the passage, and then upon expansion, a positive retention regime could be obtained (e.g., like a nail head. It is noted that the outer diameter of the body can also be larger than that which is in the passage with respect to portions of the body located in the cavity 106.

Any device or arrangement that can enable the functionality of the body can be utilized in at least some exemplary embodiments providing such is enabled by the art unless otherwise noted.

In at least some exemplary embodiments, the body 810 is configured to enable a seal between the body and the bone 123. In some exemplary embodiments, such as where, for example, there is a modicum of flexibility with respect to the structure of the body 810, the body itself can be utilized to establish the seal. In an exemplary embodiment, the protrusions 812 can be configured so as to dig into the bone and establish a seal as a result of the fact that the protrusions essentially force themselves into the bone. In an exemplary embodiment, such as where osseointegration is experienced, the osseointegration and/or fibrosis can establish a seal. Still further, as seen in FIG. 9, separate seals can be utilized, such as O-ring seal 816. Further, the sealing compound can be utilized in an exemplary embodiment, the services of the passageway that is drilled or otherwise bored through the bone 123 can be coated with a substance that will establish a seal between the body 810 and the service of the passageway through the bone 123. Any device, system, and/or method that can enable the establishment of a seal between the cavity 106 and the cavity 199 vis-à-vis the outer surface of the body and the bone 123 can be utilized in at least some exemplary embodiments providing that the art enables such and such presents a biocompatible way of doing so, unless otherwise noted.

And in the interest of clarity, with respect to the phrase “seal,” it is noted that that means that a barrier is established that presents a medically efficacious barrier with respect to the movement of substances from the cavity 106 to the cavity 199 and vice versa, which substances are normally expected to be found in the middle ear and/or in the inner ear, such as by way of example only and not by way of limitation, perilymph with respect to the latter, and possibly, bacteria with respect to the former (where the seal provides utilitarian value with respect to preventing bacteria that is located or otherwise present in the middle ear from reaching the inner ear all by way of example).

As noted above, some exemplary embodiments are directed towards a body that is configured to permanently fix to an opening in the barrier between the middle ear in the inner ear of a human. By “permanently fix,” it is meant that the body can remain in the human for at least a year, if not longer, such as, for example, any one or more of the aforementioned longer temporal periods noted above, at the location that it is implanted at the time of implantation. This as distinguished from, for example, a temporary component/a temporary port, that might be utilized for only a few hours or a few days or a few weeks after implantation, and/or a device that could dissolve or degrade owing to the fluids of the body, or otherwise has a reliability engineering design such that functionality is likely to degrade to an un-functional state in a statistically significant number of designs.

And there is a middle ground, for example, where the device is configured to be “healed out” of the cochlea wall, for example. For example, the port could be designed to be pushed out by reforming bone after a few months or more (say after 3, 4, 5, 6, 7, 8, 9, 10, 11, 12 or more months). And note that this could be a scenario where the port is configured or otherwise of the design to be permanently implanted vis-à-vis the temporal periods detailed herein with respect to the mere ability to stay implanted without causing a deleterious effect on the human, but where the application thereof results in healing out of the port. But the design of the port can be configured for such result as well. That is, the port can be configured to have a shape for example that will result in the healing out of the port based on normal bone regrowth. To be clear, the two designs are not mutually exclusive to each other. The material and/or the design of the port can be configured to satisfy the longevity requirements, even though the device is not utilized for such long temporal periods.

Any reference to the features associated with longevity/permanence of the body are also applicable to one or more or all of the other components/portions of the port device unless otherwise noted, providing that the art enables such.

The fixation can be established by any of the regimes detailed above or below in an exemplary embodiment, the body is made of a material that osseointegrates to the bone, and thus in some embodiments the body is osseointegrated to the bone to achieve the aforementioned permanent fixation.

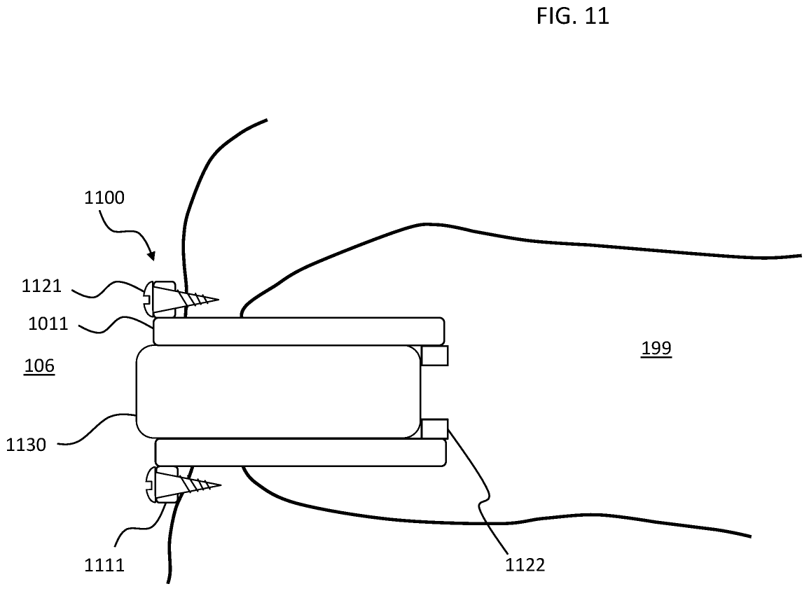

In some embodiments, bone cement or other adhesives can be utilized to permanently fix the body to the opening. In some embodiments, brackets can be utilized. For example, this can be seen in FIG. 11, where bracket 1111 is seen press fitted/interference fitted about the core of the body 1011 (collectively, the 1111 and the core 1011 make up the body—the core 1011 can be identical to the aforementioned body 1010 of FIG. 10 in some embodiments, and in others, can be different. For example, because of the bracket, a roughened surface/outer surface of the body 1010 that might be utilized to aid in the fixation might not be utilized with the core 1011 of FIG. 11, which presents an exemplary embodiment of an inner ear port device 1100 while in other embodiments, the roughened surface is utilized).

Here, the bracket has holes therethrough (not labeled) that receive one or more bone screws 1121 as seen. In an exemplary embodiment, the bone screws are what hold the body/fix the body to the passage. In an exemplary embodiment, a combination of the bone screws and an interference fit and/or a press fit with the passage through the bone 123 can be utilized. Note also that instead of bone screws and/or in addition to bone screws, bone cement or the like can be utilized, such as by way of example, by packing the bone cement between the flange 1111 and the surface of the bone 123 that faces the flange. It is noted that in at least some exemplary embodiments, the flange 1111 and the body 1011 are part of a monolithic component, which component can be turned on a lathe, from, for example, a thick-walled tube. Alternatively, the flange 1111 can be a washer type device or a ring type device which can be press fit or interference fitted on to a tube 1011.

In an exemplary embodiment, the inner ear port includes wires 849 or otherwise an electrically conductive material configured to conduct electrical current for the purposes of conducting an electrical signal. In an exemplary embodiment, the electrically conductive material can be lead wires. In an exemplary embodiment, the inner ear port includes electrodes that are connected to the lead wires 849.

It is noted that in an alternate embodiment, there are no definitive separate electrodes that are distinguishable from the leads. Instead, the electrodes could be the bare wires that extend into the passage 822. In this exemplary embodiment, the electrodes, whether they be distinct separate electrodes from leads or the ends of the leads, are electrically conductively exposed to the fluid within the cavity 199. In an exemplary embodiment, a potential between the electrodes and/or impedance between the electrodes can be measured to ascertain a latent variable that can be utilized to evaluate the perilymph within the cavity 199 for example. This could be used to determine the presence or absence of perilymph within the cavity 199 or otherwise a qualitative feature of the perilymph within the cavity 199, which can be utilized to evaluate the health of the inner ear and/or otherwise be utilized to determine whether or not a treatment regimen should be instituted, such as by way of example, providing a therapeutic substance utilizing the port device 800.

The wires 849 and the electrodes are passive components, at least if they are not attached to an active device. For example, say that the ends of the wires 849 (the ends in the middle ear) were utilized with a multimeter or some other device, but only when a surgeon or otherwise a healthcare professional accesses the middle ear to access the ends of those wires. The wires and the electrodes are passive functional devices that are utilized in conjunction with an active device. The multimeter is an active device.

In an exemplary embodiment, the implanted device is devoid of leads or wires or electrodes. In an exemplary embodiment, the implanted device is devoid of any conductive material that is utilized or otherwise configured to conduct electronic signals or otherwise conduct electrons, beyond the general structure of the body.

In an exemplary embodiment, the component supported by the body 810 is directly or indirectly releasably attached to the tissue interface portion and/or a portion of the device supported by the tissue interface portion, is devoid of active functionality. In an exemplary embodiment, the port device does not include any electronics, such as active electronic components. Herein, wires are not electronics, but wires forged or otherwise formed to have some feature beyond mere conduction of electrons such as, for example, a wire coil that forms an inductance coil, or a coil for an electromagnet, or a conductive element configured to have resistance to generate meaningful amounts of heat, is not a passive device. Thus, in this regard, in an exemplary embodiment, the port devices herein do not have any RF coils. Corollary to this is that in at least some exemplary embodiments, the port devices herein do not have any communication capabilities or otherwise include devices that provide communication (as differentiated from wires, which enable communication, but do not provide communication).

In an exemplary embodiment, the inner ear access devices herein are not smart devices. That is, by way of example, the devices are dumb and do not include or otherwise contain advanced components that would otherwise make the devices “smart.” In at least some exemplary embodiments, there is absolutely no control logic or control circuitry.

In an exemplary embodiment, there are no computer chip(s) and/or no microprocessors associated with the implant/device 800. In an exemplary embodiment, there is no onboard power source that is rechargeable or not rechargeable. For example, there are no onboard batteries associated with device 800. In an exemplary embodiment, there is no power source or powered components and/or powerable components (electrodes that are spaced so as to electrically communicate with each other are powerable components).

In some embodiments the inner ear port device according to at least some exemplary embodiments cannot monitor or otherwise measure any features related to the inner ear. Another device, such as those detailed above, are needed to work with device 800.

In this regard, in an exemplary embodiment, device 800 is a completely sensorless device and otherwise is a device that cannot stimulate tissue, at least in the absence of some other device that works with the device 800.

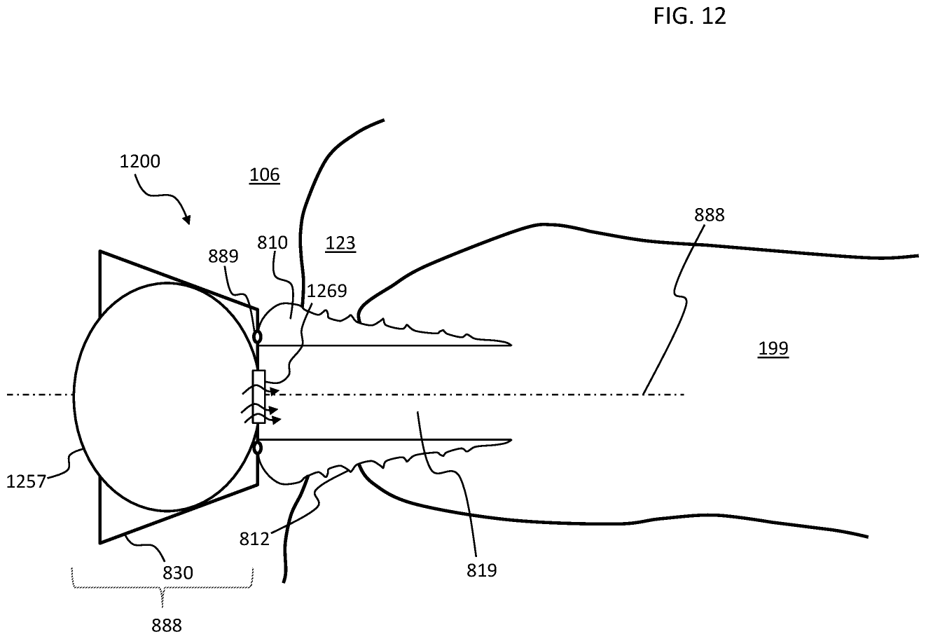

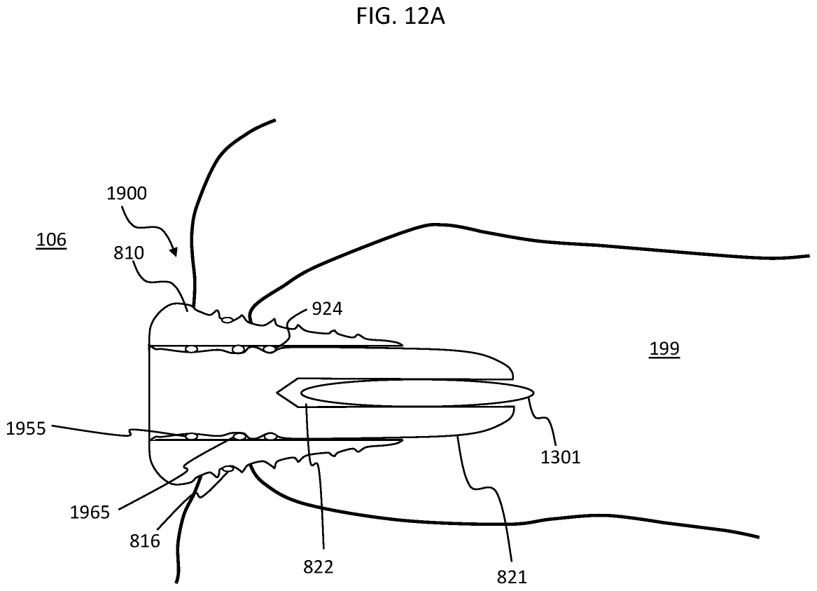

As noted above, in an exemplary embodiment, there is an inner ear device that is configured to enable resealable physical access from the middle ear into the inner ear through a passage. In the exemplary embodiment shown in FIG. 8, the passages passage 822, which is a passage through element 820, the details of which will be described below. But in some exemplary embodiments, the passage could be the passage shown that extends through the body 810 (in which element 820 is located). In an exemplary embodiment, the “head” of the inner ear port device 888 can be screwed onto and off of the body 810 by way of example. There can be seals on the head 888 that would seal the passage, and thus seal the environment of the middle ear cavity 106 from the environment of the duct 199. Accordingly, in an exemplary embodiment, there is a method of accessing the cavity 199 from the middle ear cavity 106, which includes the action of unscrewing the head 888 from the body 810 of the port device, passing a termination of a syringe through the passage 822 (which also means that the termination is passed through the passage established by the body 810 in which the element 820 is located, this can be passage 819 of FIG. 12, which is an exemplary embodiment without element 820) so that at least a portion of the termination is located within the extrapolated interior volume of the cavity 199, and then passing the therapeutic substance from the syringe through the termination into the duct 199, and then withdrawing the termination from the port device, and then reattaching the head 888 by screwing the head onto the body 810, thus sealing again the environment of the middle ear from the inner ear and vice versa. Accordingly, the head, with the utilitarian seal, enables resealable physical access from the middle ear into the inner ear through the passage. It is briefly noted that in the embodiment of FIG. 8, the action of removing the head 888 from the body 810 can be executed with the element 820 remaining in the passage 819 of the body 810, and relatively unmoved. (In an exemplary embodiment, conductive contacts can be used to place the lead portions of the head into electrical contact with the lead portions of element 820 (e.g., circular contact traces that accommodate for rotation of the head 888/misalignment when reinstalled/installed, that might exist)). Alternatively, and/or in addition to this, in an exemplary embodiment, as will be detailed below, element 820 can be structurally linked or otherwise fixed to head 888, and thus the action of removing head 888 from the body 810 can also result in the action of moving element 820 from the body 810. It is further noted that in some exemplary embodiments, such as where, for example, element 820 is a flexible body, such as, for example, a tube or sheath made of silicone, the flexible nature of the body 820 can be the feature that seals the passageway. For example, when the head 888 is screwed onto the body 810, the surface of the head facing the cavity 199 can come into contact with the opposite facing portion of elements 820, and thus compress the material of element 820, and thus establish the seal (where, for example, there is also a seal between element 820 and the inner passage of body 810).

It is again noted that while the embodiment of FIG. 7 presents a middle ear cavity 106 without ossicles, in an alternative embodiment, the ossicles are present and/or otherwise functional. This will be described in greater detail below. It is also noted that while the embodiment of FIG. 7 depicts the utilizations of the teachings detailed herein in the absence of another prosthesis and/or implant, such as a cochlear implant or a middle ear implant, it is to be noted that any disclosure herein of any embodiment associated with the port device corresponds to a disclosure of the utilization of such with any of the other devices/implants detailed herein unless otherwise noted. Accordingly, in an exemplary embodiment, with respect to the embodiment of FIG. 8, for example, there is also a cochlear implant electrode array that is located in duct 199 and/or in another duct of the cochlea and/or on another side of the cochlea in the same duct (e.g., the electrode array could be located in a portion of a duct that is the portion that is proximate to, for example, the oval window, and the port device could be located at the duct that is the portion of the duct that is proximate to, for example, the round window, the electrode array could be located in a portion of a duct that is the portion that is proximate to, for example, the round window, and the port device could be located at the duct that is the portion of the duct that is proximate to, for example, the oval window).

Further, it is noted that while some embodiments of the teachings detailed herein are utilized to treat the effects associated with implanting a component in the ear system of the recipient, such as by way of example only and not by way of limitation, providing anti-inflammatory substances and/or steroids and or NSAID's and/or non-steroidal anti-inflammatory drugs to the cochlea following a cochlear implant electrode array insertion, while other embodiments of the teachings detailed herein are not utilized per se with an implant. In this regard, the teachings detailed herein can be utilized to treat hearing problems irrespective of whether or not the recipient is utilizing a hearing prosthesis. By way of example only and not by way of limitation, in an exemplary embodiment, the teachings detailed herein can be utilized to treat a syndrome that is attacking the hair cells of the cochlea prior to the utilization of a hearing prosthesis. That is, the human has not received a cochlear implant, for example, and thus is being treated to preserve the hair cells to preserve as much hearing as possible. The future recipient or otherwise the human receiving the treatment may provide the therapeutic substances himself or herself by a self-used delivery device for example. That said, the teachings detailed herein can be utilized in isolation from any other prostheses or implant. It is also noted that the teachings detailed herein can be used in combination with conventional hearing aids. In this regard, the teachings detailed herein can be utilized to treat ailments associated with the hearing and/or balance system of a recipient that may or may not rise to the level of requiring an implantable and/or partially implantable hearing prosthesis, and the teachings herein can be utilized in combination with conventional hearing aids.

Some exemplary embodiments of the inner ear device are such that the body 810 corresponds to a first module of the implant, and the device includes a second module that is removably attached to the first module, the second module configured to enable the resealable physical access. In this regard, in an exemplary embodiment, head 888 can correspond to the second module, and can be the only second module such as, where, for example, there is no element 820. Such an embodiment is schematically depicted in FIG. 12. In this exemplary embodiment, a therapeutic substance container 1257 is located in the head 888, which container has a porous membrane 1269 in fluid communication with the interior of the container, wherein the opposite side of the porous membrane 1269 is in fluid communication with the interior of the cochlea 199 by way of passage 819. As is schematically depicted by the arrows, therapeutic substance contained in the container 1257 is transported from the interior of the container 1257 the passageway 819, and thus into the cochlea. In an exemplary embodiment, this is achieved via an overpressure in the container 1257, which overpressure exists at the time of implantation.

In the embodiment of FIG. 12, which depicts an exemplary inner ear port device 1200, there can be an O-ring seal 889 or the like as shown, which seal provides the sealing of the passageway 819 when the head 888 is screwed onto the body 810. The O-ring seal can be physically supported by the head 888 (meaning that the O-ring moves with the head) or can be supported by the body 810. A plurality of seals can be utilized. When head 888 is screwed on to body 810, the O-ring is compressed and the seal is formed in the traditional manner.

In this exemplary embodiment, referring back to FIG. 8 for example, element 820 is structurally attached to the head 888. When the head is unscrewed from the body 810, for example, and removed from the body 810, element 820 will move in a one-to-one relationship with the head 888. In an exemplary embodiment, the head 888 can be a housing established by titanium or a polymer or some other biocompatible material, where the head can include the electronics package 840. Indeed, in an exemplary embodiment, the head 888 can be a casting of a polymer in which the electronics package and the leads (the portion of the leads that are located in the head 888) are embedded. In an exemplary embodiment, the RF inductance coil can be embedded in the polymer. It is noted that embodiments can make ample use of hermetic enclosures, such as those that can be metal and/or ceramic (e.g., ceramic/metal braze feedthroughs). Thus, any disclosure herein of any component corresponds to a disclosure of an alternate embodiment or an additional embodiment where such components are hermetically isolated or otherwise hermetically sealed from the other components and/or the ambient environment. That said, in some embodiments, hermetic sealing is not necessarily needed her otherwise always utilized.

In an exemplary embodiment, the head 888 can be made out of biocompatible silicone.

The element 820 can be a silicone body or some other body that is made of biocompatible material that can correspond to a tube with a tapered end, although in other embodiments, the end is not tapered. Any one or more of the features detailed above with respect to the body 810 can be associated with the element 820 if there is utilitarian value with respect to doing so, providing the art enables such, unless otherwise noted. In an exemplary embodiment, element 820 can also establish a seal with respect to the interface between the outer surface of element 820 in the body 810, but element 820 also has passive functionality as detailed herein. Briefly, in an exemplary embodiment, a protrusion or plurality of protrusions 813 can be located on the outer surface of the element 820. This can have utilitarian value in, for example, an embodiment where the element 820 is made out of a flexible or an elastomeric material. That said, the outer surface can operate in a cork like manner or the like. And in this regard, in an exemplary embodiment, by way of example, with one side of the element 820 “wet” and another side of element 820 “dry,” or any other element that would fit into the passage 819 by way of example or any the other passages herein that have the wet/dry features, swelling properties of polymers or other types of material that swell in the presence of moisture can be utilized to provide a seal or otherwise obtain a seal.

In any event, in at least some exemplary embodiments, when element 820 is located in body 810, the only way that fluid can transfer from the cavity 106 to the cavity 199 and/or vice versa is through passage 822. As noted above, element 820 can be configured as a material that has elastic deformation capabilities, which can establish a seal between the head 888 and the element 820. That said, in an exemplary embodiment, such as where, for example, element 820 (or element 821—more on this below) is a titanium tube or a titanium body or some other metallic body, or some other structure that is relatively inflexible, where, for example, O-ring seal 924 as shown in FIG. 9 is utilized to seal the passage 819 in the body 810, and O-ring analogous to O-ring 889 can be located between element 820 and the head 888 (not shown in FIG. 9—FIG. 9 depicts an exemplary embodiment of a port 900 where there is no head 888, but in alternate embodiments, there can be a head (with or without element 930, the details of which will be described below). In this exemplary embodiment, the O-ring 924 is carried by the body 821. Conversely, in an exemplary embodiment, the O-ring 824 can be carried by the body 810. While only one O-ring as shown, in an exemplary embodiment, two or more of the rings or other type of seal, such as a gasket for example, can be utilized.

It is briefly noted that any of the features associated with element 820 can correspond to the features associated with element 821 and vice versa unless otherwise noted providing that the art enables such.

FIG. 12A presents an alternate embodiment of a device 1900 where a support body 821 is located in body 810, where support body 821 supports a container 1301 that contains a therapeutic substance, which substance can passively elute therefrom into the duct 199.

In an exemplary embodiment, the container 1301 contains a gas generator. In an exemplary embodiment, the implant 1900 is configured so that a water-based substance natural to the inner ear can enter the delivery apparatus. By “water-based substance,” this can be perilymph, or the water in perilymph for example. Along these lines, in an exemplary embodiment, a portion of the container 1301 can be of a porosity that permits water molecules to pass through, or can be of a porosity that permits perilymph to pass through. With respect to the former, the porosity would be such that water would disassociate from the remainder of the perilymph and travel through the porous barrier. With respect to the latter, perilymph could easily flow into the container. In the container 1301 could be a substance that generates gas when exposed to water and/or perilymph, etc. This would increase the pressure inside the container 1301, which would drive the therapeutic substance therein out into the duct 199. This can have utilitarian value with respect to providing a more thorough delivery arrangement that ensures or otherwise increases the amount of therapeutic substance that is ultimately delivered to the duct 199. That said, alternatively and/or in addition to this, the gas generating feature can be utilized to “time” delivery the therapeutic substance. By way of example only and not by way of limitation, this could be analogous to a cigarette bomb or the like, where a relatively standard amount of time will elapse before the perilymph or water can pass through the porous barrier, at least in sufficient quantities, to activate gas generation. Indeed, in an exemplary embodiment, there can be a barrier that dissolves in the presence of perilymph instead of or in addition to the porous barrier.

In view of the above, it can be seen that in at least some exemplary embodiments, there is an implant that is configured to passively initiate delivery of a therapeutic substance after a period of time after implantation, which delivery could be 0.1, 0.25, 0.5, 0.75, 1, 1.5, 2, 2.5, 3, 4, 5, 6, 7, 8, 9, 10, 11, 12, 13, 14, 15, or more hours, or days, or weeks, or any value or range of values therebetween in 0.05 hour increments after the container comes into contact with the perilymph. In an exemplary embodiment, the aforementioned time frames are time frames associated with the initial delivery of the therapeutic substance commencing. In an exemplary embodiment, the aforementioned time frames are the time frames before which, for example, no more than 1, 1.25, 1.5, 1.75, 2, 2.5, 3, 3.5, 4, 4.5, 5, 6, 7, 8, 9, 10, 15, 20, 25, 30, 35, 40, 45, 50, 55, 60, 65, 70, 75 or 80% or any value or range of values therebetween in 0.01% increments of the total available therapeutic substance that is delivered is actually delivered to the cochlea. The idea here is that it is possible in some embodiments that certain amount of therapeutic substance will be delivered initially, but the main thrust for the main delivery of the therapeutic substance can be timed in a delayed manner.

And note further that in an exemplary embodiment, the aforementioned gassing concept can be utilized to control the overall timing of the therapeutic substance delivery. For example, gas generation can take place based on a relationship (e.g. linear) of the amount of perilymph or water that works its way into the container. For example, because the rate of ingress of the water perilymph can be limited, gas generation could have a correlation with the ingress of the water perilymph, where in turn, pressure generation could have a correlation to the gas generation. If there is a correlation between pressure generation and the rate of delivery and/or the amount of delivery of the therapeutic substance, the rate and/or amount will have a correlation with the pressure generation.

Indeed, in an exemplary embodiment, there could be multiple pathways for perilymph or water to enter into the capsule, which pathways have varying time frames associated with the water perilymph reaching respective gas generation devices. This could provide for maintenance of pressure within the capsule, and/or could provide for a pulsatile increase in the pressure. The former would provide for a uniform or even delivery of therapeutic substance, while the latter would provide for a pulsatile delivery of the therapeutic substance. And the embodiments according to some embodiments can be configured so that this can occur over 0.1, 0.25, 0.5, 0.75, 1, 1.5, 2, 2.5, 3, 4, 5, 6, 7, 8, 9, 10, 11, 12, 13, 14, 15, or more hours, or days, or weeks, or any value or range of values therebetween in 0.05 hour increments.

Thus, it can be seen that in an exemplary embodiment, the delivery apparatus is configured so that an internal pressure of the delivery apparatus is increased due to the entry of a water-based substance, which pressure increase results in delivery from the delivery apparatus of the therapeutic substance into the inner ear.

And while the embodiments above have focused on the concept of gas generation, alternatively, and/or in addition to this, other pressure increasing devices can be utilized. By way of example only and not by way of limitation, a material that expands in the presence of water and/or perilymph could be utilized to increase the pressure. For example, a material that has a density that decreases in the presence of water, and thus as an overall volume that increases, could be utilized to increase the pressure in the capsule because the volume of this material increases, thus leaving less room for the therapeutic substance therein.

Any device, system, and/or method to passively control the delivery of therapeutic substance can be utilized in at least some exemplary embodiments.

In the embodiment of FIG. 12A, different sized O-rings 1965 and 1955 are used to seal between body 821 and body 810.

In this embodiment, passage 822 that contains container 1301 does not extend all the way through the body 821. Thus, there is only one passage from the middle ear into the inner ear (the passage in body 810).

Container 1301 can be glued or interference fitted into passage 822. In an exemplary embodiment, passage 822 could be shrink fitted around container 1301. Further, with respect to the embodiment shown, element 1301 can be, instead of a container, a solid body of a therapeutic substance that dissolves in the perilymph within the duct 199. Other types of therapeutic substance delivery devices can be utilized vis-à-vis implementation of element 1301.

Returning to the concept where the inner ear port includes a first and second module (where the first module is a tissue interface module, and the second module can be removed from the first module with the first module remaining in interface with tissue), it can be seen that in at least some exemplary embodiments, removal of the second module from the first module can establish the action of unsealing the established seal, to enable the physical access from the middle ear into the inner ear through the passage, whether that passage be the passage 819 and/or passage 822. And it is further noted that in some exemplary embodiments, such as where for example, the head 888 can be removed from the body 810 and the body 820, where the body 820 could also be removed from the body 810 (but may not be done so in some exemplary methods), and thus body 820 could be a third module by way of example, albeit this third module might not be one that is configured to enable the resealable physical access per se. Still, there can be utilitarian value with respect to enabling the removal of such to enable the ultimate replacement of body 820 to upgrade the port device and/or to otherwise address a wear scenario.

A male-female coupling relationship can be used, such as, in some exemplary embodiments where the head 888 is snapped coupled to the body 820 and/or to the body 810.

And it is noted that in at least some exemplary embodiments, while body 820 is presented as a separate component from body 810, in some exemplary embodiments, one or more of the features of the body 820 are part of the body 810. Indeed, in an exemplary embodiment, the structure that is identified as body 820 can instead be part of an integral body 810. To be clear, we are not disclosing that there is meaninglessness between the two bodies. All we are doing is describing that in the interest of textual and schematic economy, that one or more of the features of the body 820 could be present with the body 810. By way of example only and not by way of limitation, there may be body 820, and instead, body 810 encompasses everything that is shown with respect to body 820, except the gap that is shown between the two that is associated with the protrusion 813 (which would not be there if, for example, everything was part of a single component).

In any event, just briefly, in an exemplary embodiment, the combination of head 888 and body 820 can correspond to the second module, and this can operate as a plug type device, and analogous to a cork where the outer surface of body 820 and/or a seal on the outer surface of body 820 provides the sealing features when the body 820 is an passage 819, and then upon the removal of such, which can be executed by simply pulling head 888 away from body 810, and thus pulling body 820 out of passage 819, such establishes a passageway to enable the physical access from cavity 106 to cavity 199.

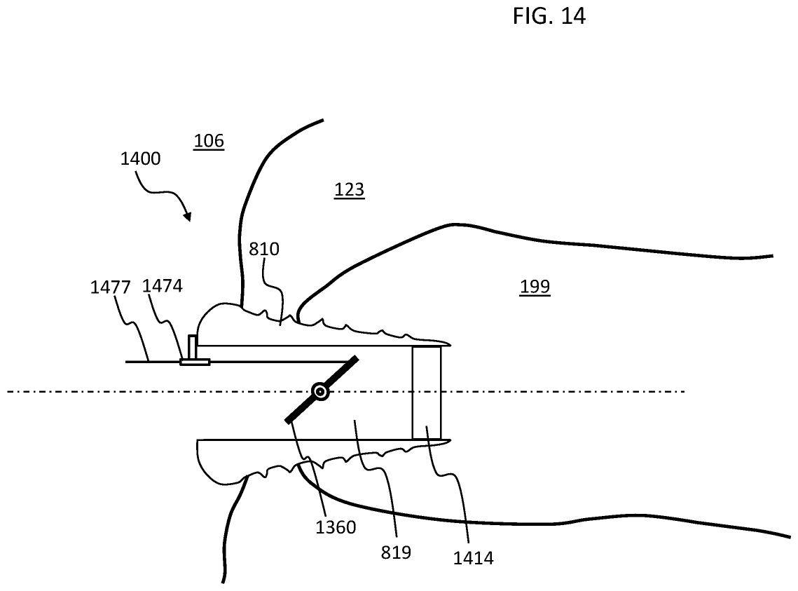

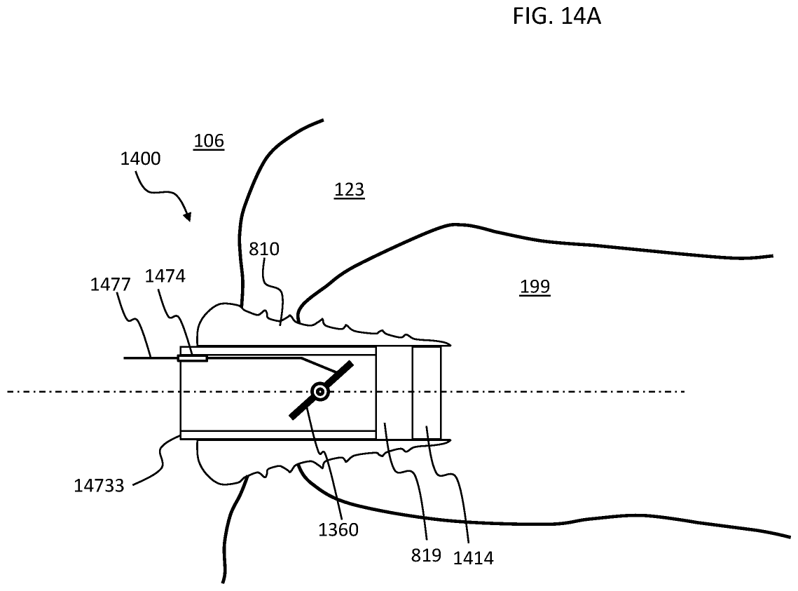

In an exemplary embodiment, the inner ear port device establishes a fluid valve between fluid of the inner ear and an outside of the inner ear. By way of example only and not by way of limitation, FIG. 14 depicts an exemplary embodiment where, for example, a butterfly valve 1360 is positioned within the passageway 819. In an exemplary embodiment, the butterfly valve is mechanically coupled to linkage 1477 that extends into the middle ear cavity 106 as shown, or otherwise can be accessible from the middle ear cavity, so that the linkage can be manipulated by a surgeon or a healthcare professional to open, close and/or adjust the valve 1360. By way of example only and not by way of limitation, element 1477 can be an elongate beam with a circular cross-section that passes through a hollow cylinder 1474 that establishes a slight interference fit or otherwise a slight resistance that with element 1477, where cylinder 1474 is supported on body 810 by a cantilever beam as shown. The surgeon could utilize a tweezers or a forceps to pull and/or push on the beam 1477 two vary the position of the butterfly valve 1360. In this arrangement, there is no actuator, and certainly nothing that is powered to operate or otherwise control the location of the valve. The valve is completely passive.

And to be clear, while the embodiment of the valve shown in FIG. 14 shows the valve is being mounted to body 810, in an alternate embodiment, the valve can be part of a second module such as any of those detailed herein and/or variations thereof, and the valve can be mounted thereto. This is seen in FIG. 14A, where hollow cylinder 14733 supports the linkage 1477 and the valve 1360, where the hollow cylinder can be secured to the body 810 in a manner concomitant with any of the second modules/second components that interface with the body 810 as detailed herein.

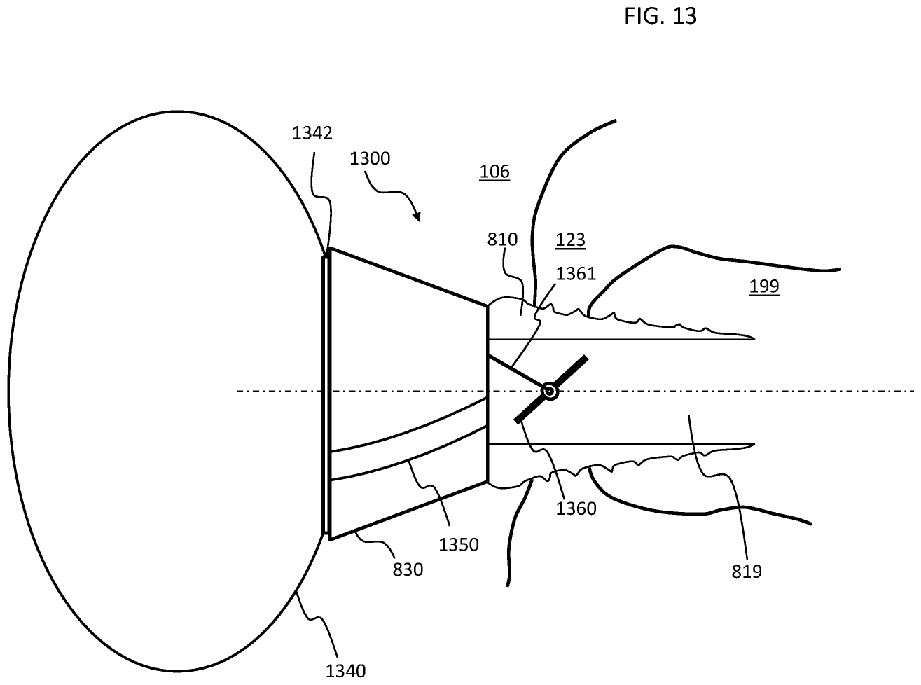

FIG. 13 presents an exemplary embodiment where by way of example only and not by way of limitation, there is a therapeutic substance reservoir 1340, which can be, for example, a stretched elastomeric material in the form of a flexible container, which could have balloon-like qualities, where, the inside is over pressured relative to the pressure within the cochlea and/or within the middle ear, and thus the therapeutic substance contained therein, which can be a fluid (liquid and/or gas) thus has a predisposition to exit the reservoir 1340. The reservoir 1340 is releasably connected to the head 888 by coupling 1342 which can be configured to enable the removal and installation/attachment of the reservoir to the head 888, or can be permanently fixed thereto (the removable feature/releasably connection feature enables the reservoir 1340 to be replaced, such as after the reservoir is depleted, for example). Here, there can be a conduit 1350 that extends from the reservoir 1340, more accurately, from an opening in the reservoir, or otherwise a port in the reservoir, that enables fluid communication from the interior of the reservoir 1340 through the head 888 to the passageway 819. In this exemplary embodiment, when the valve 1360 is closed, the therapeutic substance, which is under pressure owing to the nature of the reservoir 1340, cannot travel from the proximal side of the valve to the distal side of the valve, and thus cannot enter the cavity 199, and thus cannot comingle with the perilymph therein. When the valve 1360 is open, therapeutic substance can travel into the cavity 199, and thus commingle with the perilymph therein. The valve 1360 is supported by strut 1361, which is connected to the head 888. In an exemplary embodiment, where the head is removable from the body 810, removal of the head will also bring the valve 1360 out as well, and thus the only part that would be permanently fixed is the body 810.

Consistent with the teachings above, there can be linkage that is connected to the valve 1360 so that the valve can be manipulated from the middle ear.

In an exemplary embodiment, there are no valves in the port and in some embodiments, there are no valves that are able to be controlled directly by human. Control would be due to, for example, physical structure with a preordained outcome, such as, for example, a spring-loaded ball valve that opens when the pressure difference overcomes the force on the spring. In an exemplary embodiment, a relatively small example of a valve can be a slit in a tacky polymer, such as, for example, silicone, which is normally sealed by the tacky nature of the silicone, but opens at a given pressure.

Returning back to FIG. 14, in this exemplary embodiment, the therapeutic substances can be delivered in a more traditional manner by filling or otherwise inserting a gel into the middle ear cavity. The gel will flow naturally to the passage 819, and thus to the valve 1360. The valve can be controllably opened and closed, to enable the therapeutic substance to reach the distal side of the valve, such as using the linkage detailed above. Shown in FIG. 14 is a barrier 1414 that has a structure, such as by way of porosity, that enables the therapeutic substance to defuse or otherwise flow from the passage 819 into the cavity 199, but which prevents the perilymph in cavity 199 from flowing outward into passage 819. In an exemplary embodiment, element 1414 can be a membrane. In an exemplary embodiment, element 1414 can be a device where the porosity thereof can be changed or otherwise be controlled. By way of example only and not by way of limitation, in an exemplary embodiment, an electric current can be provided to element 1414 (using a device that is not part of the implant) that reduces the porosity thereof or otherwise establishes a porosity thereof when the current is applied, or vice versa. In an exemplary embodiment, the gel or other excipient or some other substance applied in the middle ear could control the porosity. Any device, system, or method that can enable the transfer of therapeutic substance from passage 819 into the cavity and can prevent perilymph, or at least prevent medically significant amounts thereof from flowing in the other direction, into passage 819, can be utilized in at least some exemplary embodiments. To be clear, as can be seen, the proximal end of the passage 819 is open to the middle ear cavity 106 so that the gel or the like that is inserted into the middle ear cavity can reach the valve 1360, and thus the barrier 1414.

In an exemplary embodiment, the membrane or other barrier between the middle ear and the inner ear can be made of a material that reacts or otherwise transforms when exposed to certain chemicals. By way of example only and not by way of limitation, a material that could swell dramatically or otherwise shrink dramatically could be utilized. Material could be placed into the passageway and secured on one side of the passageway. When exposed to the chemical, the material could shrink and thus permit passage of a therapeutic substance through the passageway of the port. In an exemplary embodiment, the aforementioned gels or the like could carry this chemical, and when the gel is placed in the middle ear, the chemical comes into contact with the material that transforms the presence of the chemical. After this, the therapeutic substance enters the inner ear through the passageway, and then eventually, the membrane altering compound/chemical/substance evaporates or otherwise is sufficiently diluted that the membrane where the material in the passageway transforms back to its original state, thus resealing the passageway.

In at least some exemplary embodiments, the inner ear port device is configured to enable flow of perilymph or other inner ear fluids from the inner ear out of the inner ear. In an exemplary embodiment, there can be utilitarian value with respect to having the perilymph, for example, mixed with a substance within the reservoir. In an exemplary embodiment, one reason to do this could be for diagnostic purposes. Still further, in an exemplary embodiment, embodiments can include the exchange of perilymph within the cochlea with artificial perilymph located outside the cochlea, and thus perilymph in the cochlea can be removed, and thus the perilymph can be transferred from inside the cochlea to outside the cochlea through the inner ear port device. The aforementioned valves detailed herein can enable such in at least some exemplary embodiments.

Thus, as seen above, some exemplary embodiments of the inner ear port device includes a valve that enables the control of the fluid flow from the outside of the inner ear in general, and the cochlea in particular to the inside of the inner ear in general, and the cochlea in particular, and/or the other way around. This valve may include the linkage detailed herein.

In an exemplary embodiment, the inner ear device is configured to enable control of the delivery of therapeutic substance into the inner ear, such as by way of example only and not by way of limitation, starting and/or stopping the movement thereof into the inner ear. In an exemplary embodiment, this can be achieved by, for example, controlling the valve. In an exemplary embodiment, the valve can be the butterfly valve or flapper valve detailed above (any valve herein can be a butterfly valve or a flapper valve, and embodiments where one is disclosed corresponds to a disclosure of the other), while in other embodiments, other types of valves can be utilized, such as by way of example only and not by way of limitation, a sphincter valve and/or a check valve/one-way valve and/or a ball valve, etc. Any device, system, and/or method that can enable the control of transportation of the therapeutic substance, including the stopping and starting of the substance, can be utilized in at least some in some embodiments.

In an exemplary embodiment, there is no autonomous and/or no semi-autonomous characteristics of the implant.

In an exemplary embodiment, as differentiated from, for example, the arrangement of FIGS. 5 and/or 4, which, as noted above, are not part of the invention, but provide teachings that can be utilized to implement some aspects of the invention, all of the componentry associated with the inner ear device is located within a volume of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 90, 100, 125, 150, 175, 200 or 300 or 400 or 600 or 800 or 1000 mm³ or any value or range of values therebetween in 1 mm³ increments. In an exemplary embodiment, the aforementioned volumes are established by a cube volume, or a volume established by rectangular sides (again, it is within—the device need not be a cube—this can be analogous to shipping volume specifications for a box or container) where the largest straight dimension of the side is 0.5, 0.6, 0.7, 0.8, 0.9, 1.0, 1.5, 2, 2.5, 3, 4, 5, 6, 7, 8, 9, 10, 15 or 20 mm or any value or range values therebetween in 0.1 mm increments.

In an exemplary embodiment, the entire device of the inner ear port is located within 0.5, 0.6, 0.7, 0.8, 0.9, 1.0, 1.5, 2, 2.5, 3, 4, 5, 6, 7, 8, 9, 10, 15, or 20 mm, or any value or range of values therebetween in 0.1 mm increments of a natural inner ear cavity.

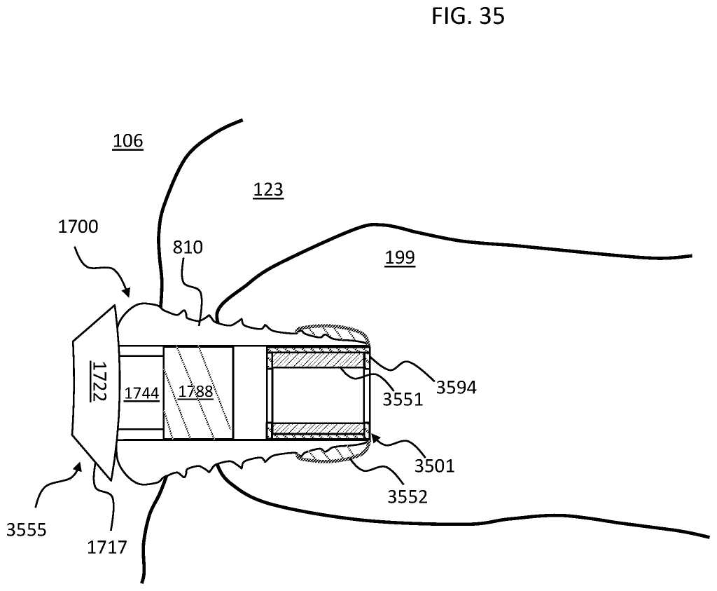

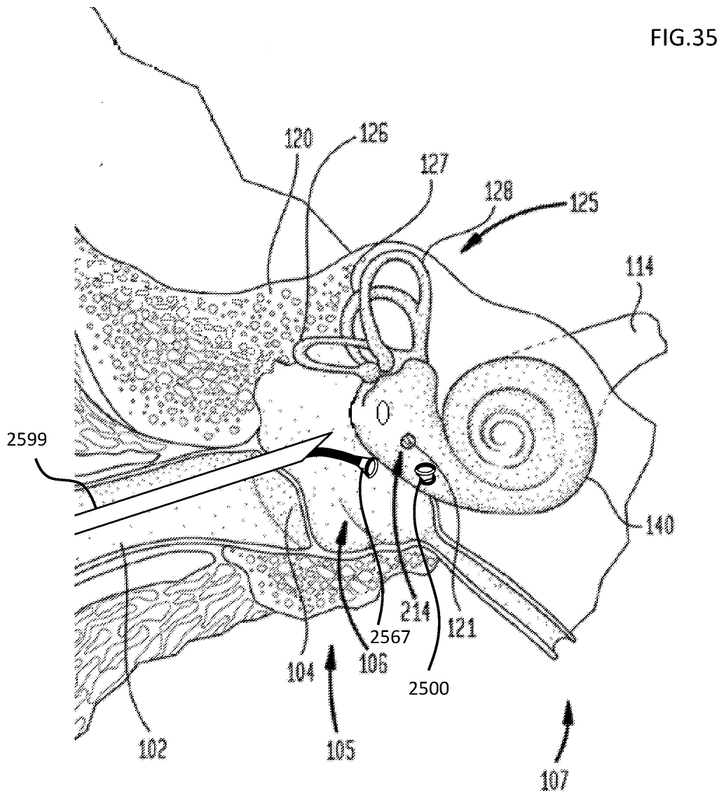

In view of the above, it can be seen that in an exemplary embodiment there is a device, such as an implantable device, including a tissue interface portion, such as body 810 of FIG. 8, configured for securement to tissue of and/or proximate an inner ear of a human and provide a structurally stable passage, which can be a long term passage, from outside the inner ear to inside the inner ear (passage 819, for example). The device also includes a component (e.g., the assembly including element 820 and head 888) releasably attached to the tissue interface portion and/or a portion of the device supported by the tissue interface portion (e.g., there could be an intermediate device between the two). This component is a passive component. By way of example only and not by way of limitation, a drug-eluting capsule can be located in passageway 822. Alternatively, the passive component can be the apparatus of head 888 of FIG. 12, where the therapeutic substance diffuses through barrier 1269. In an embodiment, the device is configured to enable the component to be removed from the tissue interface portion when the tissue interface portion is removably permanently fixed to the barrier establishing the inner ear of a human, and the component at least partially seals the passage and provides one or more passive features. As will be detailed below, the component can completely seal the passage. In other embodiments, the device is configured to enable the component to be removed from the tissue interface portion when the tissue interface portion is removably permanently fixed to the barrier establishing the inner ear of a human, and the device also includes a removable seal apparatus configured to unsealably seal the passage (this is described below for example with respect to FIG. 35).

By “passive component,” it is meant just that—it is a component that is characterized as a passive component, as distinguished from, for example, an active component. If the component also has a functionality that has an active feature thereof, it is not a passive component. By way of example only and not by way of limitation, say, for example, that there was a sensor that was supported by element 820 of FIG. 8, and element 820 also supported a therapeutic drug container configured to passively elute the therapeutic substance there out, that would not be a passive component because the component would be characterized as having active functionality. This would be a component that is an active component that also has a passive device. In any event, in this exemplary embodiment, the device is configured to enable the passive component to be readily removed from the inner ear barrier tissue interface when the inner ear barrier tissue interface apparatus is permanently fixed to the barrier establishing the inner ear of a human.

Thus, in view of the above, it can be seen that in an exemplary embodiment, the passive component that is attached to the tissue interfacing portion is a therapeutic substance delivery device.

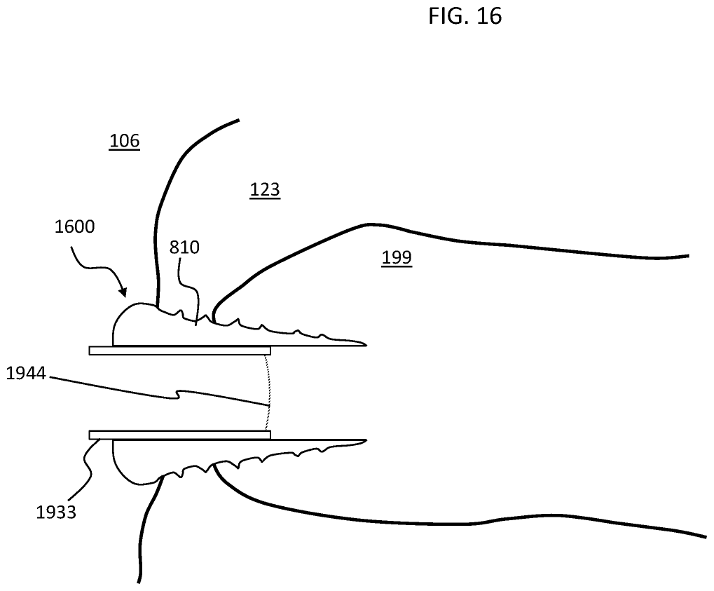

In an exemplary embodiment, the passive component is an artificial round window (or oval window). FIG. 16 presents an exemplary inner ear device, prostheses 1600, that includes the body 810 detailed above, into which is screwed or otherwise fixed, tube 1933 (the cross-section of which is shown without backdrops, as is the case with all of the cross-section shown a hearing). Shown at the distal end of tube 1933 is a diaphragm 1944 or membrane 1944. In combination, the tube 1933 and the diaphragm 1944 collectively can correspond to a second module, where the body 810 can correspond to the first module. Collectively, tube 1933 and the diaphragm 1944 correspond to a passive component.

In an exemplary embodiment, the structural makeup of the diaphragm or membrane 1944 is configured to duplicate or otherwise mimic or otherwise provide for the functionality of a natural round or oval window, depending on the arrangement. In the embodiment depicted in FIG. 16, the tube 1933 is configured to be removed and replaced from the body 810. This can enable the ability to replace a defective or otherwise worn artificial round window or oval window, by way of example. The membrane or diaphragm is located at a distal end of the tube 1933 so that the actions associated with attaching the membrane or diaphragm to the interior of the tube can be more easily executed relative to that which would be the case if the diaphragm or membrane was further in the tube. In an exemplary embodiment, the diaphragm or membrane can be located at the very end. That said, in an alternative embodiment, the diaphragm and/or membrane can be located anywhere in the interior of the tube. The diaphragm and/or membrane can also be located on the proximal end of the tube 1933 or proximate the proximal end of the tube 1933

It is briefly noted that the diaphragm or membrane 1944 is shown in a flex state. This is done for purposes of illustration only. In an exemplary embodiment, the relaxed state of the membrane or diaphragm 1944 is relatively planar, although in other embodiments, a pre-curved shape could be utilized for the relaxed state.

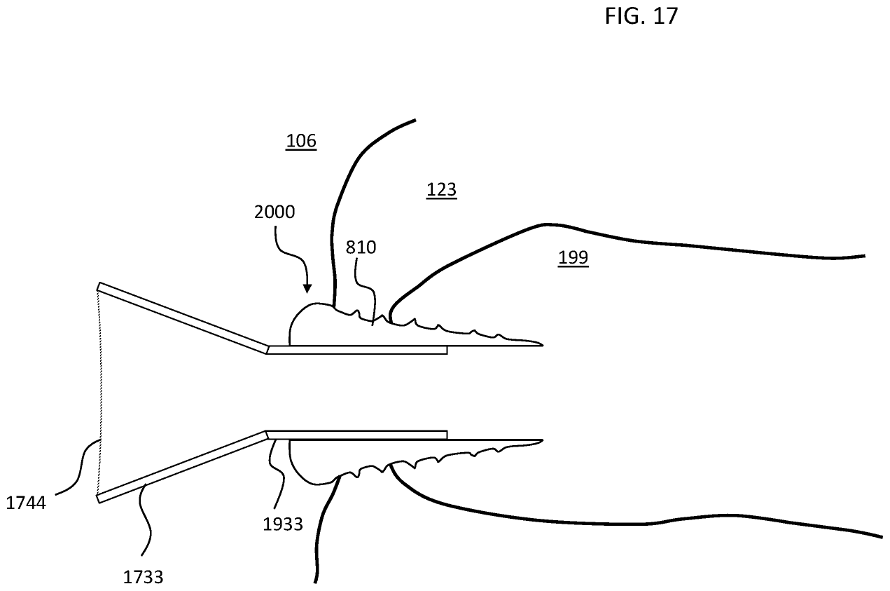

FIG. 17 presents another exemplary embodiment of an artificial round window or oval window prosthesis 2000 according to an exemplary embodiment. Here, for whatever reason, the size of the exemplary diaphragm or membrane 1944 of the embodiment of FIG. 16 is limited or otherwise the structure of the diaphragm or membrane will not enable sufficient replication or mimicking of the performance features of a natural round window or oval window. Alternatively, and/or in addition to this, there can be utilitarian value in purposely not mimicking or otherwise replicating the exact performance features of a natural round window or oval window. FIG. 17 provides an embodiment that addresses such scenarios. Here, the tube 1933 is attached to a funnel 1733, by some form of attachment, in some embodiments, elements 1933 and 1733 are part of a monolithic component (a tube having a diameter of body 1933 is flared out and plastically yielded to achieve the funnel like shape of 1733). In an exemplary embodiment, a funnel structure is utilized, but in an alternative embodiment, the transition from tube 1933 to the wider area can be more abrupt (it could be that the area established by the structure 1733 is a rectangular area instead of a trapezoidal area-any shape that can enable the teachings detailed herein can be utilized). In any event, the concept here is that by widening the cross-sectional area (the area lying on a plane normal to the longitudinal axis of the prosthesis) of the location where the membrane or diaphragm 1744 is to be attached to the structure that establishes the fluid tight body that extends from the body 810, a membrane or diaphragm 2044 with a larger surface area than that which would otherwise be the case if the membrane was located inside the passageway through the body 810 can be achieved. This can have utilitarian value with respect to potentially utilizing a stiffer membrane or diaphragm relative to a natural round window or oval window, but by increasing the surface area relative to that which would be the case of the diaphragm or membrane was located in the passageway of body 810, the performance features of the natural round window or oval window can in some embodiments be more closely matched (including exactly matched or effectively exactly matched). That is, by way of example only and not by way of limitation, there could be a scenario where the materials available or otherwise desired for the establishment of the artificial round window or oval window would not be able to replicate the performance of those windows if located in the passageway of the body 810, where the size of the passageway is governed by the dimensions of the body 810, which are governed by the dimensions of the passageway that is established from the middle ear cavity to the inner ear cavity.

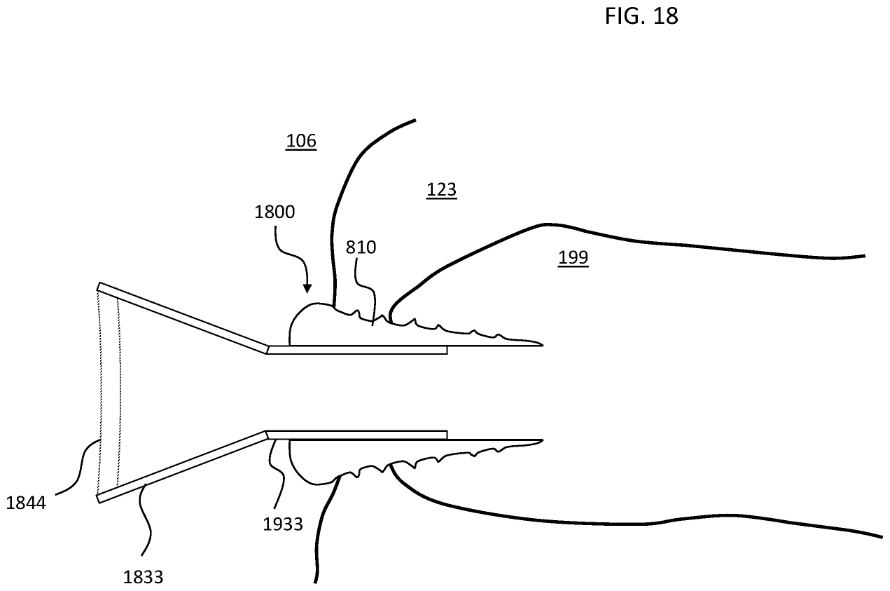

Corollary to this is that in some embodiments, it is specifically desired to utilize a material that does not necessarily function on a per area basis in a one-to-one relationship with the natural tissue of the round window or the oval window. By way of example only and not by way of limitation, in an exemplary embodiment, the material that is utilized to establish the artificial window is material that is also utilized as a self-healing septum. That is, in this regard, it is potentially thicker than that which would be the case if a self-healing septum feature was not desired with respect to a performance factor of the artificial window. FIG. 18 depicts the inner ear prosthesis 1800 according to this exemplary embodiment, where a self-healing septum 1844 is shown. The size of the self-healing septum enables the performance features of the round window or oval window even though the material properties are different than that of the natural round window or oval window. The septum 1844 is configured to permit repeated puncturing and subsequent healing by a termination of a syringe, while also providing for the functionality of an artificial round window or oval window.

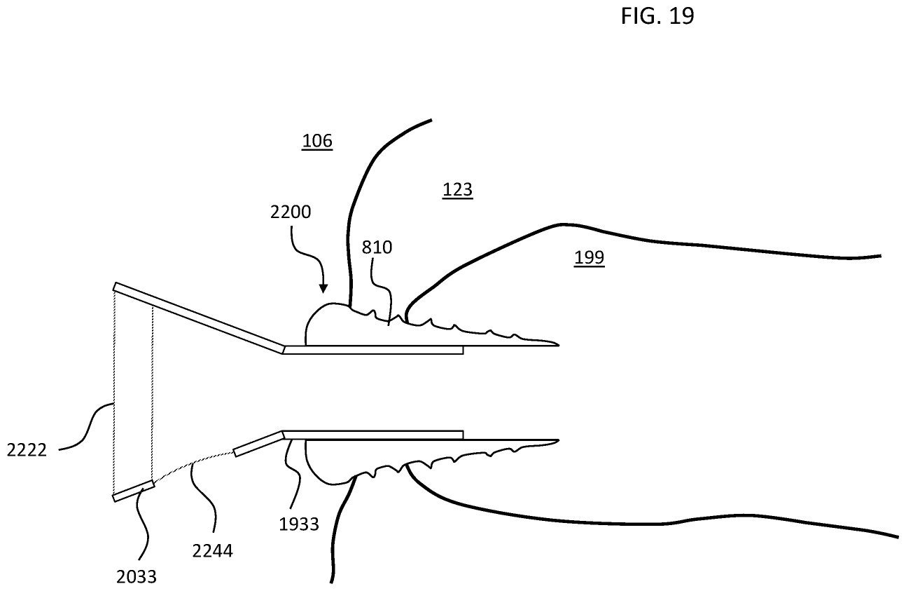

The above said, FIG. 19 depicts another exemplary embodiment of an inner ear device 2200, where there is a septum 2222 that is self-healing (more on this concept below), and an artificial round window 2244. Here, the septum 2222 is configured to be substantially inflexible, or effectively inflexible relative to the performance features of the round window, but still providing the ability to have repeated access to the interior of the inner ear, or at least provide repeated access to a space that is in fluid communication with the inner ear. The artificial round window 2244 provides the functionality of the natural round window. And while the fall shape embodiment is presented in FIG. 19, in other embodiments, the rectangular shape can be utilized, and thus further protecting the artificial round window 2244 from the sharp end of the termination of the syringe, because the artificial round window 2244 can be in the “shadow” of the wall that supports the artificial round window with respect to the longitudinal axis.

As is the case with respect to the embodiment of FIG. 16, the embodiment of FIG. 19 (and the embodiments of the intervening figures, for that matter) enables the artificial round window or oval window to be replaced as needed while maintaining the tissue interface 810 in place.

Consistent with the teachings detailed above, in an exemplary embodiment, the tube that supports, directly or indirectly, the artificial round window or the artificial oval window, can be screwed into the body 810 owing to screw threads located in the body 810 and the tube. In an exemplary embodiment, the tube is press fitted or interference fitted into the passageway in the body 810.

Embodiments of the inner ear prosthesis that replicate the performance of the round window or oval window can be located at the locations or former locations of those tissues. Alternatively, in an exemplary embodiment, the inner ear prosthesis that replicate the performance of the round window or oval window can be located at other locations, such as at locations in a cochleostomy through the bone that establishes the boundary between the middle ear in the inner ear further up the scala. This is consistent with any of the teachings detailed herein with respect to any of the inner ear prosthesis detailed herein.

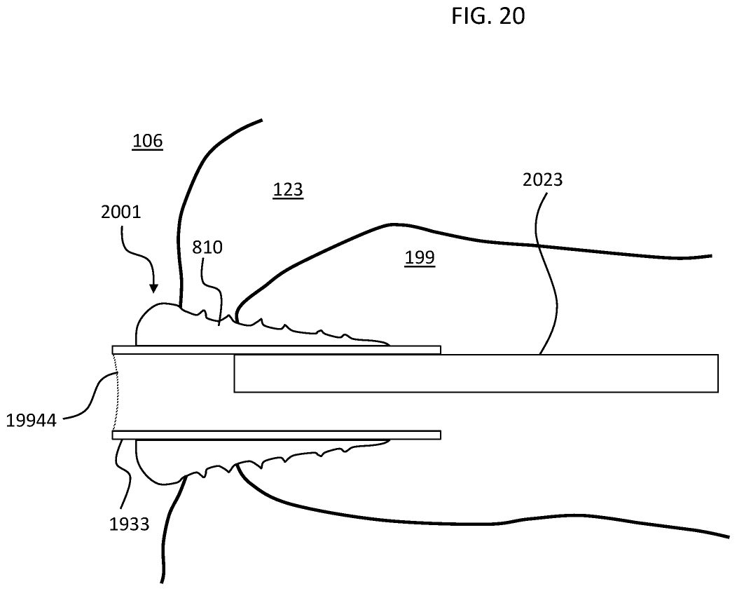

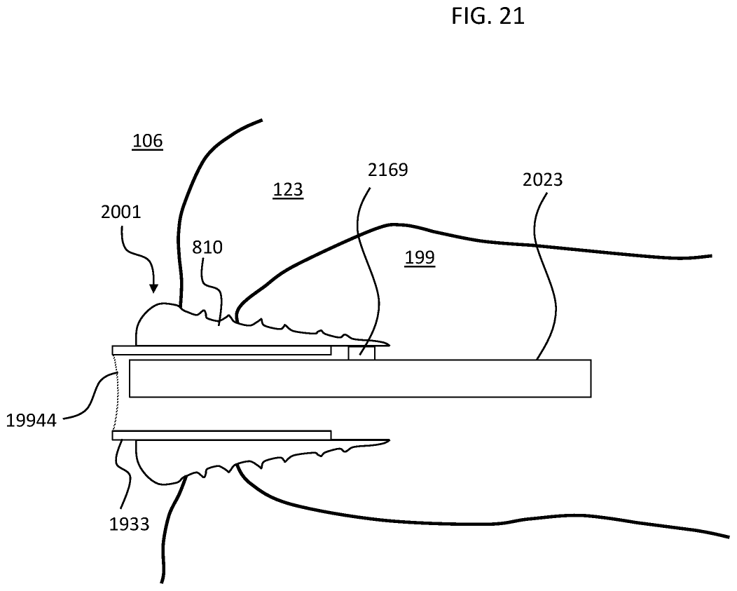

FIG. 20 presents another exemplary embodiment of an inner ear device 2001. Here, there is the first 810, the tissue interface body, which can correspond to the body detailed above. Here, the second module is established by a tube 1933, and there is an artificial round window or oval window 19944 as seen. This is an exemplary embodiment where the artificial round window or oval window is located at the proximal end of the prostheses, and embodiment briefly mentioned above. This exemplary embodiment of implant 2001 further includes a therapeutic substance delivery submodule 2023 of the second module. In this exemplary embodiment, element 2023 can be a reservoir that contains a therapeutic substance, where the therapeutic substance is configured to defuse through the outer wall thereof (in a passive manner, thus rendering the second module a passive component—if it was by an active pump for example, the second component would not be a passive component—that said, if element 2023 was a standalone module, that was for example, attached to the passage of body 810 by support strut 2169, for example, as shown in FIG. 21, then that would render the tube 1933 a passive component even though there is a separate active component (element 2023 could be an active sensor or a stimulation device, or have a drug pump that pumps drug into the cochlea) attached to body 810—note that the arrangement of FIG. 21 could also be for a passive module 2023). In an exemplary embodiment, element 2023 can be a body of the therapeutic substance in a solid configuration, and the solid configuration will dissolve when exposed to the perilymph. By sizing in dimensioning element 2023 relative to the inner diameter of tube 1933, the functionality of the artificial round window or oval window 1944 can be maintained so that the pressure changes in movement of the fluid within the cochlea can adequately reach the membrane or diaphragm 1944.

In this exemplary embodiment, element 2023 can be a reservoir that contains a therapeutic substance, where the therapeutic substance is configured to defuse through the outer wall thereof. In an exemplary embodiment, element 2323 can be a body of the therapeutic substance in a solid configuration, and the solid configuration will dissolve when exposed to the perilymph.

In an exemplary embodiment, the port device is completely unrelated to any function in the auditory system. In an exemplary embodiment, the only function related to the auditory system is the pressure compensation associated with providing an artificial round window. In an exemplary embodiment, the port device is related to a function of the auditory system, such as where the port device is utilized with the cochlear implant electrode array as noted above.

In an exemplary embodiment, the round window is completely replaced with the inner ear port device. In an exemplary embodiment, the round window is artificially ossified by a surgeon or other healthcare professional, on purpose, and then the functionality thereof is entirely replaced by the inner ear port device according embodiments herein.

Embodiments thus include an implant that can mimic the natural round window function. In an exemplary embodiment, utilizing a statistical average (mean, median, and/or mode) performance feature of a round window for a 50 percentile human born Mar. 23, 1960, in the United States of America, as the baseline, the ability to compensate for pressure fluctuations within the cochlea for sound captured by the tympanic membrane at 1000 Hz at 60, 65, 70, 75, 80, 85, 90, 95, or 100 dBs, or any value or range of values therebetween in 1 dB increments, is at least 70, 75, 80, 85, 90, 95, 96, 97, 98, 99, or 100%, or any value or range of values therebetween in 1% increments of the compensation that is provided by the aforementioned statistical average round window.

In an exemplary embodiment, the artificial round window provides a system that is configured to balance pressure within a cochlea at rates of at least 200, 300, 400, 500, 600, 700, 800, 900, 1000, 1250, 1500, 1750, 2000, 2500, 3000, 4000, 5000, 6000, 7000, or 8000 Hz, or any value or range of values therebetween in 1 Hz increments.

And it is noted that in at least some exemplary embodiments, a plurality of artificial round window devices can be utilized. One window can be tuned or otherwise configured to accommodate lower ranges of frequencies, and another could be tuned or otherwise configured to accommodate higher ranges of frequencies and/or medium ranges of frequencies, and the other could be tuned or otherwise configured to accommodate higher ranges of frequencies.

It is noted that while the embodiment of FIG. 19 is presented with element 2222 in combination the artificial round window or the artificial oval window, in other embodiments, element 2323 can be a standalone component (e.g., a solid rigid wall can be located where the diaphragm or membrane 2244 is located, which solid rigid wall closes the volume to prevent perilymph from escaping into the middle ear cavity).

In some embodiments, the walls of the tube 1933 can be configured to dissolve or otherwise elute a therapeutic substance into the perilymph that comes into contact with the tube 1933. In this regard, the inner ear device can include a module that includes a bulk wall containing a therapeutic substance.

In an exemplary embodiment, as noted above, the artificial round window can replace the existing round window, and thus the prostheses can be located in the round window niche. The artificial window, or any of the other prostheses detailed herein, can be placed in the promontory or other appropriate location. In an exemplary embodiment, the artificial round window is actually smaller than the natural round window. For example, the surface area of the artificial round window can be less than 70, 60, 50, 40, 30, or 20%, or any value or range of values therebetween in 1% increments than the natural round window that it replaces.

Embodiments can enable repeated sealingly access from the middle ear to the inner ear through a sealable passage in the prostheses. This can be achieved, by way of example, by the head 888 that can be screwed onto the body 810 as detailed above. Alternatively, and/or in addition to this, in an exemplary embodiment, this can be enabled by, for example, the aforementioned flapper valve of the embodiment of the implant 1400 detailed above. Embodiments can also utilize, for example, a self-healing septum. In this regard, in an exemplary embodiment, the passive component at least is indirectly releasably attached to the tissue interface portion and/or a portion of the device supported by the tissue interface portion can include a self-healing septum.

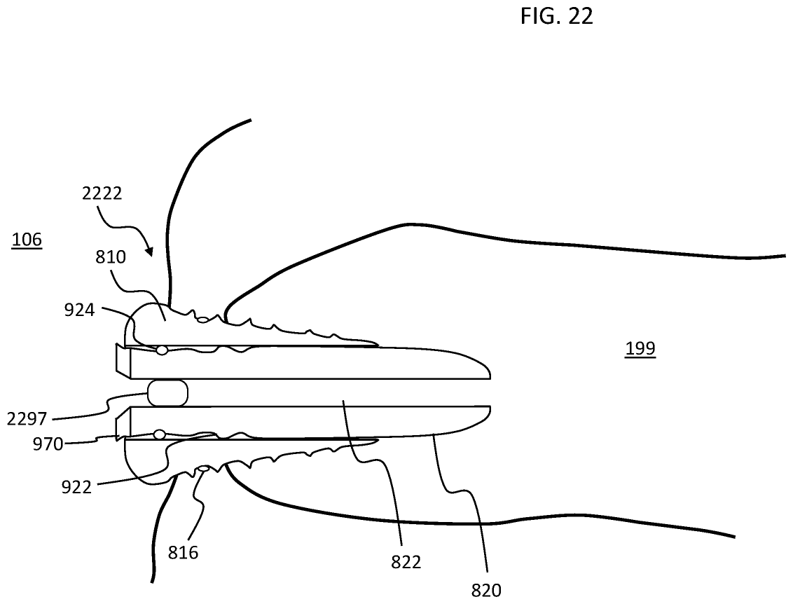

FIG. 22 presents implant 2222, which includes septum 930 which is configured to self-heal. Septum 930 seals the passageway 822 and otherwise establishes a barrier between the cavity of the inner ear 199 and the cavity of the middle ear 106. In an exemplary embodiment, septum 930 is configured to receive and otherwise permit a termination of a syringe, such as that of a hypodermic syringe, to pass therethrough in a manner analogous to or otherwise the same as liquid medical containers that include septums (self-healing septums) that enable the termination the past therethrough to access the liquid therapeutic substance in the container. In at least some exemplary embodiments, any device, system and/or method that will enable repeated sealingly access from the middle ear to the inner ear can be utilized in some embodiments.

Also, seen is that there is a cap 970 attached to the end of element 820. This cap provides a chamfered surface to guide the tip of the termination. That is, this can have utilitarian value with respect to guiding a terminal of a syringe to the passage through element 820.

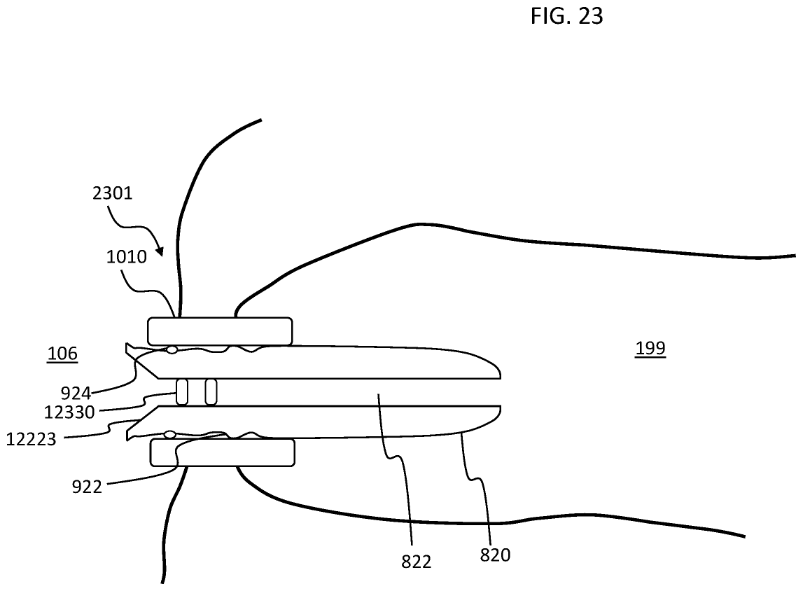

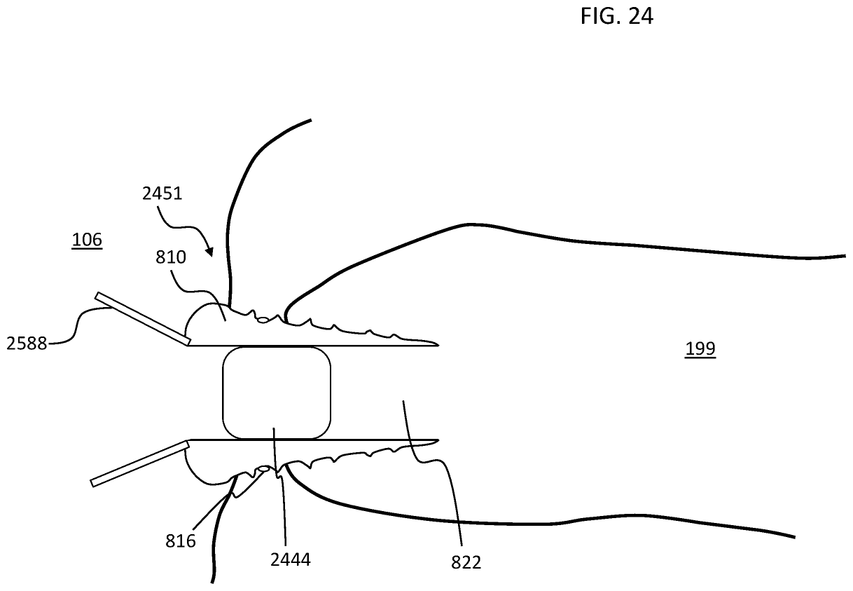

FIG. 23 presents an implant 2301, it can be seen that in an exemplary embodiment, a plurality of septums 12330 can be utilized. This can provide redundancy and otherwise provide an additional barrier to prevent transfer of unwanted substances from the middle ear into the inner ear and/or provide redundancy to prevent the transfer of substances from the inner ear to the middle ear. Also, implant 2301 can be seen to include a funnel 1023 at the proximal end of element 820. This can have utilitarian value with respect to guiding a terminal of a syringe to the passage through element 820. Thus, embodiments can include a guide configured to guide a tip of a termination of a syringe located outside the inner ear to the septum and/or a passage of the tissue interface portion leading to the septum. And the guide need not be on the passive component. FIG. 24 presents an exemplary implant 2451, that includes self-healing septum 2444 (which is the passive component). A funnel 2588 is attached to the body 810, and thus the guide is not part of the passive component.

FIG. 11 presents another exemplary embodiment of a relatively large septum 1130. Here, the septum 1130 is located in the tubular body 1011. The tubular body 1011 has in the passageway therethrough, a spring ring 1122 located therein, which prevents distal movement of the septum 1130. In an alternate embodiment, spring ring 1120 could instead be a ring screwed into the inside of the tube 1011. In this regard, the inside of tube 1011 could be threaded. In this regard, in some embodiments, the septum 1130 is threaded into the tube 1011. The septum 1130 may or may not have threads—the threaded coupling can be established by the flexible nature of the septum 1130. As seen, the proximal portion of the septum 1130 is proud of the proximal portion of the body 1011. This can have utilitarian value with respect to enabling removal and/or replacement of the septum 1130, such as can be the case in the event of wear and/or age of the septum 1130. It is also noted that in an exemplary embodiment, element 1130 could instead be a plug. The plug can be removed to enable access into the inner ear cavity 199. The plug can be a rigid plug or operate in the form of a cork. Further, the proximal end of the plug 1130 or septum for that matter could also have a wider portion otherwise could be such that the overall structure is T-shaped, to enable a larger area to be gripped in the event of removal—a “target” could be located in the center of the septum to provide an indication for where the termination of the syringe should be located so as to reduce the likelihood that the termination would be inserted into the septum at a location where it might hit the wall structure of the body 1011. In an exemplary embodiment, there is a device that interfaces with the port that has a syringe termination, wherein the device self-aligns the termination when engaged with the port, thereby reducing or otherwise eliminating the risk that the termination will hit the wall of the port component. Moreover, the device could be an injector that has no exposed needle until locked into the port, then needle is advanced, always at the correct angle, through the septum.

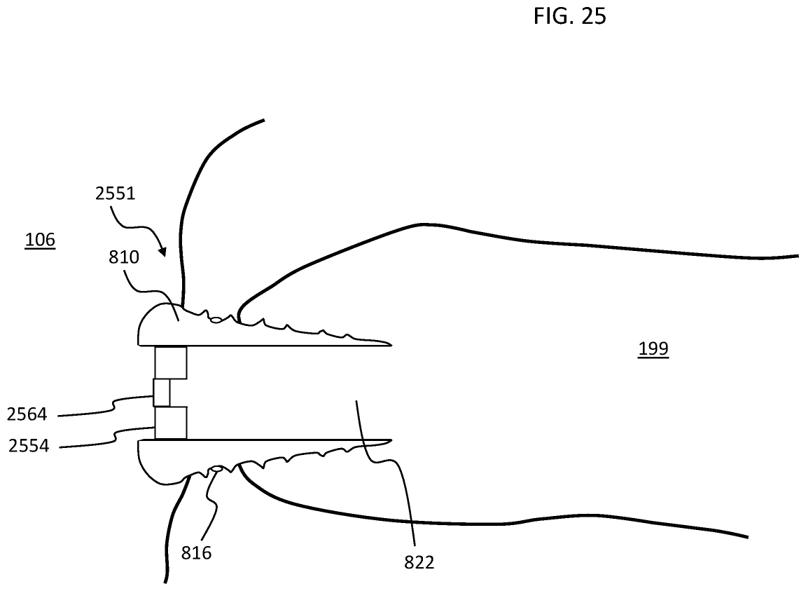

FIG. 25 presents an exemplary implant 2551 that includes a different type of septum arrangement. Here, the septum includes an outer septum portion 2544 that is relatively thick-walled and an inner septum portion 2564 that is relatively thin-walled as shown. In an exemplary embodiment, both portions of the septum can be pierced, but with some semi-careful alignment of the termination, the thin-walled septum can be repeatedly or more frequently pierced relative to the thick-walled.

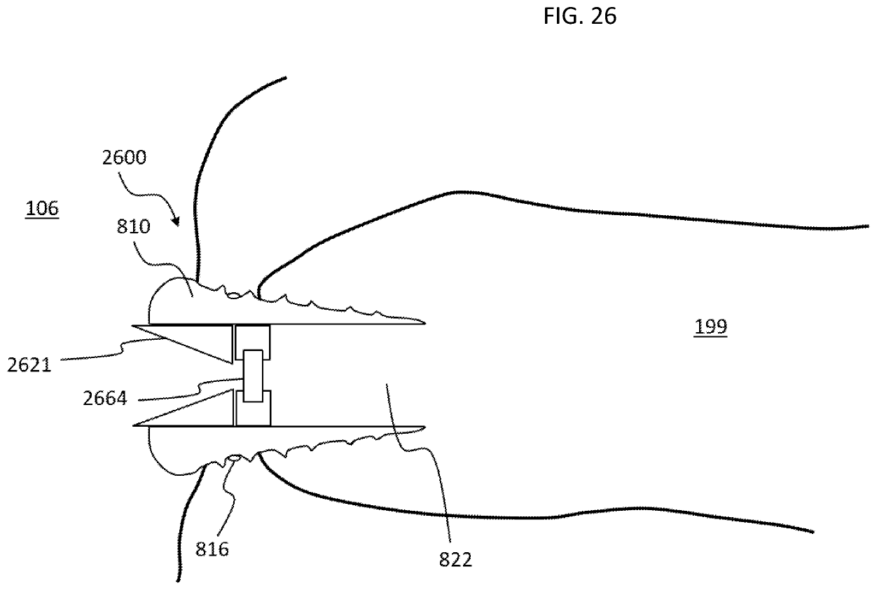

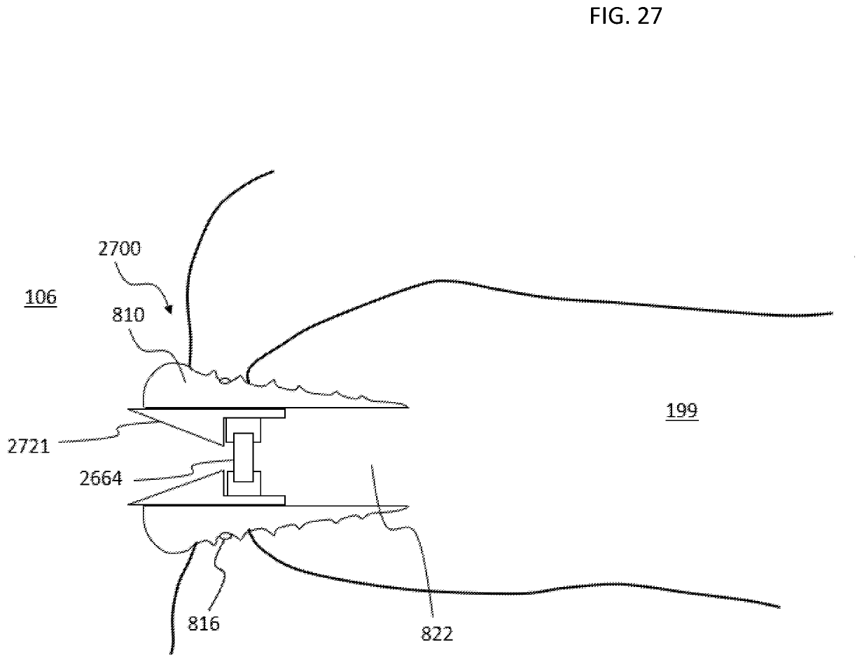

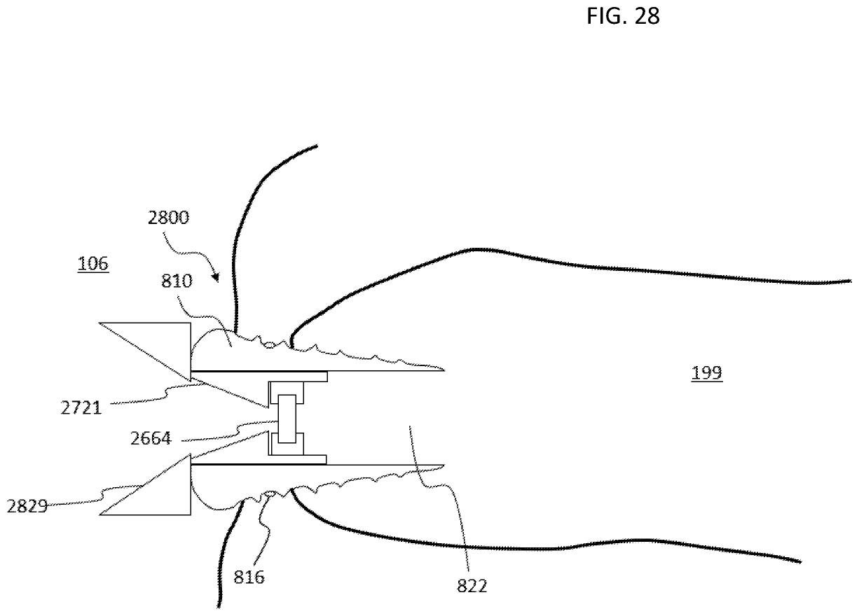

As shown in FIG. 25, the thin-walled septum portion 2564 is located proud of the thick-walled septum portion 2554. In an alternate embodiment, as seen in FIG. 26, the thin-walled septum portion can be recessed relative to the forward facing surface of the thick-walled septum portion. Indeed, as shown in FIG. 26, the thin-walled septum portion 2664 is also located in a manner where the thick-walled septum portion extends over the top and bottom of a portion of the thin-walled septum portion 2664. Also shown in FIG. 26, with respect to the implant 2700, there can be seen a third component 2621, that is in the form of a rotationally symmetric body (again, for purposes of illustration, the back lines are not shown in many instances—in real life, there would be a vertical line at the left most side any vertical line at the right most side of the body 2621) that has a cone shaped interior as can be seen. This can have utilitarian value with respect to guiding the tip of the termination to the septum in general, and the thin-walled septum portion 2664 in particular. And in this embodiment, it can be seen that the guide body 2621 is a separate component entirely from the septum portion, where removal of the guide body 2621 would not move the septum. FIG. 27 presents an exemplary embodiment of an implant 2700 where the guide body and the septum are part of a single component 2721 (the septum can be locked into the guide body with a spring lock or glue or a second body that is screwed to the distal bore). In an exemplary embodiment, the body 2721 can be screwed into body 810 or interference fitted, etc. Also, while the conical shape for the termination guide is shown as being limited to the diameter of the bore 822, in an alternate embodiment, the termination guide can extend further outward such as shown in FIG. 28. Here, there is an exemplary implant 2800, where the component 2721 of FIG. 27 further includes an additional portion 2829, which can be a component that is machined from the stock that was utilized to create the body 2621 or can be an out on part that is interference fitted or glued or otherwise sintered or welded to the body 2621. Any device, system and/or method that can enable a termination guide can be utilized in at least some exemplary embodiments.

And in an exemplary embodiment, the targets can be applied to the outer surface of the septum so that the healthcare professional can better aim the tip of the termination. For example, a bull's-eye target such as a series of concentric circles can be utilized. Alternatively, and/or in addition to this, crosshairs can be utilized. In some embodiments, there can be a hard visible material below the septum and/or on a far-side portion of the septum (relative to the middle ear). This can stop the termination from over insertion and/or enable visualization of the septum/target. Different color targets can be used, such as ruby colored targets.

In an exemplary embodiment, the septum can be located within a rigid ring, such as a ring made out of titanium or a titanium alloy or some other biocompatible metal. In this regard, referring back to FIG. 26, element 2664 can be the septum, and the supporting element can be the ring. Also, referring to where the septum is supported by tissue interface device, the body 810 can be a rigid ring and/or a grommet (more on this below) and/or a hollow cylinder. A polymer plug can be utilized to plug the passage therethrough, which polymer plug can be in accordance with any of the teachings detailed herein. And in this regard, while the self-healing septum can be utilized, in addition to this, the cap or plug can be utilized to seal at more proximal locations from the septum if a belt and suspenders approach where redundancy approaches deemed utilitarian.

And it is also noted that while the aforementioned septums have been described in terms of a device that enables a termination to be passed therethrough, it is also noted that in other embodiments, other devices can be passed therethrough, such as, for example, the electrode array of the cochlear implants, or other therapeutic substances, such as a solid therapeutic substance, or a sensor/a boom that supports a sensor at the distal end thereof, etc. (This may or may not be a self-healing septum, depending on the size of the portion extending therethrough.) Indeed, in an exemplary embodiment, a module that includes a body that has a sealing feature at the proximal end thereof, that seals at the opening of the body 810 when fully inserted into the body 810, can have a boom that is attached to or otherwise extends from the body, which boom contains a sensor, and the boom extends through the opening in the septum, so that the sensor can come into contact with the perilymph of the middle ear. The septum will seal around the boom, and then the sealing features of the body at the proximal end thereof will also seal the passageway.

Embodiments of the septums detailed herein, such as the self-healing septums, or any the other port device for that matter, are configured to avoid leakage of fluid from within the cochlea or otherwise within the inner ear to the middle ear or otherwise outside the inner ear, or at least avoid substantial leakage that would have a noticeable deleterious effect and/or an annoyance effect. By way of example only and not by way of limitation, a leakage rate can be limited to 0.1 to (no more than) 10 microliters or any value or range of values therebetween in 0.01 microliter increments. These can be absolute values, or values that occur after a period of time lasting 1, 2, 3, 4, 5, 6, 7, 8, 9, or 10 days, or weeks, or any value or range of values therebetween in 1 hour increments.

Indeed, in this regard, embodiments of the plugs and/or other devices that “fill” the body that interfaces with the tissue of the inner ear detailed herein, or the body itself for that matter, are configured to avoid leakage of fluid from within the cochlea or otherwise within the inner ear to the middle ear or otherwise outside the inner ear, or at least avoid substantial leakage that would have a noticeable deleterious effect and/or an annoyance effect. Leakage can be limited to the aforementioned values.

In an exemplary embodiment, plugs/bodies located in the tissue interfacing body can be made of a silicone or a polymer and/or a low durometer polymer. The septum can be configured for utilization with a non-coring needle/termination, and thus the teachings detailed herein can be utilized with such and include methods of utilizing such.

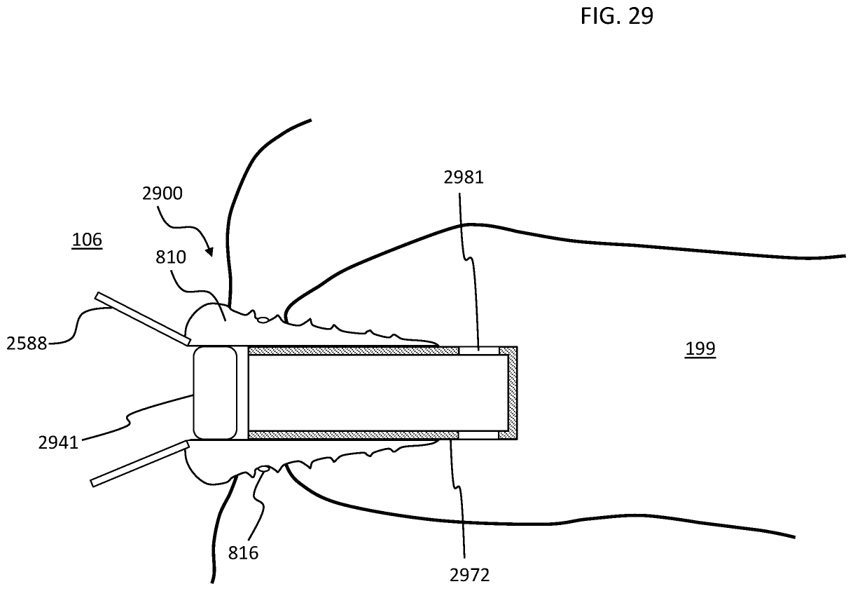

In an exemplary embodiment, the port implant can include a termination over-insertion prevention device configured to prevent over insertion of a termination of a syringe extended through a septum into the inner ear. In this regard, FIG. 29 presents an exemplary implant 2900 including the tissue interfacing component 810 in which is located a self-healing septum 2441. Distal of the septum 2441 and also located in the tissue interfacing component 810 is a closes bottom cylinder 2972. Cylinder 2972 is open with respect to the side facing the septum 2941. The closed bottom creates a stop for the tip of the termination. As can be seen, there are openings 2981 on the sidewalls of the cylinder 2972 at the distal portions thereof. In an exemplary embodiment, this permits the fluid delivered by the termination of a syringe to exit the implanted 2951 and enter the duct 199. But because the termination will be relatively rigid, the termination will not be able to go through the openings 2981 owing to the limited angle that the overall structure will permit the termination to achieve when the termination is located in the cylinder 2972. And while the proximal portions of the cylinder 2972 are shown as relatively blunt end walls, in other embodiments, the ends of the end walls can be chamfered or otherwise angled so that even a very uncentered termination will be guided into the hollow portion of the cylinder 2972.

Embodiments can include a system that includes an inner ear barrier tissue interface apparatus (e.g., element 810) through which a passage extends, wherein the inner ear barrier tissue interface apparatus is configured to permanently fix to an opening in a barrier between a middle ear and an inner ear of a human such that the passage extends from the middle ear to the inner ear. In this exemplary embodiment, the system further includes a first functional component at least partially located in the passage. In this exemplary embodiment, at least one of (i) the first functional component is a passive first functional component detachably attached directly or indirectly to the tissue interface apparatus and removal of the first component provides physical access from the middle ear into the inner ear through the passage, wherein the first functional component at least partially seals the passage in addition to providing a passive feature; (ii) the tissue interface apparatus includes a second passage that is unsealably sealed, the second passage providing physical access from the middle ear into the inner ear through the second passage bypassing the first functional component or the first functional component is a passive first functional component detachably attached directly or indirectly to the tissue interface apparatus and removal of the first component provides physical access from the middle ear into the inner ear through the passage, wherein the system further includes a removable seal apparatus (a plug and/or a cap, for example), configured to unsealably seal the passage, the removable seal apparatus being separate from the first functional component; or (iii) the first functional component has a passage that is unsealably sealed, the passage of the first component providing physical access from a middle ear facing side to an inner ear facing side when unsealed with the first functional component in the passage of the tissue interface apparatus.

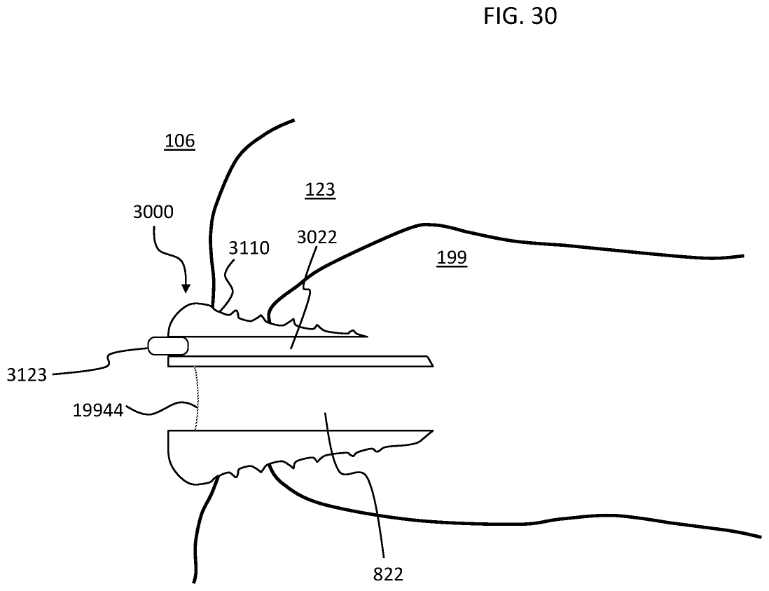

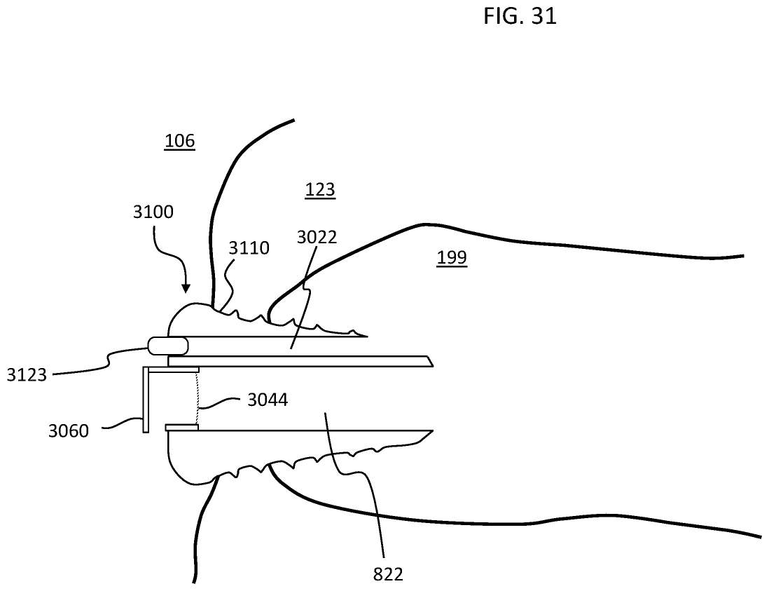

With respect to “i,” the various embodiments above bear this out. With respect to “ii,” FIG. 30 presents an exemplary implantable port prosthesis 3000 that includes a tissue interface component 3110 that has the traditional passage 822 therethrough as can be seen, along with a second passage 3022. In this exemplary embodiment, an artificial round window 19944 is permanently fixed in the passage 822. Conversely, FIG. 31 presents an exemplary port prostheses 3100 where the first functional component is an assembly that includes an artificial round window 3044 supported by a removably closed bottom cylinder 3060 located in passage 822, the cylinder 3060 removably in a manner concomitant on with the other components detailed herein that are located in the passage 822. Thus, as can be seen with respect to both these embodiments, with seal (or septum 3123) located in the second passage 3022, the inner ear barrier tissue interface includes a second passage that is unsealably sealed, the second passage providing physical access from the middle ear into the inner ear through the second passage bypassing the first functional component. In this regard, in an exemplary embodiment, the termination can be inserted into passage 3022 from the middle ear side when the plug 3123 is removed (or by piercing element 3123 if such is a self-healing septum for example) and a therapeutic substance can be transported into the duct 199. Alternatively, a borescope or some other device or probe could be inserted through the passage, or some active component for that matter. A sensor could be located in the second passage 3022. With respect to a scenario where the termination is utilized to access the duct 199 via passageway 3022, returning back to FIG. 31, the closed bottom of the cylinder 3060 has utilitarian value with respect to providing a shroud that protects the artificial window 3044 from the sharp tip of the termination. As can be seen, there is an opening in the side of the cylinder 3060 to ensure that pressure changes on the middle ear side of the artificial round window can be sufficiently uninhibited that the functionality of the artificial round window 344 is unimpeded.

In some embodiments, the device is configured, when the plug or cap, etc., is removed from the device, so that the passageway is open when the component is releasably attached to the tissue interface portion and/or a portion of the device supported by the tissue interface portion. In some embodiments, the component is configured, when the plug/cap etc., is removed from the device to unsealably seal a local portion of the passageway when the component is releasably attached to the tissue interface portion and/or a portion of the device supported by the tissue interface portion. There can also be a passageway thought the component, which passageway can also be sealed by another plug or cap (there can be two plugs or caps, or can be only that sealing the passage of the component).

With respect to “iii,” the first functional component has a passage that is unsealably sealed, the passage of the first component providing physical access from a middle ear facing side to an inner ear facing side when unsealed with the first functional component in the passage of the inner ear barrier tissue interface apparatus, this can correspond to the implant 1800 of FIG. 19 above, where septum 2222 can be punctured to unseal the established seal. This can also be device 2301 (but not implant 2451, where there is no passage in the passive component). Note also that this feature (“iii”) does not require that the first functional component is removable, at least not in its entirety (a cap or seal or plug of the first functional component could be removable so as to establish access to the passage of the first functional component that provides physical access from the middle ear facing side to the inner ear facing side). In an exemplary embodiment, the port prostheses can have one or more or all of elements “i,” “ii,” or “iii,” providing that the art enable such. Consistent with the teachings above, the first component can be an artificial round window, and the inner ear barrier tissue interface apparatus can be located at a natural round window location of a human and the artificial round window replaces a natural round window of the person. Also consistent with the teachings above, the first component can be a passive drug-release device, and/or the first component can be a self-healing septum.

In an exemplary embodiment, the first component is a therapeutic substance containing reservoir that is compressible due to pressure increase in the middle ear. In an exemplary embodiment, there is a method of increasing the middle ear pressure which compresses a drug containing reservoir which is in fluid communication with the inner ear and exposed to the middle ear (and there is a device that enables such). Increased middle ear pressure can be achieved by depositing a gassing agent in the middle ear (this agent might block the Eustachian tube to allow for pressure to build up, a catheter to pump air into the middle ear coming through the Eustachian tube, or by swallowing).

An exemplary embodiment can be seen in FIG. 31. Here, reservoir 1340 is compressible or otherwise flexible. If the pressure within the middle ear cavity 106 is raised above a certain level, this will apply pressure on to the outer surface of the reservoir 1340, which will in turn increase the internal pressure, and thus drive the therapeutic substance from the reservoir 1340 into the conduit 1350. If the pressure is high enough to overcome the ball check valve 3260 that establishes only one way flow in the conduit 1350, the therapeutic substance will continue to flow into passageway 819 and then into the duct of the cochlea 199.

As noted above, in an exemplary embodiment, there are methods of increasing the pressure within the middle ear cavity and devices for such, such as, for example, a device that can intermittently or controllably block the Eustachian tube, which could be a plug that is sized and dimensioned to be inserted into the tube and removed from the tube. Alternatively, a gas generator could be located in the middle ear. Alternatively, a syringe could be inserted through the tympanic membrane a limited distance, which syringe is filled with air or an inert gas or an inert liquid, and that gas could be inserted in the middle ear to raise the pressure therein.

Thus, in an exemplary embodiment, there is the first component that is a therapeutic substance containing reservoir that is compressible due to atmospheric pressure increase in the middle ear, wherein compression of the first component due to the atmospheric pressure increase in the middle ear drives a therapeutic substance contained in the reservoir into the inner ear. This is distinguished from a reservoir that is compressible by, for example, the closure of a forceps or a tweezers for example. To be clear, the reservoir can be also compressed by such, but here, it is the atmospheric pressure increase that results in the compression and subsequent transportation of the therapeutic substance into the cochlea. Along these lines, in an exemplary embodiment, the reservoir is configured to be compressed so as to transport the therapeutic substance into the cochlea upon an increase of pressure within the middle ear cavity of a value at least and/or equal to and/or no greater than 5, 10, 15, 20, 25, 30, 35, 40, 45, 50, 55, 60, 65, 70 percent, or any value or range of values therebetween in 1% increments increase from an average pressure and/or a baseline pressure and/or from 14.9 PSI.

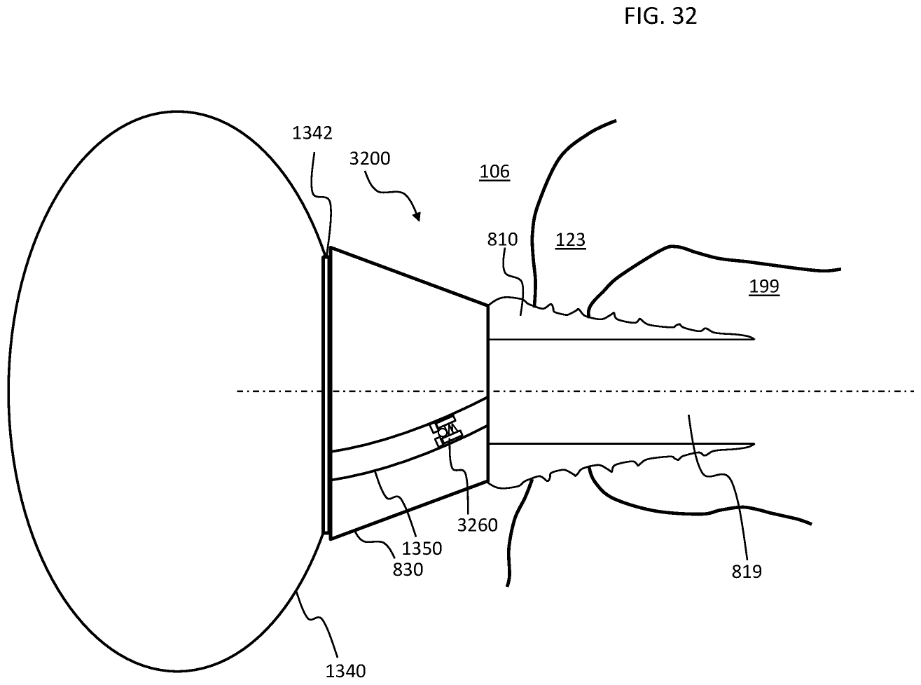

And in an alternate embodiment, the reservoir is configured such that a change in atmospheric pressure, such as those detailed above, will not result in therapeutic substance delivered to the cochlea-here reservoir would have to be compressed by some form of tweezers or forceps (or a blunt body driven forward (left to right in FIG. 32)—such as a blunt object that is pushed by a force extending through an incision/grommet in the tympanic membrane) for example to result in the therapeutic substance delivery into the cochlea, and therefore, in some embodiments, the first component is a therapeutic substance containing reservoir that is compressible due to a compression greater than an atmospheric pressure increase in the middle ear of 50, 60, 70, 80, 90, 100, 125, 150, 175, 200, 300 or 400 percent over the baseline or any value or range of values therebetween in 1% increments, wherein compression of the first component due to the mechanical compression drives the therapeutic substance contained in the reservoir into the inner ear.

And in some embodiments, the increase in pressure in the middle ear (or the actions of the tweezers) can be pulsatile or otherwise segmented in time (followed by a decrease in pressure) so as to achieve repeated delivery doses separated in time. The increases in pressure can follow the other delivery regimes detailed above that deliver the therapeutic substance over a period of time in discrete increments.

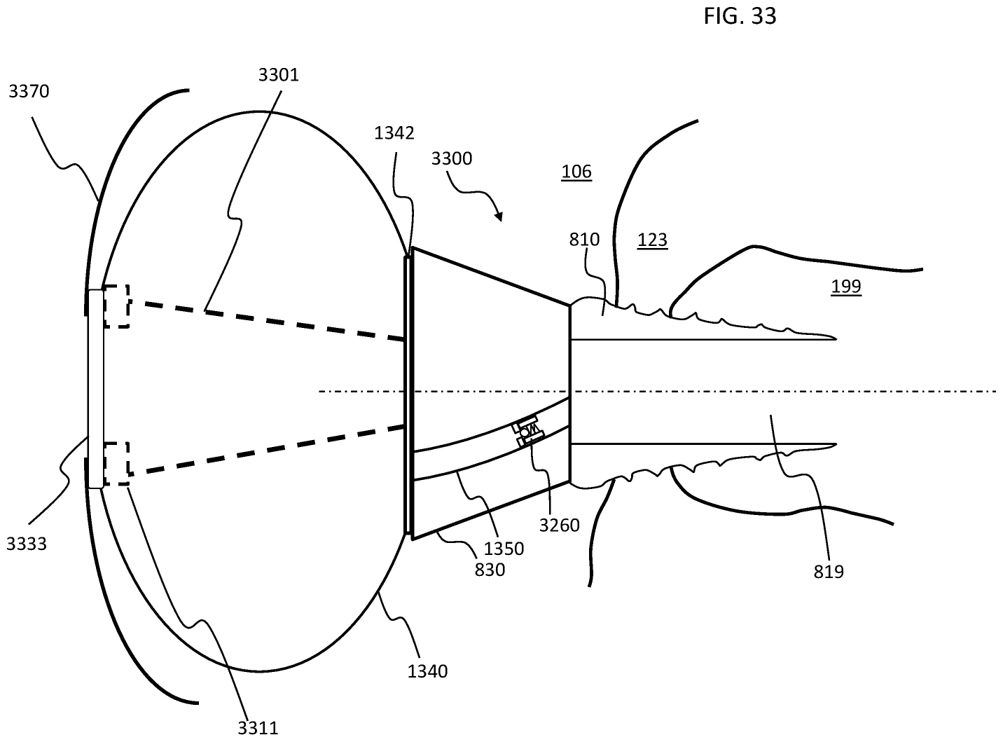

In an exemplary embodiment, the first component is a therapeutic substance containing reservoir that has a self-healing septum facing the middle ear when attached to the inner ear barrier tissue interface and is in fluid communication with the passage. This can be seen in FIG. 33, which depicts an implant 3300, where septum 3333 is located on the proximal portion of the device. In this embodiment, a support structure 3301 is located in the reservoir 1340 (shown in dashed lines because support structure 3301 is located inside the reservoir and thus not visible from the outside unless the reservoir is a transparent reservoir-support structure 3301 comprises two tubular beams that extends to a circular ring 3311 that supports the septum 3333. In this exemplary embodiment, when the termination is pressed through the septum 3333, the ring 3311 resists the right workforce owing to the fact that the ring 3311 is supported by the beams 3301, and thus the reservoir 1340 is not compressed or otherwise collapsed.

Also as seen in FIG. 33 is a middle ear armor device 3370. In this exemplary embodiment, a metal such as titanium or stainless steel is arrayed over a portion of the septum 3333 is shown, and arcs away from the septum around and over the reservoir 3470. In an exemplary embodiment, this can have utilitarian value with respect to preventing contact between the sharp tip of the termination and the potentially delicate or otherwise flexible material of the reservoir 1340 when a termination is utilized to pierce the septum 3333 to provide therapeutic substance into the reservoir with respect to the execution of a refilling action. In an exemplary embodiment, the tip of the termination would slide up/down/left/right along the accurate surface of the armor 3370 and thus to the side of the reservoir 1340. The armor could be glued to the outer portions of the septum, or can be supported by the support structure 3310 such as an embodiment where the ring 3310 extends from inside the reservoir two outside the reservoir (and the material of the reservoir is connected to the ring on the sides of the ring for example).

In an exemplary embodiment, the first component is a therapeutic substance containing reservoir that has a self-healing septum facing the middle ear when attached to the inner ear barrier tissue interface and the system is configured so that flow from the reservoir into the inner ear through the barrier is restricted. By way of example, in one implementation the drug reservoir is a closed space, in the port, made from a septum facing the middle ear space to allow filling with a needle, to an inner wall that is a reservoir, the perilymph facing side covered by a flow restricting membrane/microbiological filter that allows small molecules to pass but does not allow pathogens to pass. Therapeutic substance release can be achieved by any of the teachings detailed herein and/or variations thereof.

And while the embodiments above have been presented in terms of a reservoir that can be refilled, in an alternate embodiment and/or in addition to this, the therapeutic substance reservoir can be implanted in a prefilled manner, and can be replaced when empty or otherwise replaced if let us say another therapeutic substance is deemed to have more utilitarian value than that which is contained in the reservoir. Thus, in an exemplary embodiment, the first component can be a prefilled reservoir without refill capability or otherwise without the ability to refill the reservoir, at least with respect to scenarios when the reservoir is attached to the tissue interface component or otherwise is implanted in the recipient. In an exemplary embodiment, the reservoirs are sized and dimensioned or otherwise configured so that a total load of the therapeutic substance, in a scenario where all of the therapeutic substance was released in a relatively short time, such as all the once, in the event of an accident, or otherwise in the event of a failure mode, would not result in a toxic level and/or an above toxic level of therapeutic substance being released into the person. Alternatively, and/or in addition this, multiple reservoirs could be utilized that have these features individually, where the likelihood of a series of failures were therapeutic substances into or more reservoir is being released at the same time otherwise in close proximity is unlikely as a matter of statistics.

Embodiments of the plugs and/or other devices that “fill” the body that interfaces with the tissue of the inner ear detailed herein, or the body itself for that matter, are configured to avoid leakage of fluid from within the cochlea or otherwise within the inner ear to the middle ear or otherwise outside the inner ear, or at least avoid substantial leakage that would have a noticeable deleterious effect and/or an annoyance effect. By way of example only and not by way of limitation, a leakage rate can be limited to 0.1 to 10 microliters or any value or range of values therebetween in 0.01 microliter increments.

In an exemplary embodiment, plugs/bodies located in the tissue interfacing body can be made of a silicone or a polymer and/or a low durometer polymer. The septum can be configured for utilization with a non-coring needle/termination, and thus the teachings detailed herein can be utilized with such and include methods of utilizing such.

Thus, in some embodiments, the inner ear port device is made of a single unit, while in other embodiments, the inner ear port device is made of multiple units. The inner ear port device can be configured to be secured into the labyrinth (cochlea, semi-circular canals, and/or otolith, depending on the embodiment), and can be utilized to provide direct access to inner ear fluid and/or tissue (perilymph, endolymph, etc.). In some exemplary embodiments, the enablement of the direct access to inner ear fluid can enable measurements of biomarkers in inner ear fluid, can enable delivery of drugs and/or other substances, including implants into inner ear fluid, and/or can enable sampling of inner ear fluid to allow for analysis inside the port and/or outside the body. One implementation of the inner ear port, as seen above, includes two units, where one is configured to be secured into and/or to bone or tissue and the other is configured to be attached to and/or inserted into the unit secured into bone or tissue.

Thus, consistent with the teachings detailed above, in at least some exemplary embodiments, there is an inner ear device, comprising a tissue interface portion configured to attach to tissue of and/or proximate an inner ear of a human and provide a passage from outside the inner ear to inside the inner ear. This can correspond to, for example, body 810 alone or the combination of the components detailed above with respect to some of the embodiments. In an exemplary embodiment, the device further includes a container releasably attached to the tissue interface portion and/or a portion supported by the tissue interface portion (e.g., body 820 if body 820 is used in combination with body 810, or head 888, if head is used in combination with body 810—of course, if head 888 is part of body 810, then that is the “releasable attached to the tissue interface portion). In this exemplary embodiment, the container is configured to contain a therapeutic substance, and in some embodiments, the container contains the therapeutic substance.

Here, the inner ear device is configured to only operate in a massive manner or to be controlled by physical manipulation.

In an exemplary embodiment, the inner ear device has no stimulative capabilities. In an exemplary embodiment, the inner ear device has no componentry configured to electrically stimulate tissue to evoke a sensory response. In an exemplary embodiment, the inner ear device has no componentry configured to mechanically stimulate tissue and/or fluid to evoke a sensory response.

In some embodiments, the inner ear device is configured to release the therapeutic substance contained in the container through passive transportation. Thus, here, it can be seen that the implant can include a drug or other therapeutic substance reservoir that can be replaced (and/or refilled-more on this in a moment). The drug or other therapeutic substance can be released into the inner ear though passive mechanisms, such as by way of example only and not by way of limitation, diffusion through a semi-permeable membrane. Alternatively, and/or in addition to this, the drug or other therapeutic substance can be released via pressure build up in the middle ear and/or directly in the reservoir using the mechanical and/or chemical reaction, such as via the use of phase changing materials, electrolysis, etc. The drug or other therapeutic substance influx can be regulated by way of example only and not by way of limitation, utilizing valves and/or the pressure alone and/or in combination with inlet holes/pores, stretchable membranes, etc.

As noted above, in some exemplary embodiments, the drug reservoir is releasable from the tissue interface component (and/or a component between the tissue interface component, such as the head 888). In other embodiments, the drug reservoir is permanently attached. In an exemplary embodiment where the drug reservoir is permanently attached, the drug reservoir can be refilled or otherwise resupplied. In an exemplary embodiment, this can entail conveying a needle/termination of a syringe into the middle ear, and piercing a septum or the like of the reservoir, which septum can be self-healing, and then refilling the reservoir. In another exemplary embodiment, by way of example only and not by way of limitation, a refilling port can be located on the reservoir or in fluid communication there with, and a refueling tube or some other device can be attached to the port, and the therapeutic substance can be delivered therethrough.

It is noted that while the above embodiment associated with refilling or otherwise resupplying the container was described with a container that is fixed and otherwise not releasably attached to the tissue interface component or whatever component is an issue, in other embodiments, one or more of the above features associated with refilling can be applicable to the container that is releasably attached.

It is further noted that the phrase “releasably attached” refers to a structure that enables the container to be readily detached in a normal and expected manner so as to permit resupplied. This is as distinguished from, for example, the mere ability to disassemble various components. That is, even if, for example, the container could be saved for example, cut from the tissue interface, such would not correspond to releasably attached.

And further to the embodiment of FIG. 13, in an exemplary embodiment, the inner ear device can include a fluid valve between fluid and the inner ear and an outside of the inner ear. The valve is shown in FIG. 13 as valve 1360. In at least some exemplary embodiments, the implant is configured so that the valve can be adjusted to control an amount of therapeutic substance to be released into the inner ear from the container. The valve can be controlled in any one or more of the aforementioned ways to accomplish any one or more of the aforementioned method actions. The valve can be utilized in conjunction with other control functionalities, such as, for example, regulating the pressure within the reservoir 1340 and/or the pressure behind the valve 1360.

In an exemplary embodiment, the valve or other therapeutic substance delivery regulation means is configured to and/or controlled to prevent deleterious and/or annoying pressure fluctuations within the inner ear. In an exemplary embodiment, the valve or other therapeutic substance delivery regulation means is configured to and/or controlled to maintain a level of pressure within a certain boundary, limit any pressure fluctuations to within a certain range. In an exemplary embodiment, the actions detailed herein are executed and the devices and systems enable the execution of a therapeutic substance delivery or any other actions detailed herein in some embodiments, into the inner ear that maintains the pressure within the inner ear within a 5, 6, 7, 8, 9, 10, 11, 12, 13, 14, or 15% variance from a mean, median, and/or mode and/or from a baseline pressure (the pressure just before the start of the action). In an exemplary embodiment, the pressure regulation is passive, such as where, for example, a spring loaded valve is arranged, which valve opens upon the attainment of a certain pressure.

Consistent with the teachings detailed above, in some exemplary embodiments, the tissue interface is located in bone establishing a barrier between the middle ear and the inner ear. In some exemplary embodiments, the tissue interface has been implanted in the bone for at least and/or equal to 3, 4, 5, 6, 7, 8, 9, 10, 11 months, or 1, 2, 3, 4, 5, 6, 7, 8, 9, 10, 11, 12, 13, 14, 15, 16, 17, 18, 19, or 20 or more years, or any value or range of values therebetween in one week increments.

In an exemplary embodiment, the passive component was implanted for or for at least 1, 2, 3, 4, 5, 6, 7, 8, 9, 10, 11, 12, 13, 14, 15, 16, 17, 18, 19, or 20 or more, or any value or range of values in 0.1 increments days or weeks or months or years less than the tissue interfacing portion.

Some embodiments include the action of utilizing the inner ear port device as a cochleostomy without a sheath for insertion of a cochlear implant electrode array.

Some embodiments include an inner ear device that is a device that is dedicated to the functionality of establishing long-term biocompatible ready access to the inner ear from the middle ear. This as distinguished from, for example, a cochlear implant, where a portion of the implant extends from the middle ear into the inner ear. Such a device is configured to provide sensory stimulation. In some exemplary embodiments, the inner ear device has no componentry configured to electrically and/or mechanically stimulate tissue. In an exemplary embodiment, the inner ear device has no componentry configured to evoke a sensory response of the human. In an exemplary embodiment, the inner ear device has no componentry configured to electrically and/or mechanically stimulate tissue to evoke a sensory response of the human. This is distinguished from, for example, the potential that one or more of the electrical devices detailed herein may apply current somehow to tissue of the recipient. Further, in some embodiments, even if there is stimulation to tissue, providing that does not evoke a sensory response of the human, such would still be within the scope of some embodiments. To be clear, in some embodiments, there is no arrangements of the inner ear device that is stimulative. In some embodiments, the purpose of the implant is to provide the long-term ability to access the inner ear from the middle ear.

Thus, in view of the above, it can be seen that in some embodiments, there is a device, comprising a tissue interface portion configured for securement to tissue of and/or proximate an inner ear of a human and provide a long term passage from outside the inner ear to inside the inner ear, and a therapeutic substance at least indirectly releasably attached to the tissue interface portion and/or a portion of the device supported by the tissue interface portion, wherein the device is configured to passively provide a therapeutic substance to an inner ear, and the therapeutic substance is configured to be located entirely within a middle ear cavity and/or the inner ear of a human.

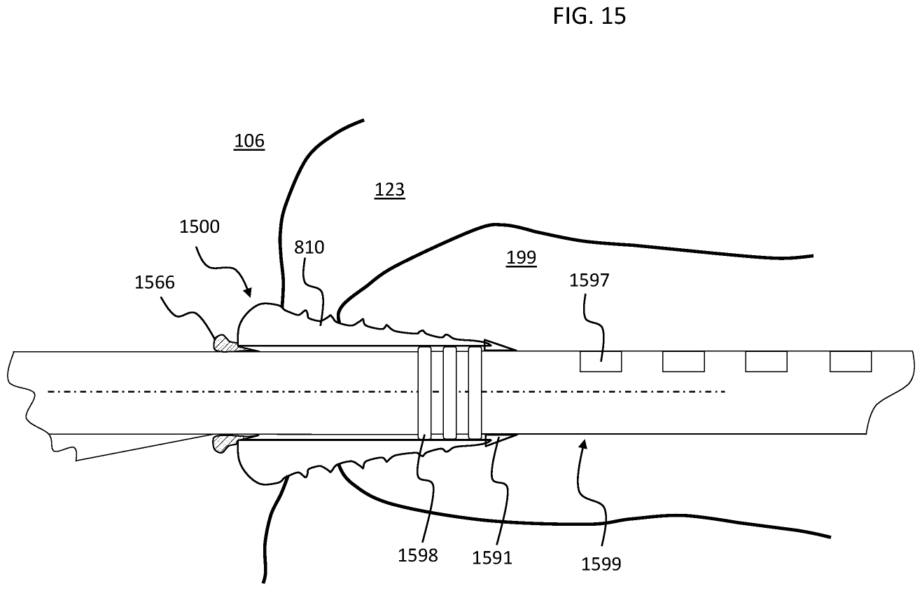

But note that some embodiments can utilize the inner ear device to access the inner ear for the purposes of inserting a device that is configured to stimulate the cochlea. By way of example only and not by way of limitation, FIG. 15 presents an exemplary embodiment of an inner ear port 1500. Here, the port 1500 includes the body 810, which can correspond to the above embodiment. In this arrangement, a cochlear implant electrode array 1599 extends through the inner ear port device 1500. Note that the cochlear implant electrode array 1599 is not part of the inner ear port device 1500. Instead, it is utilized in conjunction therewith. In this regard, in an exemplary embodiment, a previously passive component could be removed from the throughbore of the body 810 and the cochlear implant electrode array can replace that passive component. To be clear, what is shown in FIG. 15 is not a passive inner ear port implant. What is shown in FIG. 15 is a system that includes an inner ear port device 1500 and a cochlear implant electrode array (in fact, because the cochlear implant electrode array is attached to the receiver stimulator of the cochlear implant, what is shown in FIG. 15 is also a system that includes the inner ear port device 1500 and an implantable portion of a cochlear implant). In this exemplary embodiment, the cochlear implant electrode array 1599 includes a plurality of stimulating electrodes 1597, some of which are shown (the full extent of the array is not shown for purposes of schematic economy). Also shown are ribs 1598. These ribs abut the inner surface of the passageway through body 810, and can establish a fluid tight seal. In an exemplary embodiment, the electrode array 1599 is specially designed to work with the inner ear port device 1500. In an exemplary embodiment, a “wedge seal” 1565 is part of the electrode array 1599. As the electrode array is inserted into the cochlea through the passageway in the body 810 the pertinent distance, the wedge seal 1566 enters the passageway through the body 810, and wedges itself therein, thus establishing a second seal in addition to the ribs 1598. The above said, in an exemplary embodiment, a separate seal instead and/or in addition to this can be placed at the interface. In an exemplary embodiment, a form-in-place seal can be applied after the electrode array 1599 is inserted into the cochlea. That said, the locations of the electrode array that can be estimated to be proximate the inner surface of the body can be covered with the form-in-place seal material such that when the electrode array is inserted through the passage, the sealing material which has been carried into the passage by the electrode array 1599 contacts the inner surface of the body 810, and thus establishes a seal after a modicum of curing.

In an exemplary embodiment, the electrode array 1599 has components that prevents further insertion into the cochlea and/or prevent movement of the electrode array 1599 in the opposite direction, or otherwise frustrate such movements. By way of example only and not by way of limitation, as can be seen in FIG. 15, there is a wedge seal 1591 that extends about the outer periphery of the intracochlear portion of the electrode array 1599. In an exemplary embodiment, after the election array is inserted a sufficient distance, and the compressible material of the wedge seal 1591 (the compressible material could be silicone for example) clears the distal and of the passageway of the body 810, thus permitting the compressed wedge seal 1591 to “spring” outward again, thus pass the inner diameter of the inner passage, a slight pullback on the electrode array can jam the relatively sharp edges (in this exemplary embodiment) of the body 810 into the wedge seal 1591 is shown, thus creating an additional seal. But even without this additional sealing, this exemplary embodiment permits the electrode array 1599 to be held relatively stationary or otherwise prevents or otherwise frustrates rearward movement of the electrode array out of the cochlea. In an exemplary embodiment, seal 1566 is added after the election array is so positioned, and the seal 1566 can bond or otherwise correct the body of the extra cochlear portion of the electrode array, thus preventing the electrode array from moving forward or otherwise frustrating forward movement, and, in this embodiment, providing for additional sealing. Note also that in an exemplary embodiment, the surface of the passageway through the body 810 could be roughened so as to increase the friction forces against the ribs 1598. And in some embodiments, instead of the ribs and/or in addition to the ribs, the overall outer profile of the electrode array could be larger than the inner diameter of the passageway the body 810, so as to provide a friction fit and/or an interference fit to frustrate movement of the electrode array in the various directions. Also, such a fitting regimes can also create a seal, at least in embodiments where, for example, the electrode array is made of silicone at the interfacing portion.

An exemplary embodiment includes a minimally-invasive implantation method for a cochlear implant electrode array, which electrode array results in the ability to provide electrical stimulation to ganglion cells responsible for sensing higher frequency sounds. This method also includes the utilization of components, such as, for example, a grommet that is adapted for insertion into a cochleostomy formed in bony tissue adjacent the round window by way of example. The grommet can have an actual passageway through the center thereof. The array can be configured to be inserted through the actual passageway of the grommet, and methods include doing so. The electrode array and/or the grommet has a cross-sectional size that enables the array to snugly engage and otherwise fill the axial passageway when the electrode array is inserted to the prescribed depth. This snug fit prevents fluids, such as perilymph, from not passing through the axial passageway when the electrode array is inserted in the axial passageway. In some exemplary embodiments, there is a grommet, which can have any one or more of the features detailed above associated with the body 810, comprising a conical shape member having threads on an outer surface thereof, and a slot on a backside thereof. This lot can be configured to receive a flat head of a screwdriver or the like to enable the grommet to be screwed into the bone, whether there be a passageway there or via the use of self-tapping features. Alternatively, and/or in addition to this, a hex head can be utilized to enable a wrench to be utilized to apply the torque to the grommet. In an exemplary embodiment, a rotational driving force applied to the slot (or hex head) on the backside of the grommet causes the grommet to be screwed into bony tissue surrounding the cochleostomy and/or causing the grommet to be screwed into unopened bone in the case of the self-tapping grommet.

In an exemplary embodiment, there is a tool that enables the grommet or otherwise the body 810 to be supported at the end of the tool, and remain coupled to the tool in a manner that enables the tool to be utilized to transport the grommet to the location of insertion into the passage into the cochlea or for placement of the grommet against the bone so that the grommet can be utilized to self-tap a hole into the cochlea.

It is noted that any one or more of the features described above associated with the grommet can be present in the body 810, such as, for example the flats or the hex head. It is also noted that while the grommet detailed above has been described in terms of utilization with a cochlear implant electrode array, in some other embodiments, any one or more of the other second modules detailed herein can be utilized with the grommet.

While the embodiment detailed above is focused on an electrode array of a cochlear implant, in an alternate embodiment, the component that is inserted through the body 810 or other device that establishes the inner ear port device can be a direct acoustic stimulator. Alternatively, and/or in addition to this, electrodes that treat tinnitus and/or balance or some other device that treats tinnitus and/or balance, such as a mechanical actuating device, can be inserted through the port device. And this can include embodiments where the port device is utilized to provide access to the interior of the semicircular canals and/or the vestibule (and is thus mounted on and/or through the walls of such. Still, at least some exemplary embodiments are directed to providing a port device that enables access at the scala tympani side of the cochlea.

It is noted that in some exemplary embodiments, a plurality of the inner ear port device can be utilized. In an exemplary embodiment, there could be a port device that is dedicated for the use of the cochlear implant, and then another one that is utilized for therapeutic substance delivery, or sensing, etc. In an exemplary scenario, a first inner ear port device is implanted into a human, and it is utilized for monitoring the inner ear of the human. Upon a determination based on the monitoring that there can be utilitarian value with respect to providing a therapeutic substance, one or more of the therapeutic substances detailed herein are provided, such as, by way of example, by attaching a reservoir or a therapeutic substance containing component to the inner ear port device. This could entail removal of the sensor module of the device and replacing such with the reservoir module/therapeutic substance delivery module, which could include components to enable the continued sensing function of the now removed sensor module. Alternatively, this could entail opening a passageway to the inner ear and attaching the therapeutic substance delivery module in addition to the existing sensing components of the implant. The therapeutic substance can be delivered as utilitarian. Then, at some point in the future, the person's hearing degrades, despite the application of the therapeutic substance for example, and thus a cochlear implant is deemed to be utilitarian. The sensing module or the therapeutic substance delivery module or both are removed in some embodiments, and a cochlear implant electrode array is provided through the port device. Because there is still utilitarian value with the specimen to sensing and/or providing therapeutic substance, a second inner ear port device is added at a location away from the first inner ear port device, and thus the functionality of the first inner ear port device is achieved by the second inner ear port device.

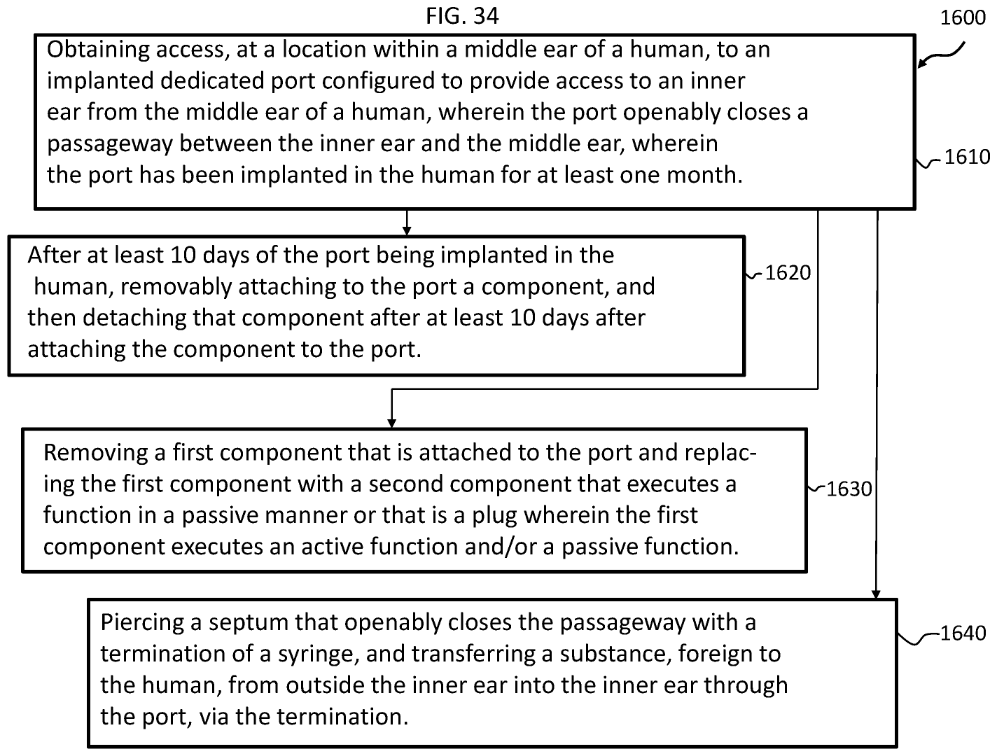

At least some embodiments includes methods. By way of example, FIG. 34 presents an exemplary algorithm for an exemplary method, method 1600 according to an exemplary embodiment. Method 1600 includes method action 1610, which includes the action of obtaining access, at a location within a middle ear of a human, to an implanted dedicated port configured to provide access to an inner ear from the middle ear of a human, wherein the port openably closes a passageway between the inner ear and the middle ear, wherein the port has been implanted in the human for at least one month, and in some embodiments, the port has been implanted in the human for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or weeks, or months, or years, or any value or range of values therebetween in one day/week/month/year increments. That is, the action of obtaining access is executed at least the noted days or weeks or months after the implanted dedicated port was fully implanted in the human.

The port can be any of the port devices detailed herein and/or variations thereof, providing that they enable the openably closable feature. This is as distinguished from, for example, the naked body 810 shown in FIG. 15 without the electrode array. That said, as shown in FIG. 35, an exemplary embodiment of this port according to method action 1610 is seen with a seal apparatus that includes a cap 1717. Thus, in an exemplary embodiment of executing method action 1610, the port device 1700 is the port that is the subject of the method action. In an exemplary embodiment of this method, the method includes removing the seal apparatus from the port device, thereby obtaining access to the passageway into the inner ear. Here, the cap 1717 is the mode of sealing. However, in an alternate embodiment, the body 1788 can be the mode of sealing (where body 1788 would correspond to a plug). In some embodiments, both a cap function and a plug function can be achieved via the seal apparatus.

In exemplary embodiment, the seal apparatus 3555 is screwed into the passageway (or can be interference fitted in other embodiments, as a cork), and thus there are mating threads within the passageway—the body 1788 is made of an elastomeric material which also establishes a seal between the threads and/or at the head—in an exemplary embodiment, the seal apparatus is a composite component where the head is made of an elastomeric material, and the threaded body is made of titanium. FIG. 35 shows an example of this seal apparatus 3555, where the head 1722 is pulled against the proximal surface of the body 810, thus establishing a seal, as a result of the threaded body being screwed into the passageway towards the distal end thereof (the threaded body is 1788—non-threaded cylinder 1744 links the head to the threaded body 1788). In an exemplary embodiment, such as that shown in FIG. 35, the tension established by the continued threading of the seal apparatus 3555 into the passageway causes the head 1722 to flex as shown, further reinforcing the sealing feature—the natural/relaxed state of the head 1722 is a rectangular shape. In a further exemplary embodiment, ribs or a protrusion can be located on the proximal facing surface of the body 810, which can “cut” into the elastomeric material of the head 1722. But to be clear, in some other embodiments, the threaded body 1788 can also be made of the elastomeric material, where the interference between the rigid threads within the passageway through body 810 and the elastomeric threads establish a seal. Thus, it can be seen that embodiments are directed to a component releasably attached to the tissue interface portion (e.g., body 810) and/or a portion of the overall implanted device supported by the tissue interface portion. In the embodiments herein, the component provides one or more passive features, such as providing a self-healing septum feature as detailed above. It is briefly noted that embodiments can include an implantable device that includes the tissue interface portion, the passive component and a sealing apparatus 3555. In this regard, FIG. 35 shows a seal apparatus 3555 and a passive component 3594 co-located with each other as part of the overall implanted device 1700. Embodiments can explicitly exclude a separate sealing apparatus 3555 (the passive component can also have a sealing function, concomitant with some of the embodiments detailed above).

In an exemplary embodiment, the device is configured, when the seal apparatus is removed from the device, so that the passageway is open when the component is releasably attached to the tissue interface portion and/or a portion of the device is supported by the tissue interface portion. In this regard, the component with the passive functionality does not seal the passageway. Conversely, in an exemplary embodiment, the component with the passive function is configured to unsealably seal a local portion of the passageway when the component is releasably attached to the tissue interface portion and/or a portion of the device is supported by the tissue interface portion. Thus, the sealing apparatus 3555 could be present, but the component with the passive function seals the local portion as well, thus providing redundancy. But even if the sealing apparatus 3555 was removed, the component with the passive functionality would be sealed. In an exemplary embodiment, the component is configured, when the seal apparatus is removed from the device, to unsealably seal the passageway when the component is releasably attached to the tissue interface portion and/or a portion of the device is supported by the tissue interface portion.

Returning back to the method 1600. In an exemplary embodiment of method 1600, the port that is implanted has been implanted in the human for at least a month, while in some embodiments, the port has been implanted for any one or more of the aforementioned time periods noted above.

In view of the above, it can be seen that method 1600 further includes the optional method action 1620, which includes, after at least X length of time of the port being implanted in the human, removably attaching to the port a component, and then detaching that component after at least Y length of time (or less than Y length of time in an alternate embodiment) after attaching the component to the port. In an exemplary embodiment, X and/or Y (the two need not be equal) can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or weeks, or months, or years, or any value or range of values therebetween in one day/week/month/year increments. In an exemplary embodiment, this component could be the above-noted electrode array. In an exemplary embodiment, this component could be a therapeutic drug delivery device that provides therapeutic substances. In an exemplary embodiment, this component can be any of the components detailed herein that are attached to the port that is directly attached to the tissue. As will be understood, embodiments of this embodiment can be directed towards swapping out a therapeutic substance delivery device for another, such as to provide a different therapeutic substance. Alternatively, and/or in addition to this, such enables the replacement of a failed component or the replacement of an obsolete or semi-obsolete component with a new component. All without disturbing the tissue interface.

In an exemplary embodiment, the component is an outlet of a therapeutics substance delivery apparatus, and the method further includes transporting a therapeutic substance into the inner ear through the port via the delivery apparatus's attachment to the port.

Method 1600 further includes the optional method action 1630, which includes removing a first component that is attached to the port and replacing the first component with a second component that executes a function in a passive manner or that is a plug/cap or some sealing device, wherein the first component executes at least one of an active function or a passive function. The passive component can be any of the components detailed herein that are attached to the port that is directly attached to the tissue. In an exemplary embodiment, this can be identical in design to the first component that is removed, and thus an identical replacement component. By way of example, this can be a therapeutic substance delivery device that has a full reservoir for example, the replacement of which addresses the fact that the reservoir the first component has been depleted or is near depletion. In an exemplary embodiment, the first component removed could be the above-noted electrode array, and the later component could be the plug/cap (because the array is no longer useful, for example). In an exemplary embodiment, it could be that an initial surgical operation is executed so as to provide a therapeutic substance delivery device in the recipient that provides therapeutic substance to the cochlea is an effort to preserve the cilia for example. Here, there is a decent likelihood that the recipient's hearing will eventually fail owing to degradation of the cilia, but there is also a decent likelihood that the delivery of the therapeutic substance may prevent such, at least in the short term. Accordingly, there is utilitarian value with respect to implanting the long-term/permanent port.

In any event, method 1600 further includes the optional method action 1640, which includes piercing a septum that openably closes the passageway with a termination of a syringe, and transferring a substance, foreign to the human, from outside the inner ear into the inner ear through the port, via the termination. In an exemplary embodiment, the method further comprises, at least Z hours or days or months after the action of piercing the septum, executing another action, after the septum has self-healed, of piercing the septum with a termination of a syringe and transferring a substance, foreign to the human, from outside the inner ear into the inner ear through the port, via the termination. In an embodiment, Z can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or any value or range of values therebetween in 1 increment. The termination/syringe can be the same or can be different than that which was the case when executing method action 1640.

In an embodiment, the method(s) include piercing the septum with the termination, transferring the substance, foreign to the human, from outside the inner ear into the inner ear through the port, via the needle, and removing the termination from the septum and the passageway, wherein, the septum is a self-healing septum, and the method further comprises enabling the septum to self-heal to close the passageway after the actions of piercing, transferring the substance, and removing the termination. In an exemplary embodiment, this occurs at least Z times over a period of Z years (the Zs need not be equal).

It is briefly noted that while the embodiments herein are typically directed to a port that accesses the ducts of the cochlea, in other embodiments, the port is utilized to access the interior of the semicircular canals. Accordingly, any disclosure herein relating to access to the ducts of the cochlea corresponds to a disclosure of accessing the interior of the semicircular canals for the purposes of textual economy unless otherwise noted, providing that the art enables such.

In an exemplary embodiment, there are method actions that include, and there are devices and/or systems that enable, repeated access to the inner ear, such as to the cochlea, or the vestibule ducts, more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, or 100 times or more, or any value or range of values therebetween in one increment in a time period spanning 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months, or years, or any value or range of values therebetween in one month increments.

Embodiments include devices and systems that enable, and methods of, accessing perilymph and/or other fluids, directly (as opposed to indirectly) of the inner ear repeatedly in a safe manner, along a path or route that corresponds to that which was previously the case to do so, in some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80, 90, or 100 times or more, or any value or range of values therebetween in one increments.

Embodiments can enable the concept of treatment of the inner ear where only one portion of the inner ear and/or the barrier that establishes a barrier between the inner ear and the middle ear, is put “at risk” at one time. Accordingly, if a problem arises, and the implant and/or a device cannot be utilized, a workaround can be implemented at another, “virgin” location.

In an exemplary embodiment, the teachings detailed herein are utilized as part of a method to, and/or the teachings detailed herein are utilized with a device and/or system configured to, treat Meniere's Disease and/or another chronic disease and/or to treat age-related hearing loss. In an exemplary embodiment, the teachings detailed herein are utilized as part of a method to, and/or the teachings herein are utilized with a device and/or system configured to, treat tinnitus, such as by way of example, suppress the perception of tinnitus. In an exemplary embodiment, the teachings detailed herein are utilized as part of a method to, and/or the teachings herein are utilized with a device and/or system configured to, treat an autoimmune scenario with respect to the inner ear, or some other inner ear disease, or a disease that affects otherwise has a deleterious effect on the function of the inner ear. By way of example only and not by way of limitation, embodiments can include enabling the provision of a steroid being supplied to the inner ear all the time.

By way of example only and not by way of limitation, exemplary method actions include providing a therapeutic substance at an efficacious level and/or at a level that can be measured to be an amount that is statistically efficacious, for at least 60, 65, 70, 75, 80, 85, 90, 91, 92, 93, 94, 95, 96, 97, 98, 99, or 100%, or any value or range of values therebetween in 1% increments of a collective number of hour or day increments over any one or more of the aforementioned temporal periods herein (e.g. the efficacious level exists in at least 21 hours out of each day in a 3 month period). And exemplary devices and/or systems include devices and/or systems that can enable such. In this regard, the aforementioned method can be executed utilizing one or more of the devices detailed herein and/or variations thereof.

By way of example only and not by way of limitation, in an exemplary embodiment, the teachings detailed herein are utilized as part of a method to treat balance and/or vertigo. In an exemplary embodiment, the teachings detailed herein are executed to enable a human who previously was not able to drive a vehicle or otherwise operate machinery in a safe manner, including in a scenario where a licensed organization or a supervisory organization (e.g., a Department of Motor Vehicles) previously deemed the person unable to do so. Accordingly, exemplary methods include receiving permission from such organizations to again continue executing one or more of these actions.

In an exemplary embodiment, the methods herein include attaching and unattaching various apparatuses to/from the port at least Z times over a period of Z years (the Zs need not be equal). And consistent with the artificial round window device detailed above, methods can include regulating pressure within the inner ear via an artificial round window assembly attached to the port. And consistent with the embodiment of FIG. 32, methods include regulating/adjusting the pressure in the middle ear to provide therapeutic substance to the inner rear.

Some embodiments include the action of utilizing the inner ear port device for insertion of a catheter for drug/therapeutic substance delivery in the cochlea, and providing such to the cochlea. In an exemplary embodiment, this action can be deep drug/therapeutic substance delivery into the cochlea

In an exemplary embodiment, there is the action of inserting a catheter into the passageway in the inner ear port device. In an exemplary embodiment, the catheter is inserted through the port device and into the cochlea at least 1, 2, 3, 4, 5, 6, 7, 8, 9, 10, 15, 20, 25, 30, 35, 40, 45, 50, 50 or 60 mm (where after about 30 mm, the catheter starts heading back towards the oval window) or any value or range of values therebetween in 1 mm increments, and then providing the therapeutic substance into the cochlea at any one or more of those locations. In an exemplary embodiment, the port device can be utilized for a visual scope or a sensing device that is temporarily inserted into the cochlea to perform a visual and/or other type of sensing inspection of the interior of the cochlea.

In an exemplary embodiment, the inner ear port device includes a magnetic alignment feature that can enable the action of blindly finding the inner ear port device utilizing a trans tympanic membrane approach. By way of example only and not by way of limitation, FIG. 35A presents an exemplary scenario where, for example, termination 2599 extends through the ear canal 102 and through the tympanic membrane 104, such that the entire tip and a portion of the shank of the termination is located in the inner ear cavity 106. The termination is part of an insertion tool that enables or otherwise includes “snake” 2567. Snake 2567 is a guidewire like device that is flexible and is hollow and configured to provide for transportation of the therapeutic substance from the tool or from a location remote from the tool, through the snake 2567, and out the exit of the stake 2567. At the end of snake 2567 is a nozzle that includes two “C” shaped magnets (the Cs are exactly half circles). The polarity of these magnets is opposite one another. As shown, the darker colored magnet is such that the north pole faces towards the rest of the snake and the light colored magnet is such that the north pole faces away from the rest of the snake.

In some embodiments, there is a drug elution portion of the port that keeps the port open at the inner ear side. For example, a passive component 3501 can include a ring 3551 of long lasting slow release steroid, NSAID or antifibrotic that prevents fibrosis blocking the port is located as seen in FIG. 35. In this exemplary embodiment, a support structure 3594 is located outboard of the ring 3551, and is interference fitted or welded to the port. This has utilitarian value in that it can prevent the dissolution of the ring 3551 at the outboard portions thereof and otherwise can retain the ring within the port for a longer period of time otherwise than that which would be the case if the perilymph or the like could access the sides and/or the bottom of the ring. Still in some embodiments, the ring 3551 can be located in the port without the support structure 3594, albeit with respect to a larger outer diameter of the ring. In some embodiments, the ring could be located on the outside of the port structure. This can be seen in FIG. 35 as well, with respect to ring 3552. Here, ring 3552 is located outboard of the port at the end of the port, and sized and dimensioned so that the port can be extended through the opening in the barrier between the middle here in the inner ear without disturbing the ring 3552. This can have utilitarian value with respect to scenarios where the fibrous tissue growth encroaches from the barrier between the middle ear and the inner ear outward towards the distal end of the port. When the fibrous tissue reaches the ring 3552, further encroachment could be halted or otherwise slowed. Indeed, in an exemplary embodiment, the ring can be made of a material or otherwise can be configured so that the therapeutic substance delivered by the ring is not delivered until the therapeutic substance comes into contact with the ring.

Inner ear port 2500 is shown located in the lower portion of the cochlea below and to the right of the round window 121. This feature is different than the arrangement shown in FIG. 7, where the port 700 accesses the side of the cochlea where the oval window is located. As seen, inner ear port device 2500 includes a magnet arrangement that corresponds to that of the snake 2567. Accordingly, when the snake 2567, or more specifically, the end of the snake 2567 reaches a location proximate the port 2500, the magnetic attraction between the two components will cause the snake to the moved towards an otherwise guided on to the port 2500 in an aligned manner, owing to the polarity of the magnets in the arrangements of the magnets on each of the components. Upon sufficient coupling, the therapeutic substance can be transferred from the snake to the port 2500, and into a reservoir, for example, to refill the reservoir. Accordingly, by utilizing the magnets, in an exemplary embodiment, the snake 2567 can be “blindly” inserted into the middle ear, and potentially at least towards a rough idea of where the port is, and then the magnetic attractions takeover to guide the snake to the port. Accordingly, exemplary embodiments can include accessing the port and transferring a therapeutic substance to the port device without being able to visually see the port, whether directly or by utilizing a camera for that matter.

In an exemplary embodiment, there can be a magnetically actuated valve that is located in the port device 2500, that opens when the snake 2567, or more specifically, when the magnets of the snake 2567 become located proximate the port, thus to enable the transfer of the therapeutic substance from the snake to a reservoir. The magnetically actuated valve can be such that when the snake is pulled away from the port 2500, such as by way of example, when the snake 2567 is pulled back through the termination 2599, the absence of the magnetic field will cause the valve to close. It is noted that the magnetic field could cause this opening, and thus can provide the “force” to open and close the valve, while in other embodiments, the port device 2500 can include a sensor or otherwise an electronic logic circuit that, when the magnetic field is sensed, the port device 2500 controls itself to open. That said, instead of utilizing magnetic fields per se, a signal could be provided from the snake to the implant 2500 instructing the implant 2500 to open the valve. Such an arrangement can be utilized with embodiments that do not necessarily utilize the snake 2567. For example, if a termination is utilized to directly access the port device, a communication signal can be provided from external the recipient and/or from internal the recipient (an antenna can be located on the termination), to the port device, to instruct the port device to open the valve and/or close the valve.

Corollary to this is that in some exemplary embodiments, the valve of the port can be spring loaded or otherwise biased close, and then when a male portion of the snake and/or the termination enters the passage, the male portion pushes the valve open, and then upon withdrawal, the valve “springs” shut, thus preventing the therapeutic substance for example, from entering the middle ear or otherwise escaping from the cochlea, or otherwise limiting the amount of perilymph escape relative to that which would otherwise be the case.

And in a further embodiment, there can be a mechanical actuation system that opens the valve and closes the valve. By way of example only and not by way of limitation, there could be a receptacle for a screw driver or a hex driver or the like on the portion of the port that is located in the middle ear, and by turning this receptacle, because a mechanical linkage between the receptacle and the valve, the valve can be turned open and turned closed.

Any device or system that enables and/or any method of guiding a therapeutic substance delivery device to the port device and/or any device or system that enables and/or method of opening and/or closing a valve so the reservoir can be refilled can be utilized in at least some exemplary embodiments providing such is enabled by the art unless otherwise noted.

And further, while the above embodiment has been described in terms of transferring therapeutic substance that is in the form of a fluid, in other embodiments, the therapeutic substance can be in a solid form or otherwise contained in a solid container. In this regard, solid pellets or solid containers can be pushed through the snake, by a guidewire for example, and then into the port device.

In view of the above, it can be seen that in some exemplary embodiments, such as where the grommet is utilized to establish the tissue interface component, or body 810 in an alternative embodiment, or any other arrangement where a body or grommet is firmly screwed into the bony tissue through which the cochleostomy is made (where the action of screwing could establish the cochleostomy in the case of a self-tapping grommet or self-tapping body 810), which body or grommet establishes a passageway from the middle ear to the inner ear, this passageway can be utilized as an access hole for one or more purposes, such as by way of example only and not by way of limitation, the utilization of the delivery of the desired or needed drugs steroids fluids and/or tissue growth inhibiting substances, all by way of example, to the inside of the cochlea. And again, in at least some exemplary embodiments, there are methods where, when access is not needed, the access hole is plugged or otherwise sealed to prevent fluid within the cochlea from escaping into the middle ear.

In an exemplary embodiment, the implanted dedicated port is part of an implant that is configured to release a transferred substance into the inner ear over a period of at least 0.25, 0.5, 0.75, 1, 1.25, 1.5, 1.75, 2, 2.5, 3, 3.5, 4, 5, 6, 7, 8, 9, 10, 11, 12, 13, 14, 15, 16, 17, 18, 19, 20, 21, 22, 23, or 24 hours or 1, 2, 3, 4, 5, 6, 7, 8, 9, 10 days or 1, 2, 3, 4, 5, 6, 7, 8, 9, 10, 11, 12, 13, 14, 15 or more weeks or months, or any value or range of values therebetween in 0.1 hour increments, using the port.

In an exemplary embodiment, the action of obtaining access is executed at least 1, 2, 3, 4, 5, 6, 7, 8, 9, 10, 11, 12, 13, 14, 15, 20, 25, 30, 35, 40, 45, 50, 60, 70, 80, 90 or 100 days or weeks or months or any value or range of values therebetween in one day increments after the implanted dedicated port was fully implanted in the human.

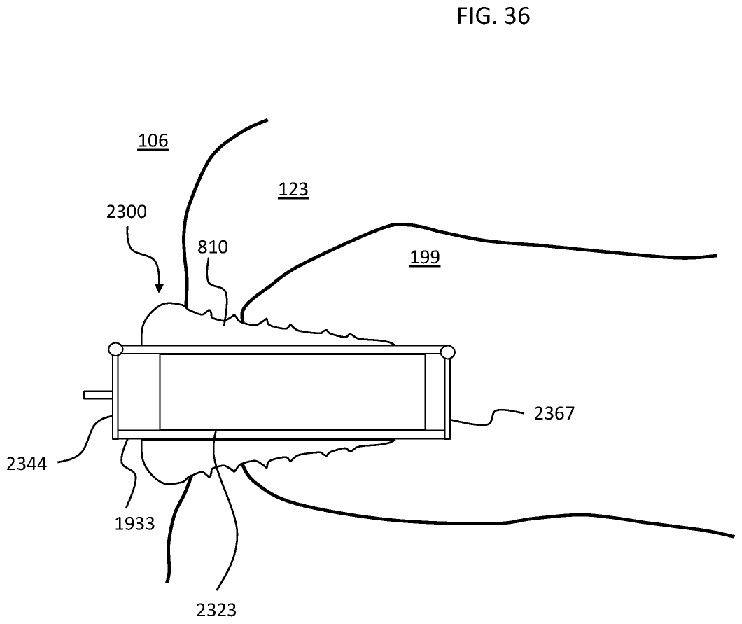

FIG. 36 presents another exemplary embodiment of an inner ear device 2300. Here, there is the tissue interface body 810, which can correspond to the body detailed above. Here, the second module is established by a tube 1934, and there is a hinged spring loaded door 2344 with a nub facing outward to enable a tweezers or the like to grip such and provide a force to counteract the spring load to open the door. This is an exemplary embodiment where the artificial round window or oval window is located at the proximal end of the device. This exemplary embodiment of implant 2300 further includes a therapeutic substance delivery submodule 2323 of the second module. In this exemplary embodiment, element 2323 can be a reservoir that contains a therapeutic substance, where the therapeutic substance is configured to defuse through the outer wall thereof. In an exemplary embodiment, element 2323 can be a body of the therapeutic substance in a solid configuration, and the solid configuration will dissolve or elute when exposed to the perilymph. This is controlled by controllably opening and closing door 2367, where door 2367 is biased closed, but pressure changes within the tube 1933 will force the door open periodically to implement therapeutic substance delivery. In a self-perpetuating manner, opening of the door for a period of time to allow perilymph to flow into the second module causes the perilymph to react with an agent in module 2323, which again increases the pressure, and so on.

Still further, embodiments can include the delivery of solid bodies that contain a therapeutic substance in a fluid state, where the solid bodies can dissolve to thus release the deputy substance therein. Alternatively, and/or in addition to this, the solid bodies can split otherwise open to release the therapeutic substance when in the interior of the cochlea.

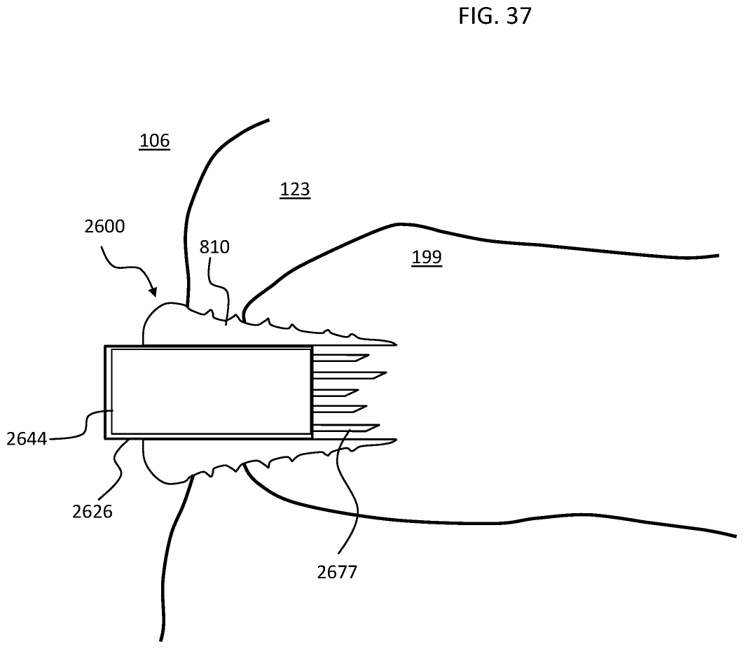

FIG. 37 provides another exemplary embodiment of an inner ear device 2600, that includes the body 810 detailed above, into which is removably threaded a second module, which includes a housing 2626, in which is located a reservoir 2644 containing a therapeutic substance. The reservoir 2644 is in fluid communication with a plurality of delivery ports 2677. In an exemplary embodiment, a porous membrane can be located between the therapeutic substance contained in the reservoir and the delivery ports, to meter or otherwise slow the delivery of the therapeutic substance. In an exemplary embodiment, the reservoir can be pressurized to deliver therapeutic substance from the one or more delivery ports 2677. In an exemplary embodiment, the housing 2626 can be unscrewed from the body 810, and then a new housing can be replaced with additional therapeutic substance or new types of therapeutic substance. Further, in an exemplary embodiment, the housing can be opened and the reservoir 2626 could be swapped out with a new reservoir. In an exemplary embodiment, the aforementioned membrane can be such that the membrane permits transfer of fluid in only one direction (i.e., into the cochlea), so that when the reservoir is removed for replacement, but perilymph does not leak or otherwise escape out of the cochlea, or otherwise limits the amount of perilymph that could leak out of the cochlea relative to that which would otherwise be the case.

Embodiments can also include a method of accessing the cochlea utilizing a traditional cochleostomy and/or by entering the cochlea utilizing the round window or utilizing the oval window, which cochleostomy or entrance by the round window or entrance by the oval window is utilized to insert a cochlear implant electrode array into the cochlea. Then, during the same procedure of accessing the cochlea as just noted, a second cochleostomy or a first cochleostomy is established for the inner ear port device. This could enable future access to the cochlea without having to perform another entire surgery to access the cochlea. That is, by way of example only and not by way of limitation, this can be analogous to changing a timing belt when accessing other portions of an engine for maintenance or repair, even though the timing belt does not need to be changed per se. That is, the difficulty in accessing the location far outweighs the de minimis nature of taking an action that never has any future utility in practice and and/or utilizing a device that will never be used in the future or ever at all. Accordingly, exemplary embodiments include inserting a cochlear implant electrode array through a first passage, and inserting an inner ear port device into a second passage from the middle ear into the inner ear, and not utilizing that inner ear port device for one or more or all of its intended purposes for at least 1, 2, 3, 4, 5, 6, 7, 8, 9, or 10 years or more, or any value or range of values therebetween in one month increments from the time of implantation. In an exemplary embodiment, the inner ear port device is utilized at the time of implantation, but that is not utilized again for any one or more of the aforementioned temporal ranges.

In an exemplary embodiment, there is the aforementioned tissue interface body that provides a passage from the middle ear to the inner ear, where the passage is plugged/sealed for any one or more of the aforementioned temporal periods. That is, in an exemplary embodiment, the access hole is plugged when not in use. In an exemplary embodiment, the access hole is plugged in a manner that prevents any impact on the auditory system.

In an exemplary embodiment, the port device is completely unrelated to any function in the auditory system.

An exemplary method includes utilizing a body that comprises a biosuitable material to establish a permanent tissue interfacing implant that provides a passageway from the middle ear into the inner ear. In an exemplary embodiment, the aforementioned biosuitable material causes a mammalian inflammatory response, and this can be utilitarian with respect to providing a seal between the tissue (wall of the cochlea through which the body passes) and the body. In any event, at least some exemplary embodiments of the tissue interfacing body integrate in a utilitarian manner with the cochlear bony wall. A second component or second module is placed or otherwise is located in the passageway so as to fluidically seal the cochlea with respect to the passage that has been created, in which the implant is located.

It is noted that at least some exemplary embodiments include providing a “universal” tissue interface body that establishes a passage between the middle ear and inner ear. By way of example only and not by way of limitation, this can correspond to the body 810 detailed above by itself. The body can include a threaded passage therethrough, into which the threaded passage can initially be a threaded plug or cap that will seal the passage and prevent fluid leakage from the inner ear to the middle ear. This plug or cap can be considered a second module, and can be replaced with, in the future, another module that has one or more of the features and/or structural components detailed herein, or any other functional or structural component that can have utilitarian value. This can enable the functionalities to be changed in accordance with temporally changing needs of a recipient. Alternatively, and/or in addition to this, embodiments include a kit arrangement where, for example, the kit includes a tissue interface component, such as body 810, and then a number of different second modules that have various functionalities. In an exemplary embodiment, this can enable a surgeon or otherwise a healthcare professional to basically “build” an implant according to the needs at the time of assessment. By way of example, a kit could permit the establishment of a therapeutic substance delivery device or a sensor device etc., where the surgeon could “screw” a second component (or a first component, for that matter) into the threaded passage of the body 810, that corresponds to one or more or all of the components that are attached to the body 810 detailed herein. This can be done before or after implantation of the body 810. By way of example only and not by way of limitation, the surgeon could “build” a combined therapeutic substance delivery system and a cochlear implant system. Any one or more of the combinations detailed herein can be combined with any one or other of the combinations detailed herein providing that the art enables such unless otherwise indicated. Accordingly, embodiments include methods of establishing any one or more of the combinations any one or more of the features detailed herein, and these methods can be executed by a healthcare professional such as the surgeon or someone under the supervision of the surgeon or otherwise working with the surgeon, and this can be done within 1, 2, 3, 4, 5, 6, 7, 8, 9, 10, 11, 12 hours or 1, 2, 3, 4, 5, 6, 7, 8, 9, 10 days of the implantation of the device, and can be done within half a mile or within a mile of the implantation site.

It is also noted that some exemplary embodiments include the ability to “lock” the second module, whatever it is, to the first module. In an exemplary embodiment, upon locking, the second module cannot be removed from the first module without removing the first module from the tissue/bone.

Thus, in an exemplary scenario of use, the port devices are used for the sole purpose of delivering drugs to the inner ear of patient/recipient for a first period of time and then the port device facilitates the insertion of a cochlear implant electrode array at a later time point in time. The use of the cochlear implant electrode array can be utilized simultaneously with further therapeutic substance delivery and/or sensors associated with the port device. Thus, in an exemplary embodiment, there is a port device that has dual (at least dual) or triple (at least triple) functionality. In an exemplary embodiment, the utilizations of the functionalities are staggered, and in some embodiments, the utilizations can overlap. In an exemplary embodiment, any one or two of the functionalities are utilized for at least 1, 2, 3, 4, 5, 6, 7, 8, 9, 10, 11, 12 days, or weeks, or months, or years, or any value or range of values therebetween in one increments of the associated unit, and then anyone all other or two other of the functionalities are then implemented and utilized (the initial one or two functionalities are not utilized prior, aside from, potentially, testing and validation that such will be utilitarian when later implemented).

It is noted that any reference herein to a therapeutic substance corresponds to a disclosure of an active substance such as an active drug or an active biologic etc., and any disclosure herein to an active substance such as an active drug or the phrase active substance in the generic manner corresponds to a disclosure of an active biologic or a therapeutic substance, etc. Any active pharmaceutical ingredient that can have utilitarian value can be a therapeutic substance. Proteins can be therapeutic substances as well. It is also noted that in an at least some exemplary embodiments, an inactive fluid can be a physiological saline, which can be utilized to convey the therapeutic substance into the cochlea.

In an exemplary embodiment, therapeutic substance include but are not limited to, any of those detailed above, and can include peptides, biologics, cells, drugs, neurotrophics, etc. Any substance that can have therapeutic features if introduced to the cochlea can be utilized in some embodiments.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Embodiments include embodiments where any or more of the teachings detailed herein can be combined with any one or more of the other teachings detailed herein unless otherwise noted providing that the art enables such. Embodiments also include embodiments where any one or more of the teachings detailed herein is specifically excluded from combination with any one or more of the other teachings detailed herein almost otherwise noted providing that the art enables such

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. It is also noted that embodiments can include devices systems and/or methods that specifically exclude one or more of the disclosures presented herein (i.e., it is not present).

While various embodiments of the present invention have been described above, it should be understood that they have been presented by way of example only, and not limitation. It will be apparent to persons skilled in the relevant art that various changes in form and detail can be made therein without departing from the scope of the invention.

1. A device, comprising:
a tissue interface portion configured for securement to tissue of and/or proximate an inner ear of a human and provide a passage from outside the inner ear to inside the inner ear; and
a component releasably attached to the tissue interface portion and/or a portion of the device supported by the tissue interface portion, wherein
the device is configured to enable the component to be removed from the tissue interface portion when the tissue interface portion is removably permanently fixed to the barrier establishing the inner ear of a human, and
the component at least partially seals the passage and provides one or more passive features.

2. The device of claim 1, wherein:
the component is a therapeutic substance delivery device.

3. The device of claim 1, wherein:
the component is an artificial round window.

4-5. (canceled)

6. The device of claim 1, further comprising:
a guide configured to guide a tip of a termination of a syringe located outside the inner ear to the septum and/or a passage of the tissue interface portion leading to the septum.

7. (canceled)

8. The device of claim 1, wherein:
the component is a therapeutic substance delivery apparatus;
the device is configured so that a water-based substance natural to the inner ear can enter the delivery apparatus;
the delivery apparatus is configured so that an internal pressure of the delivery apparatus is increased due to the entry of the water-based substance, which pressure increase results in delivery from the delivery apparatus of the therapeutic substance into the inner ear.

9. The device of claim 1, wherein:
the tissue interface portion has been implanted into bone establishing a barrier between the middle ear and the inner ear for at least 2 years; and
the component was implanted for at least eighteen months less than the tissue interfacing portion.

10. The device of claim 1, wherein:
the component completely seals the passage.

11. A system, comprising:
a tissue interface apparatus through which a passage extends, wherein the tissue interface apparatus is configured to permanently fix to an opening in a barrier between a middle ear and an inner ear of a human such that the passage extends from the middle ear to the inner ear; and
a first functional component at least partially located in the passage, wherein
at least one of:
the first functional component is a passive first functional component detachably attached directly or indirectly to the tissue interface apparatus and removal of the first component provides physical access from the middle ear into the inner ear through the passage, wherein the first functional component at least partially seals the passage in addition to providing a passive feature;
the tissue interface apparatus includes a second passage that is unsealably sealed, the second passage providing physical access from the middle ear into the inner ear through the second passage bypassing the first functional component; or
the first functional component has a passage that is unsealably sealed, the passage of the first functional component providing physical access from a middle ear facing side to an inner ear facing side when unsealed with the first functional component in the passage of the tissue interface apparatus.

12. The system of claim 11, wherein:
the first functional component is the passive first functional component detachably attached directly or indirectly to the tissue interface apparatus and removal of the first component provides physical access from the middle ear into the inner ear through the passage, wherein the first functional component at least partially seals the passage in addition to providing a passive feature;
the first function component is an artificial round window; and
the inner ear barrier tissue interface apparatus is located at a natural round window location of a human and the artificial round window replaces a natural round window of the person.

13. The system of claim 11, wherein:
the first functional component is the passive first functional component detachably attached directly or indirectly to the tissue interface apparatus and removal of the first component provides physical access from the middle ear into the inner ear through the passage, wherein the first functional component at least partially seals the passage in addition to providing a passive feature; and
the first functional component is a passive drug-release device.

14. The system of claim 11, wherein:
the first functional component is the passive first functional component detachably attached directly or indirectly to the tissue interface apparatus and removal of the first component provides physical access from the middle ear into the inner ear through the passage, wherein the first functional component at least partially seals the passage in addition to providing a passive feature; and
the first functional component is a self-healing septum.

15. The system of claim 11, wherein:
the first functional component is the passive first functional component detachably attached directly or indirectly to the tissue interface apparatus and removal of the first component provides physical access from the middle ear into the inner ear through the passage, wherein the first functional component at least partially seals the passage in addition to providing a passive feature; and
the first functional component is a therapeutic substance containing reservoir that is compressible due to atmospheric pressure increase in the middle ear, wherein compression of the first component due to the atmospheric pressure increase in the middle ear drives a therapeutic substance contained in the reservoir into the inner ear.

16. The system of claim 11, wherein:
the first functional component is the passive first functional component detachably attached directly or indirectly to the tissue interface apparatus and removal of the first component provides physical access from the middle ear into the inner ear through the passage, wherein the first functional component at least partially seals the passage in addition to providing a passive feature; and
the first functional component is a therapeutic substance containing reservoir that has a self-healing septum facing the middle ear when attached to the inner ear barrier tissue interface and is in fluid communication with the passage.

17-19. (canceled)

20. The system of claim 11, wherein:
the first functional component has the passage that is unsealably sealed, the passage of the first component providing physical access from the middle ear facing side to the inner ear facing side when unsealed with the first functional component in the passage of the inner ear barrier tissue interface apparatus.

21-30. (canceled)

31. A device, comprising:
a tissue interface portion configured for securement to tissue of and/or proximate an inner ear of a human and provide a passage from outside the inner ear to inside the inner ear;
a removable seal apparatus configured to unsealably seal the passage; and
a component releasably attached to the tissue interface portion and/or a portion of the device supported by the tissue interface portion, the component providing one or more passive features, wherein
the device is configured to enable the component to be removed from the tissue interface portion when the tissue interface portion is removably permanently fixed to the barrier establishing the inner ear of a human.

32. The device of claim 31, wherein:
the component is a therapeutic substance delivery device.

33. (canceled)

34. The device of claim 31, wherein:
the component is a self-healing septum.

35. (canceled)

36. The device of claim 31, further comprising:
a termination over-insertion prevention device configured to prevent over insertion of a termination of a syringe extended through the septum into the inner ear.

37. The device of claim 31, wherein:
the component is a therapeutic substance delivery apparatus;
the device is configured so that a water-based substance natural to the inner ear can enter the delivery apparatus;
the delivery apparatus is configured so that an internal pressure of the delivery apparatus is increased due to the entry of the water-based substance, which pressure increase results in delivery from the delivery apparatus of the therapeutic substance into the inner ear.

38. The device of claim 31, wherein:
the tissue interface portion has been implanted into bone establishing a barrier between the middle ear and the inner ear for at least 2 years; and
the component was implanted for at least eighteen months less than the tissue interfacing portion.

39-60. (canceled)
